(19) United States
(12) Patent Application Publication
(10) Pub. No.: US 2009/0148503 A1
(43) Pub. Date: Jun. 11, 2009

(54) Topical devices and methods for assisting performance of surgical incision

(76) Inventor: Hai H. Trieu, Cordova, TN (US)

Correspondence Address: Hai H. Trieu, 1323 Graystone Lane, Cordova, TN, 38016, US

(21) Appl. No.: 12/284,383

(22) Filed: Sep. 22, 2008

Related U.S. Application Data
(60) Provisional application No. 60/998,587, filed on Oct. 12, 2007.
(60) Provisional application No. 61/133,744, filed on Jul. 2, 2008.

(51) Int. Cl. A61K 9/70; A61F 13/02

(52) U.S. Cl. 424/447; 602/54

(57) ABSTRACT

A flexible topical device for assisting performance of surgical incision comprises a tear-resistant support layer and an adhesive layer disposed thereon. The device can further comprises a tear-resistant reinforcement layer disposed adjacent to the support layer. The device is adhesively attached to a surface of the tissue in which an incision is to be made, adjacent to the incision. The device prevents or limits an initiation or propagation of a tear in the tissue resulting from the stresses or strains imposed on the incision.

### CROSS-REFERENCE TO RELATED APPLICATIONS

This present application claims the priority and benefit of U.S. Provisional Patent Application Nos. 60/998,587 filed on Oct. 12, 2007 and 61/133,744 filed on Jul. 2, 2008, which are incorporated by reference herein in their entirety.

### BACKGROUND OF THE INVENTION

The present invention relates to topical devices and methods for assisting the performance of a surgical incision. In particular, the present invention relates to topical devices and methods for reducing the risk of uncontrolled tear of a perineum following an incision.

The most common injuries to the vagina and the perineum during labor or natural childbirth delivery occur at the vaginal opening, which may tear due to excessive stretching as the baby's head passes through. The vaginal opening typically dilates to at least four inches for delivery. When dilation occurs slowly and gradually, the tissues are flexible or pliable enough to allow necessary stretching. However, when the baby descends too quickly, vaginal tissue tearing or laceration may happen. Lacerations of the vaginal opening most often occur near the midline in the posterior aspect or the part of the vagina closest to the anus, which will be referred to as perineal lacerations or lacerations of the perineum. Tears in the anterior area of the vaginal opening may also occur, but these lacerations are typically small.

Perineal lacerations are categorized from first to fourth degree in order of increasing severity. First degree lacerations, which are the most common, involve only the lining or mucosa of the vagina. Second degree lacerations involve the vaginal lining as well as deeper or submucosal tissues of the vagina. More serious lacerations involve the deeper tissues including the anal sphincter and the rectum. Third degree lacerations extend from the vaginal lining through the anal sphincter, but do not involve the rectal lining. Fourth degree tears include the vaginal lining, submucosal tissues, anal sphincter, and rectal lining.

The third and fourth degree lacerations are more challenging to repair and typically require a skilled surgeon. They are more likely to be associated with complications, including bleeding, infection, increased pain, and anal incontinence. When lacerations involve the anal sphincter, the resulted dysfunction of this muscle can cause leakage of stool or gas from the anus. Injury to the sphincter may result in long-term problems, such as fecal incontinence or the development of a recto-vaginal fistula (a small channel that connects the rectum with the vagina).

Increased risk of perineal lacerations is known to associate with women at their first vaginal delivery, delivery of a large baby, abnormal position of the baby's head, and forceps or vacuum-assisted vaginal delivery. Forceps-assisted deliveries are more likely to be complicated by vaginal wall tears and third or fourth degree vaginal lacerations.

One of the most significant risk factors that can lead to more severe lacerations is episiotomy, especially a midline or medial episiotomy. Episiotomy is an intentional incision of the vaginal opening to hasten delivery or to avoid or decrease potential tearing. Episiotomy is the most common procedure performed in obstetrics, with an estimate of one done in every three to four natural births. An episiotomy may be very beneficial in certain circumstances and may prevent the need for cesarean section or assisted vaginal delivery using forceps or a vacuum extractor. A midline or medial episiotomy is an incision of the vaginal opening along the midline, straight down toward the anus. Advantages of a midline episiotomy include lower blood loss, less pain, easy repair, and improved healing. A midline episiotomy is less likely to result in long-term tenderness or problems with pain during intercourse. However, the main disadvantage of a midline episiotomy is the likelihood for this type of incision to extend beyond the initial incision and result in a third or fourth degree tear, which involves injury or damage of the anal sphincter or the lining of the rectum.

It is believed that perineal injuries or tears may be reduced if delivery occurs in a controlled fashion, with the baby's head descending slowly and steadily through the vaginal opening. Unless there is evidence of fetal distress, letting the baby's head to rest at the vaginal opening for several minutes to allow adequate tissue stretching is thought to be beneficial. Perineum massage and warm compresses of vaginal tissues are techniques that have been tried to help the stretching process with limited success. Perineal lacerations may still occur even when good care is taken to avoid them. Although attempts have constantly been made over the years to prevent perineal trauma, there is presently no reliable method or device for preventing laceration during childbirth labor.

The pushing process by the mother during childbirth labor also results in significant forces or pressure in other tissues within or adjacent to the perineum, such as the anal sphincter muscles, the anus, the perianal tissues. As a result, the tissues can undergo excessive straining that may lead to tissue damages or trauma including strained perineal tissues, strained anal sphincter, anal tears, anal fissures, external hemorrhoid, internal hemorrhoid, prolapsed internal hemorrhoid, etc. Applied forces or pressure in the opposite direction along the sagittal plane in order to counter the pushing forces or pressure may require someone to manually push against the tissues or prevent the tissues from pushing forward along the sagittal plane with one or both hands. A device applying or resisting a compressive force or pressure along the sagittal plane against the perineum may be used to achieve the same objective, but it is not expected to help resist perineal laceration initiated at the vagina. Excessive compressive force or pressure by a device may adversely affect the tear resistance of the perineum. Furthermore, compression against the tissues in the perineum close to the vagina may adversely affect the delivery or the baby.

Limiting or preventing tissue damages including perineal laceration, especially the third or fourth degree laceration, is necessary to reduce trauma and complications associated with vaginal childbirth delivery. Previous attempts or techniques have yet proven to be effective or consistent in prevention or reduction of perineal laceration or other tissue damages. A need therefore exists for a device and a method to help reduce, limit or prevent laceration or tissue damages during natural childbirth delivery. The present invention addresses these needs.

### SUMMARY OF THE INVENTION

In general, the present invention provides topical devices for assisting the performance of a surgical incision and methods of making and using the same. A topical device of the present invention comprises a flexible patch that comprises a tear-resistant support layer and an adhesive layer disposed thereon.

In one aspect, a topical device of the present invention further comprises a high tear-resistant reinforcement layer disposed on the support layer or between the adhesive layer and the support layer.

In another aspect, a topical device of the present invention is adhesively attached, affixed, bonded, or secured to a tissue, adjacent to an incision that was or will be made in the tissue, wherein at least a portion of the topical device is disposed substantially nonparallel to the incision.

In still another aspect of the invention, topical devices and related methods are provided, wherein a topical device comprises a topical patch, which comprises a support layer and an adhesive layer. The topical device is configured to be adhesively attached, affixed, bonded, or secured to, at least, a portion of the perineum without covering the anal aperture in order to reduce, limit, or prevent perineal laceration. At least a portion of the support layer is made of a tear-resistant material to provide tear-resistant support to the perineal tissues. The topical device has a shape and size that are suitable for attaching, affixing, bonding, or securing to the perineal area between the vagina and the anus without covering the anal opening. The adhesive layer provides secured attachment of the topical patch to the skin or dermal tissues of the patients, and thereby can transfer the tensile and shear stresses from the underlying dermal tissues to the support layer. The topical patch is fixedly attached to the perineum substantially parallel to a plane that is substantially tangent or parallel to the perineum and substantially transverse to the sagittal plane. In some embodiments, a release liner covering the adhesive layer and being releasable therefrom is used to protect the adhesive layer prior to use. The release liner is typically removed and discarded before the topical patch is attached to the perineum.

In still another aspect, the topical device is flexible such that, when attached to the tissue, it can be formed to follow the contour of the tissue surface.

In a further aspect, the adhesive layer comprises a biocompatible adhesive. As used herein, the term “biocompatible” means incapable of inducing an adverse reaction in a patient on whom the device is attached. Such adverse reaction can include, for example, allergic reaction, inflammatory reaction, irritation, burning sensation, or itching.

In yet another aspect of the invention, the topical device is configured to be adhesively attached, affixed, bonded, or secured to, at least, a portion of the perineum and completely or substantially surround the anus without covering the anal aperture in order to reduce, limit or prevent perineal laceration and anal tissue straining, respectively. At least a portion of the support layer comprises a tear-resistant material in order to provide tear-resistant support to the perineal tissues. At least a portion of the support layer can optionally comprise a semi-stretchable or non-stretchable material to provide stretching-resistant support to the perineal tissues. The topical device has a shape and size that are suitable for attaching, affixing, bonding, or securing to the perineal area and the perianal area between the vagina and the anus without covering the anal opening.

In yet another aspect of the invention, a topical device is configured to be adhesively attached, affixed, bonded, or secured to, at least, a portion of the perineum, completely or substantially surround the anus and cover the anal aperture in order to reduce, limit or prevent perineal laceration, anal tissue straining, and anal tissue extrusion, respectively. At least a portion of the support layer comprises a tear-resistant material in order to provide tear-resistant support to the perineal tissues. At least a portion of the support layer can optionally comprise a semi-stretchable or non-stretchable material to provide stretching-resistant support to the perineal tissues. The support layer has a shape and size that are suitable for attaching, affixing, bonding, or securing to the perineal area and the perianal area between the vagina and the anus with substantial covering the anal opening. The adhesive layer attaches, affixes, bonds, or secures the topical device to the skin or dermal tissues of the patients, and thereby can transfer the tensile and shear stresses from the underlying dermal tissues to the support layer. In certain embodiments, a release liner can be included and used, as disclosed hereinabove.

In still other aspects of the invention, topical patches of selected configurations (e.g. shape, size, thickness, materials, reinforcement, etc.) when used according to a method of the present invention can reduce, limit or prevent midline or medial laceration. Topical patches of appropriate configurations when secured at an appropriate location on the perineum according to a method of the present invention can reduce, limit or prevent medial episiotomy cut from extending further toward the anus. Topical patches of selected configurations when used according to a method of the present invention can reduce, limit or prevent third or fourth degree laceration. Topical patches of appropriate configurations when used according to a method of the present invention may also divert or deflect laceration from midline or medial direction to mediolateral direction. Topical patches of appropriate configurations when used according to a method of the present invention can bias a diverted mediolateral laceration toward a predetermined side or direction. Topical patches of appropriate configurations when used according to a method of the present invention can divert laceration to mediolateral direction and then limit such laceration from further propagation or advancement after extending to a certain length.

In still another aspect of the invention, methods of use of the topical devices are provided. A method includes adhesively attaching or affixing a topical patch of the present invention onto the perineum before or during natural childbirth delivery, wherein an adhesive layer of said topical patch comes into contact with said perineum. The method of use may further include preparing the perineum area prior to attaching or affixing the topical patch, wherein preparing the perineum area includes shaving, waxing, washing, cleaning, disinfecting, conditioning, treating, anesthetizing, wiping, drying, or a combination thereof. The method may also include removing the release liner in multiple stages. The method may further include orienting and positioning the topical patch prior to patch placement. The method may include using additional adhesive or adhesive tape to further secure or reinforce the topical patches. The method may include removing the patch after delivery.

The method concerning the use of topical devices or topical patches may optionally include additional support by an applicator to: 1) facilitate placement and bonding of the topical patch onto the perineum prior to or during childbirth delivery; 2) improve or maintain adhesive bonding of the topical patch to the perineum during delivery; 3) prevent premature debonding or delamination of the topical patch during delivery; 4) fix, repair or reapply premature debonding or delamination of the topical patch during delivery; or 5) remove the topical patch after delivery. The method may also include direct support of the topical patch by one or both hands of a medical staff instead of using the applicator. Other features and advantages of the present invention will become apparent from the following detailed description, claims, and the appended drawings.

### BRIEF DESCRIPTION OF THE DRAWINGS

FIG. 1 shows the midline or medial cross-section of the perineum along the sagittal plane.

FIG. 2 shows the front view of the perineum that is substantially transverse to the sagittal plane as the patient is lying down on her back during childbirth delivery.

FIG. 3 shows tensile stresses and tissue stretching experienced by the perineum during childbirth delivery.

FIG. 4 illustrates a perineal laceration in a closed-up front view of the perineum or the perineal plane view.

FIG. 5 illustrates a third or fourth degree perineal laceration that is initiated by a midline or medial episiotomy.

FIG. 6 shows the midline or medial cross-section of the perineum along the sagittal plane and direction of occasionally applied forces by medical staff.

FIG. 7 shows an embodiment of the present invention in preventing or limiting perineal laceration from advancing toward the anus.

FIG. 8 illustrates one possible support mechanism for using a topical patch.

FIGS. 9A and 9B show the front view and the midline or medial cross-sectional view of a hemi-circular topical patch of the present invention, as applied on a patient.

FIGS. 10A and 10B show the front view and the midline or medial cross-sectional view of a substantially square topical patch of the present invention, as applied on a patient.

FIG. 11 shows an elongated topical strip adhesively secured to the lower or posterior part of the perineal area between the vagina and the anus.

FIG. 12 shows an irregular-shaped topical patch adhesively secured to part of the perineal area.

FIG. 13 shows a square topical patch with a square reinforcement layer adhesively secured to part of the perineal area.

FIG. 14 shows a heart-shaped topical patch with a circular reinforcement layer adhesively secured to the perineal area.

FIGS. 15A through 15H show topical patches with a variety of regular geometric shapes with or without an opening.

FIGS. 16A through 16H show topical patches with a variety of star-like shapes with or without an opening.

FIGS. 17A through 17H show topical patches with a variety of irregular geometric shapes.

FIGS. 18A through 18H show topical patches with a variety of additional geometric shapes.

FIGS. 19A through 19H show examples of a topical device comprising an adhesive layer and a support layer, wherein the support layer may have a variety of material compositions, patterns or reinforcements.

FIGS. 20A through 20H show examples of a topical device comprising an adhesive layer and a support layer, wherein the support layer may have a variety of shapes, material compositions, patterns or reinforcements.

FIGS. 21A through 21H show examples of a topical device comprising an adhesive layer and a support layer, wherein the support layer may have a variety of shapes, material compositions, patterns or reinforcements.

FIGS. 22A through 22F show examples of the cross-section of a topical device comprising a variety of support layers, adhesive layers and release liners.

FIGS. 23A through 23F show examples of the cross-section of a topical device comprising a variety of reinforcements, support layers, adhesive layers and release liners.

FIGS. 24A through 24F show examples of the cross-section of a topical device comprising a variety of reinforcements, support layers, adhesive layers and release liners.

FIGS. 25A through 25D show examples of the cross-section of a non-planar topical device comprising a variety of support layers, adhesive layers and release liners.

FIGS. 26A through 26H show the top view of a topical device that is made of an oval-shaped adhesive patch reinforced with a circular patch.

FIGS. 27A through 27H show the top view of a topical device that is made of a relatively rectangular adhesive patch with slightly concave edges and reinforced with a circular patch.

FIGS. 28A through 28H show the top view of a topical device that is made of an elongated adhesive patch with two slightly concave edges and reinforced with a circular patch.

FIGS. 29A through 29D show the top view of a topical device that is made of a relatively square adhesive patch with two slightly concave edges and reinforced with a circular patch.

FIGS. 30A through 30C show the top view of a topical device with drainage tubing for anal discharge.

FIGS. 31A and 31B show the top view of a topical device with extended corners or cut-out channel for improved attachment.

FIGS. 32A and 32B show the front view of a topical patch with extended corners or cut-out channel as applied on a patient.

FIG. 33 shows the front view of an oval-shaped topical patch as applied on a patient.

FIG. 34 shows the front view of a relatively rectangular topical patch with concave edges as applied on a patient.

FIG. 35 shows the front view of an elongated topical patch with two concave edges as applied on a patient.

FIG. 36 shows the front view of a relatively square topical patch as applied on a patient.

FIGS. 37A and 37B show the front and the medial cross-sectional view, respectively, of a relatively squared topical patch with connected tubing and collection bag as applied on a patient.

FIGS. 38A through 38D show the pictures of a prototype topical patch and a test model used in the first experiment.

FIGS. 39A through 39D show the pictures of a prototype topical patch and a test model used in the second experiment.

FIGS. 40A and 40B show examples of a flexible topical patch having a support frame or support element.

### DETAILED DESCRIPTION OF THE INVENTION

For the purposes of promoting an understanding of the principles of the invention, reference will now be made to preferred embodiments and specific language will be used to describe the same. It will nevertheless be understood that no limitation of the scope of the invention is thereby intended. Alterations, modifications of the invention, and further applications of the principles of the invention as illustrated herein, are contemplated as would normally occur to one skilled in the art to which the invention relates.

As used herein, the term “tear-resistant” means capable of resisting significant stress and/or deformation without experiencing loss of integrity.

As used herein, the term “stretchable” means capable of being stretched or expanded in at least a dimension more than 50% of the non-stretched or non-expanded dimension without irreversible damage or loss of integrity to a device or material. The term “semi-stretchable” means capable of being stretched or expanded in at least a dimension up to 50% of the non-stretched or non-expanded dimension without irreversible damage or loss of integrity to a device or material. The term “substantially non-stretchable” means capable of being stretched or expanded in at least a dimension up to 20% of the non-stretched or non-expanded dimension without irreversible damage or loss of integrity to a device or material.

In general, the present invention provides topical devices for assisting the performance of a surgical incision and methods of making and using the same. A topical device of the present invention comprises a flexible patch that comprises a tear-resistant support layer and an adhesive layer disposed thereon.

In one aspect, a topical device of the present invention further comprises a high tear-resistant reinforcement layer or member disposed on the support layer, within the support layer, or between the adhesive layer and the support layer.

In another aspect, a topical device of the present invention is adhesively attached, affixed, bonded, or secured to a tissue, adjacent to an incision that was or will be made in the tissue, wherein at least a portion of the topical device is disposed substantially nonparallel to the incision.

In one embodiment, such reinforcement members can comprise deformable fibers, filaments, yarns, threads, wires, cables, tethers, strips, or bars that are made of polymeric, ceramic, metallic materials, or their composites.

In another embodiment, such reinforcement members can be imbedded within a matrix material, such as a polymeric material. The reinforcement members can be arranged in parallel or in the form of a mesh. Preferably, the reinforcement members are arranged in the matrix in the form of a mesh.

In still another aspect, a topical device of the present invention comprises a support layer and an adhesive layer disposed on one surface of the support layer, wherein the support layer comprises a plurality of reinforcement members disposed therein.

In still another aspect, the present invention provides topical devices and methods for adhesively attaching, affixing, bonding, or securing such devices onto the perineum of a patient undergoing vaginal childbirth delivery in order to reduce, limit or prevent perineal laceration and other tissue damages.

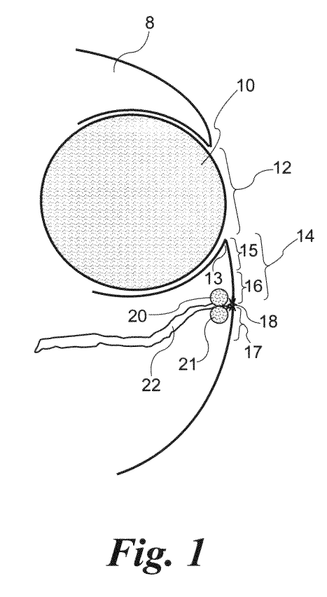

Reference now is made to FIG. 1 that reveals the midline or medial cross-section of the perineum along the sagittal plane as the patient is lying down on her back during childbirth delivery. The top of the baby's head 10 is revealed through the dilated vagina 12, which is located below the mons pubis 8 but above the anus 18. The perineal tissues located at or beneath the area 14 are prone to tear or laceration due to excessive stretching during the childbirth labor process. Laceration or episiotomy incision may be initiated at or near the section 13 of the dilated vagina 12 and propagates or advances toward the anus 18. The risk of third or fourth degree laceration becomes significant as laceration advances toward the anus into the perineal tissues approximately at or beneath the area 16, under which the anal sphincter muscle 20 and the rectum 22 are located.

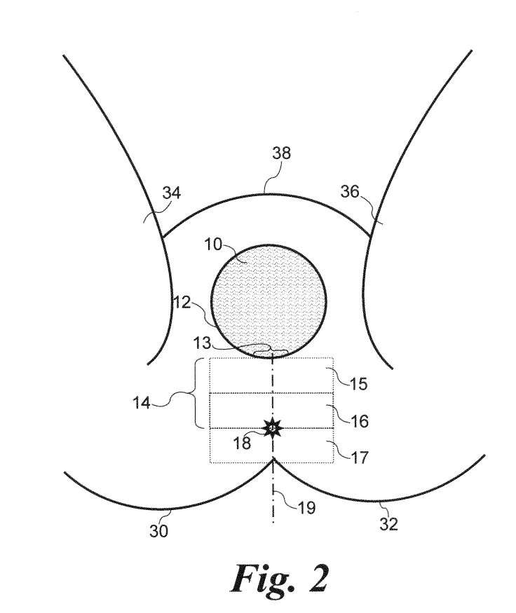

FIG. 2 shows the front view of the perineum that is substantially transverse to the sagittal plane as the patient is lying down on her back during childbirth delivery. As the patient spreads her left thigh 36 and right thigh 34, her bulging abdomen 38 can be seen in this view. The top of the baby's head 10 is revealed through the dilated vagina 12, which is located above the anus 18. The perineum, the perineal areas or the perineal tissues referred to in this document include approximately the area 15 right below the vagina, the area 16 right above the anus and the area 17 right below the anus. The perineal tissues between the vagina and the anus located approximately at or beneath the area 14, which consists of approximately the areas 15 and 16, are prone to tear or laceration due to excessive stretching during the childbirth labor process. Laceration or episiotomy incision may be initiated at or near the section 13 of the dilated vagina 12 and propagates or advances toward the anus 18, commonly along or near the midline 19. The risk of third or fourth degree laceration becomes significant as laceration advances toward the anus into the perineal tissues approximately at or beneath the area 16.

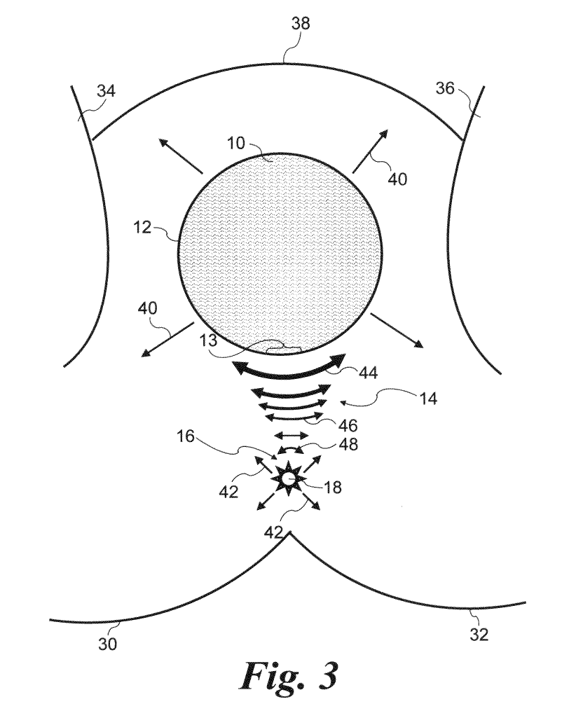

As shown in a closed-up perineal front view by FIG. 3, the perineum may experience significant tensile stresses and tissue stretching that substantially transverse to the sagittal plane while the patient is lying down on her back and undergoing childbirth delivery. As the patient spreads her left thigh 36 and right thigh 34, her bulging abdomen 38 is shown in this view. During this stage of delivery, the top of the baby's head 10 is revealed through the dilated vagina 12. The pushing action along the sagittal plane and the continuing dilation in the direction 40 of the dilated vagina 12 may cause tensions 44 and 46 in the perineal tissues 14. The tensions 44 and 46 essentially occur along a plane that is tangent or parallel to the perineum, which is substantially transverse to the sagittal plane. This plane will be referred to as the perineal plane in this document. The pushing action may also result in straining of the perianal tissues and dilation of the anus in the direction 42. As a result, the perineal tissues 16 right above the anus 18 may experience tension 48.

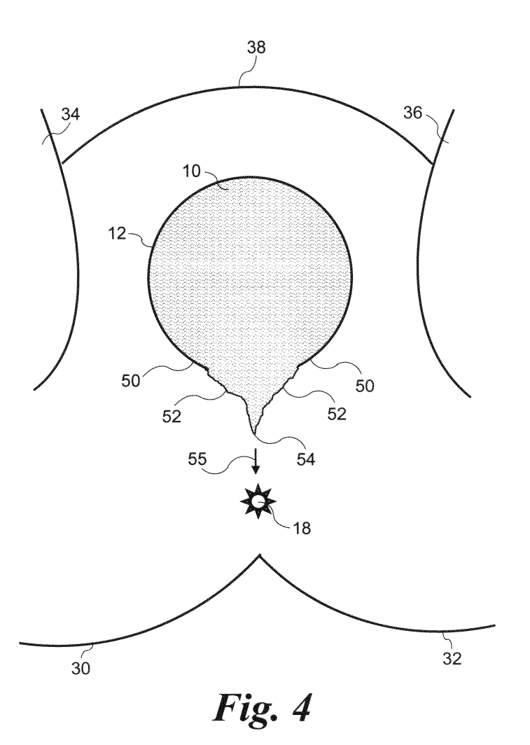

FIG. 4 illustrates a perineal laceration in a closed-up front view of the perineum or the perineal plane view. During this stage of delivery, the top of the baby's head 10 is revealed through the dilated vagina 12. The pushing action along the sagittal plane and the continuing dilation of the dilated vagina 12 along the perineal plane may cause a midline or medial laceration 52 with the potential propagation or advancement 55 of the leading edge 54 toward the anus 18.

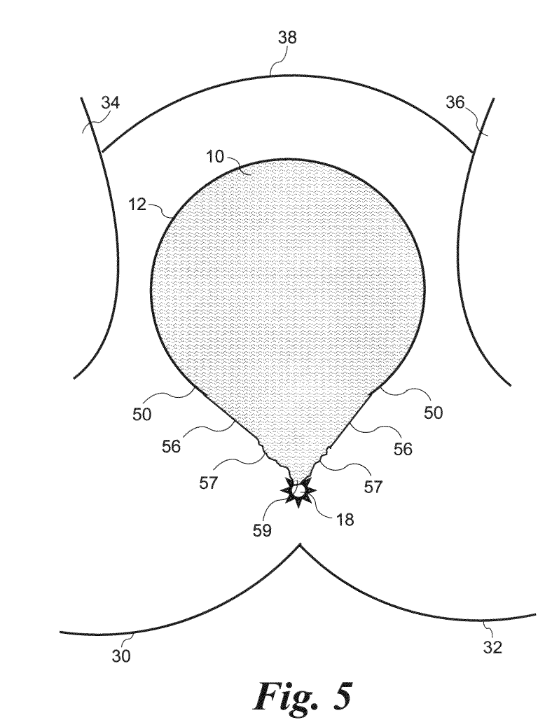

Also in the perineal plane view, FIG. 5 illustrates a third or fourth degree perineal laceration that is initiated by a midline or medial episiotomy. During this stage of delivery, the top of the baby's head 10 is revealed through the dilated vagina 12. A midline or medial episiotomy cut 56 may be performed to facilitate the delivery process. The pushing action along the sagittal plane and the continuing dilation of the dilated vagina 12 along the perineal plane may cause the midline or medial episiotomy 56 to spread apart and advance toward the anus 18, which may result in a third or fourth degree perineal laceration 57 with the leading edge 59 reaching the anus 18. In the case of a third degree laceration, the anal sphincter is typically damaged or affected by the tear. In the case of a fourth degree of laceration, the rectal lining is also damaged or affected by the tear.

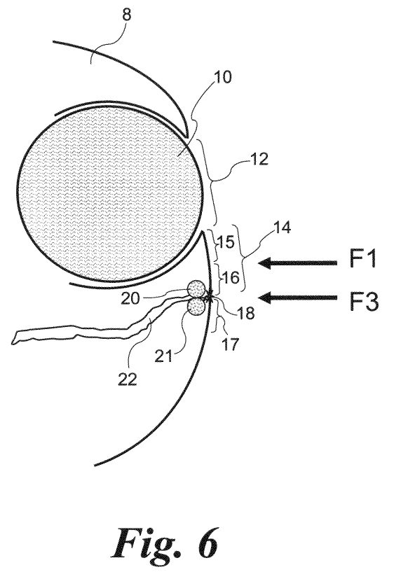

Referring now to FIG. 6 that shows the midline or medial cross-section of the perineum along the sagittal plane as the patient is lying down on her back during childbirth delivery. The pushing action by the patient during childbirth delivery can cause significant forces or pressure in tissues within or adjacent to the perineal area 14, such as the perineal tissues, anal sphincter muscles 20, the anus 18, the perianal tissues. Consequently, the tissues can undergo excessive straining that may lead to tissue damages or trauma including perineal laceration, strained perineal tissues, strained anal sphincters, anal tears, anal fissures, external hemorrhoid, internal hemorrhoid, prolapsed internal hemorrhoid, etc. Attempts have previously taken to reduce or prevent perineal laceration or other tissue damages by supporting or pushing against perineum 14 with a compressive force F1 or F3 along the sagittal plane. Hand support of the perineum 14 during delivery has been reported in the literature. Regardless of such attempts, there is presently no proven reliable method or device for preventing laceration or other tissue damages during childbirth labor. It may be argued that excessive compressive force F1 or F3 along the sagittal plane either by hand or by using a device may adversely affect the perineal tissues. Furthermore, excessive pushing or compression along the sagittal plane against the tissues in the perineal areas 14 close to the vagina 12 may interfere or adversely affect the delivery process or the baby.

FIG. 7 illustrates an embodiment of the present invention in preventing or limiting perineal laceration from advancing toward the anus. In this embodiment, the topical device is a substantially circular and flexible topical patch 60 having an opening 62 surrounded by the tear-resistant material of the support layer. The outer dimension (e.g., diameter) of the topical patch 60 may range from 10 mm to 100 mm, preferably from 20 mm to 90 mm, and more preferably from 30 mm to 70 mm. The dimension (e.g., diameter) of the central opening 62 may range from 1 mm to 60 mm, preferably from 5 mm to 50 mm, more preferably from 10 mm to 30 mm. The topical patch preferably has a substantially uniform thickness in the range between 0.1 mm and 10 mm, more preferably between 0.25 mm and 5 mm, and most preferably between 0.5 mm to 2.5 mm. The topical patch is adhesively attached, affixed, bonded, or secured to the perineum and covers part of the perineal area 16 (described in FIG. 2) above the anus 18 and the perineal area 17 (described in FIG. 2) below the anus 18. The topical patch has an appropriate structure and material composition that help the underlying tissues to resist tension, stretching, straining, or tear. Although the topical device may be flexible, semi-flexible or semi-rigid, the topical patch in this example is preferred to be substantially flexible or conformable. The topical device may be elastic, semi-elastic or inelastic. The topical device may also be stretchable, semi-stretchable or substantially non-stretchable. However, the topical patch in this example is preferably semi-stretchable or substantially non-stretchable depending on a variety of factors including the orientation of the support woven fabric in the patch relative to laceration. The patch may have printed information on the outer surface that may be useful for the users such as orientation, dimensions, instructions of use, warnings, etc. The topical patch shown in FIG. 7 comprises a support layer preferably made of a tear-resistant woven fabric and a pressure sensitive adhesive layer preferably made of an acrylate-based adhesive material. When adhered, attached, affixed or adhesively secured to the perineum, the patch may provide countering forces or stresses 66 to help the underlying perineal tissues to resist tension or tensile stresses 46 occurring along the perineal plane defined above. The patch may also provide countering forces or stresses 64 to help the underlying perineal or perianal tissues to resist radial expansion forces or stresses 42. In general, the patch 60 may provide support to the underlying perineal tissues against excessive stretching or expansion along the perineal plane that may result in perineal laceration, excessive straining of perineal and perianal tissues, or other damages to perineal and perianal tissues.

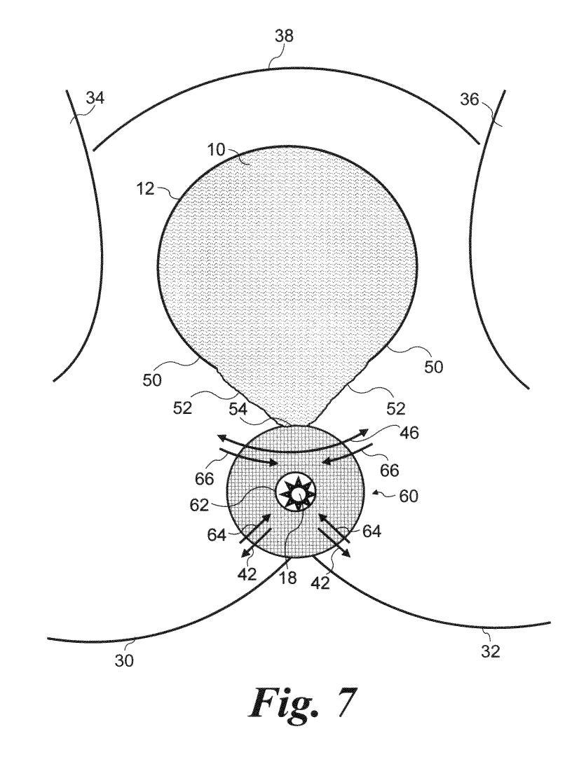

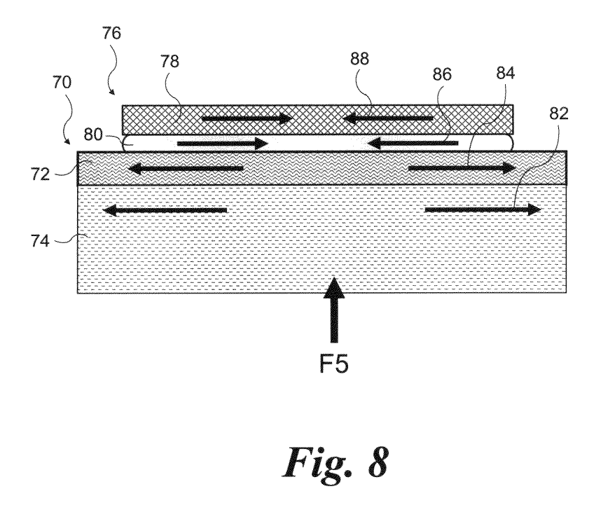

One possible support mechanism for using the topical patch discussed above may be further illustrated by FIG. 8. The pushing actions of the patient during delivery may result in force F5 substantially along the sagittal plane, which may partially lead to tensions or tensile stresses 82 and 84 along the perineal plane, which is substantially transverse to the sagittal plane. As a result, perineal dermal tissues may experience stretching along the same plane as the tensile stresses 82 and 84. Excessive tensile stresses and stretching may result in laceration or other tissue damages to the perineum. When the topical patch 76 is adhesively bonded or secured to the underlying dermal tissues 70, the tensile stresses 82 and 84 may be partially transferred to the support layer 78 through the adhesive layer 80. The support layer may be made of a material that is substantially non-stretchable and with appropriate mechanical properties. Examples of the support layer include bi-axially oriented polymer film, a woven fabric, a fiber or filament-reinforced polymer sheet, etc. The support layer may resist the transferred tensile stresses and the stretching when it is placed in tension with a countering tensile stress 88 through a shear stress 86 in the adhesive layer. This support mechanism may reduce, limit or prevent the underlying tissues 70 from experiencing excessive stretching that may lead to laceration and other tissue damages.

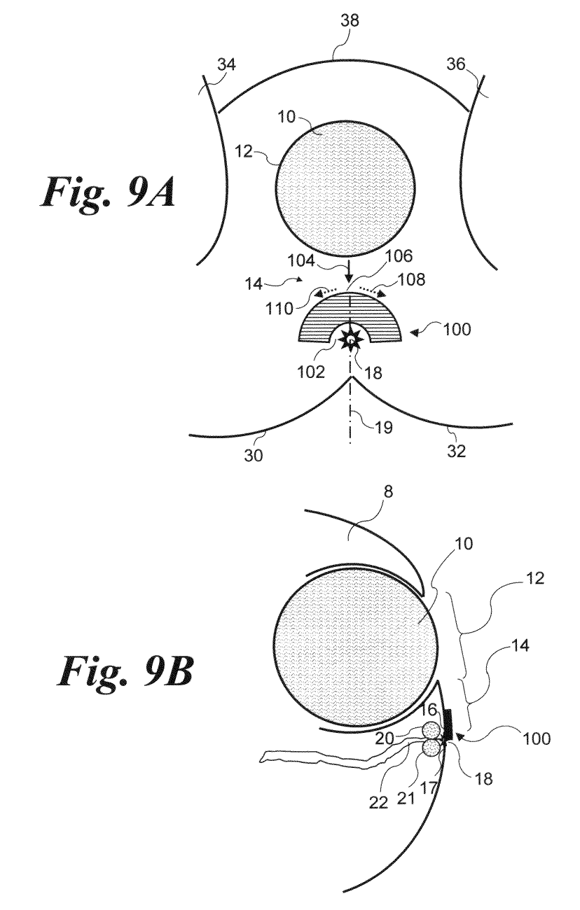

FIG. 9A shows a hemi-circular topical patch 100 adhesively secured to part of the perineal area 14 that may limit or prevent a laceration 104, which includes a stress- or strain-induced tear, an episiotomy-induced tear, and an episiotomy incision, from advancing toward the anus 18 when it reaches the patch 100 near the area 106. Alternately, the patch 100 may divert, deflect or redirect a medial laceration 104 toward a mediolateral direction 108 and 110. The hemi-circular topical patch 100 having a recess 102 is adhesively bonded or secured to the perineal area between the vagina 12 and the anus 18. In one embodiment, the topical patch is preferably substantially flexible or conformable and preferably semi-stretchable or substantially non-stretchable. In another embodiment, the topical patch may be semi-flexible and semi-stretchable. In yet another embodiment, the topical patch may be substantially rigid and substantially non-stretchable. The patch shown in FIG. 9A comprises a support outer layer preferably made of a tear-resistant material including a woven fabric and a reinforced polymer sheet, and a pressure sensitive adhesive layer preferably made of an acrylate-based adhesive material. Since the hemi-circular patch 100 shown in FIG. 9A has a shape that is half of the circular patch 60 shown in FIG. 7, the diameter and thickness ranges provided for the patch 60 are also applicable for the patch 100. It is preferred that the woven fabric or the reinforced polymer sheet used in the support layer has at least some fibers, filaments or elongated reinforcement elements orientated substantially perpendicular to the midline 19 connecting the vagina and the anus. When bonded to the perineum, the patch 100 may provide support to the underlying perineal tissues against excessive stretching and potential laceration near the anus 18.

FIG. 9B shows the sagittal midline cross-section of the perineum shown in FIG. 9A, which reveals the thickness or profile of the hemi-circular topical patch 100. It is preferred that the patch thickness or profile is smaller than its length, width, or diameter. In general, it is preferred that the largest dimension of the patch along the sagittal plane is smaller than its largest dimension along the perineal plane, which is defined in this document as the plane parallel or tangent to the perineum. As shown, the patch 100 may be located on the lower part of the perineal area 14 between the vagina and the anus, right above the anus 18, and relatively adjacent to the underlying anal sphincter 20. Thus, the patch 100 may not interfere or affect the upper part of the perineal area 14 as this part of the perineum may need to undergo stretching, laceration or even episiotomy in order to facilitate delivery. By locating right above the anus 18 of the perineal area 14, the patch 100 may help limit or prevent a laceration from advancing toward or reaching the anus 18, which may cause damage or injury to the anal sphincter 20 or the rectal mucosa 22. Alternately, the patch 100 may divert or redirect a medial laceration toward a mediolateral direction, which may reduce the risk of damage or injury to the anal sphincter 20 or the rectal mucosa 22.

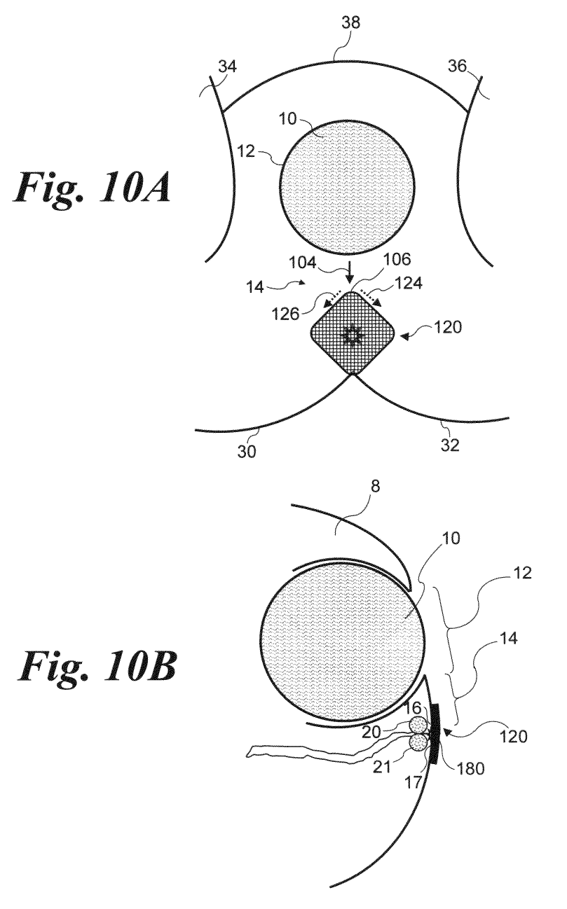

FIG. 10A shows a substantially square topical patch 120 adhesively secured to part of the perineal area 14 between the vagina and the anus, the perineal area covering perianal tissues, and the anus. The patch 120 may limit or prevent a laceration 104, which includes a stress- or strain-induced tear, an episiotomy-induced tear, and an episiotomy incision, from advancing toward the anus when it reaches the patch 120 near the area 106. Alternately, the patch 120 may divert or deflect a medial laceration 104 toward a mediolateral direction 124 and 126. The squared topical patch 120 without a central opening is adhesively bonded or secured to the perineal area around the anus and covers the anus. The topical patch is preferably substantially flexible and preferably semi-stretchable or substantially non-stretchable. The side of the square patch 120 preferably has a dimension in the range between 10 mm and 100 mm, more preferably between 20 mm and 90 mm, and most preferably between 30 mm and 70 mm. The topical patch preferably has a substantially uniform thickness in the range between 0.1 mm and 10 mm, more preferably between 0.25 mm and 5 mm, and most preferably between 0.5 mm to 2.5 mm. The patch may have information printed on the outer surface that may be useful to the users such as references for orientation, dimensions, instructions for use, warnings, etc. The patch comprises a support layer preferably made of a polymeric film laminated with a woven fabric and a pressure sensitive adhesive layer preferably made of a rubber-based adhesive material. In one embodiment, it is preferred that the woven fabric has at least some filaments oriented substantially perpendicular to the midline connecting the vagina and the anus for reduced stretchability. In another embodiment, it is preferred that the woven fabric has the filaments oriented approximately 45 degrees relative to the midline connecting the vagina and the anus to allow some stretchability. When adhesively bonded to the perineum, the patch 120 may provide support to the underlying perineal tissues against excessive stretching, which may result in perineal laceration, excessive perineal tissue straining, excessive perianal tissue straining, or anal tissue extrusion through the anal aperture.

FIG. 10B shows the sagittal midline cross-section of the perineum shown in FIG. 10A, which reveals the thickness or profile of the square topical patch 120. It is preferred that the patch thickness or profile along the sagittal plane is smaller than its length, width, or diameter along the perineum plane. In general, it is preferred that the largest dimension of the patch along the sagittal plane is smaller than its largest dimension along the perineum plane. As shown, the patch 120 may be located both above and below the anus 18 and relatively adjacent to the underlying anal sphincter 20 and 21. Thus, the patch 120 may not interfere or affect the upper part of the perineal area 14 as this part of the perineum may need to undergo stretching, laceration or even episiotomy in order to facilitate delivery. By locating around and at the anus 18, the patch 120 may help prevent a laceration from advancing toward or reaching the anus 18, which may cause damage or injury to the anal sphincter 20 or the rectal mucosa 22. Alternately, the patch 120 may divert a medial laceration toward a mediolateral direction, which may reduce the risk of damage or injury to the anal sphincter 20 or the rectal mucosa 22. As the anus is surrounded and covered by the patch 120, support may be provided to the underlying perineal tissues against excessive stretching or dilation, which may results in perineal laceration, excessive perineal tissue straining, excessive perianal tissue straining, or anal tissue extrusion through the anal aperture.

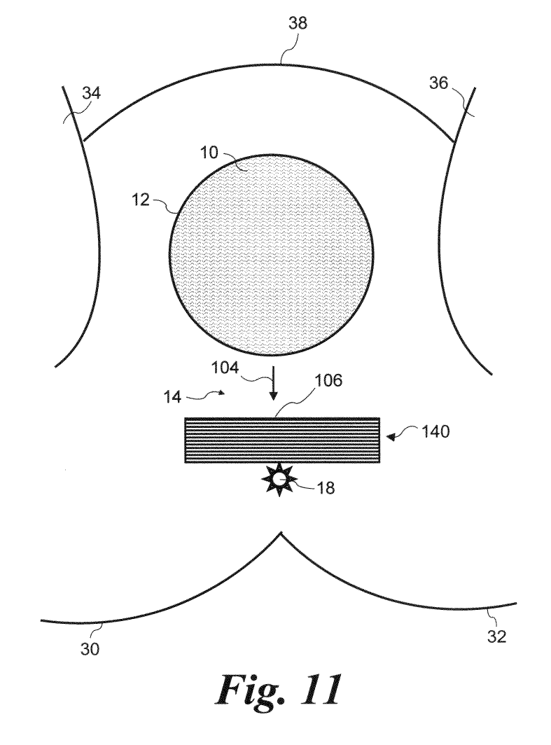

FIG. 11 shows an elongated topical strip 140 adhesively secured to the lower or posterior part of the perineal area 14 between the vagina and the anus. The patch 140 may limit or prevent a laceration 104, which includes a stress- or strain-induced tear, an episiotomy-induced tear, and an episiotomy incision, from advancing toward the anus when it reaches the patch 140 near the area 106. The patch 140 may divert or deflect a medial laceration 104 toward a lateral direction along its edge or toward a mediolateral direction. The patch 140 may also reduce, limit or prevent a mediolateral tear or incision from progressing when it reaches the elongated edge of the patch. The topical strip 140 may optionally have a wider width and may cover part or all of the perianal area or the anus. The topical strip may have a length in the range between 10 mm and 300 mm, preferably between 20 mm and 200 mm, and more preferably between 30 and 150 mm. The topical strip may have a width in the range between 2 mm and 80 mm, preferably between 5 mm and 50 mm, and more preferably between 10 and 30 mm. The topical patch preferably has a substantially uniform thickness in the range between 0.1 mm and 10 mm, more preferably between 0.25 mm and 5 mm, and most preferably between 0.5 mm to 2.5 mm. The topical strip is preferably substantially flexible and preferably substantially non-stretchable. The strip consists of a support layer preferably made of a polymeric film reinforced with elongated fibers or filaments and a pressure sensitive adhesive layer preferably made of a modified acrylate-based adhesive material. It is preferred that at least some of the fibers, filaments or elongated reinforcement elements in the support layer have an orientation substantially perpendicular to the midline connecting the vagina and the anus. When adhesively bonded to the perineum, the strip 140 may provide support to the underlying perineal tissues against excessive stretching, which may results in perineal laceration, excessive perineal tissue straining, excessive perianal tissue straining, or anal tissue extrusion through the anal aperture.

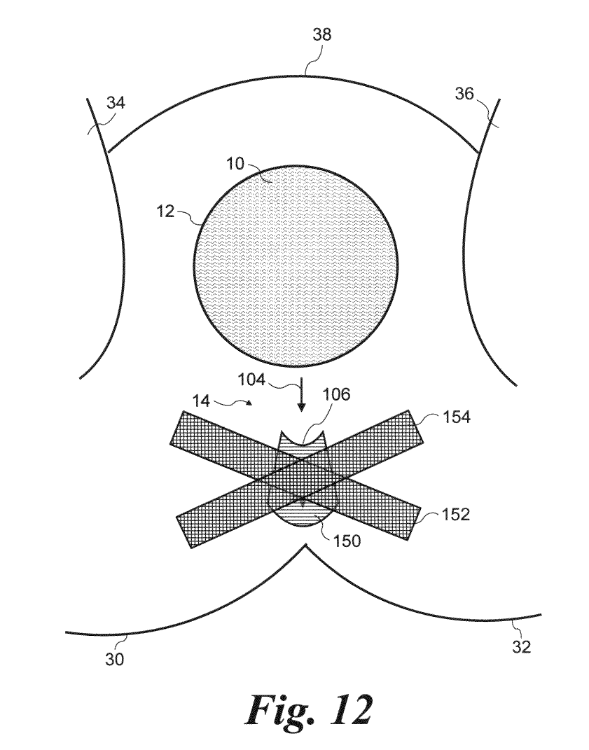

FIG. 12 shows an irregular-shaped topical patch 150 adhesively secured to part of the perineal area 14 between the vagina and the anus, the perineal area covering perianal tissues, and the anus. The topical patch 150 has a concave edge near the area 106 and faces the dilated vagina 12. The patch 150 may limit or prevent a laceration 104, which includes a stress- or strain-induced tear, an episiotomy-induced tear, and an episiotomy incision, from advancing toward the anus when it reaches the patch 150 near the area 106. It may be possible that the patch 150 may divert or deflect a medial laceration 104 toward a different direction. The topical patch 150 may optionally have additional fixation elements such as adhesive tapes 152 and 154. Adhesive tapes 152 and 154 may be integral parts of the patch 150 or may be provided separately as components in a product kit which includes the patch. The topical patch is preferably substantially flexible and preferably substantially non-stretchable. The patch consists of a support layer preferably made of a non-woven mesh wherein the fibers have been thermally fused and a pressure sensitive adhesive layer preferably made of a modified acrylate-based adhesive material. When adhesively bonded to the perineum, the patch 150 may provide support to the underlying perineal tissues against excessive stretching, which may results in perineal laceration, excessive perineal tissue straining, excessive perianal tissue straining, or anal tissue extrusion through the anal aperture.

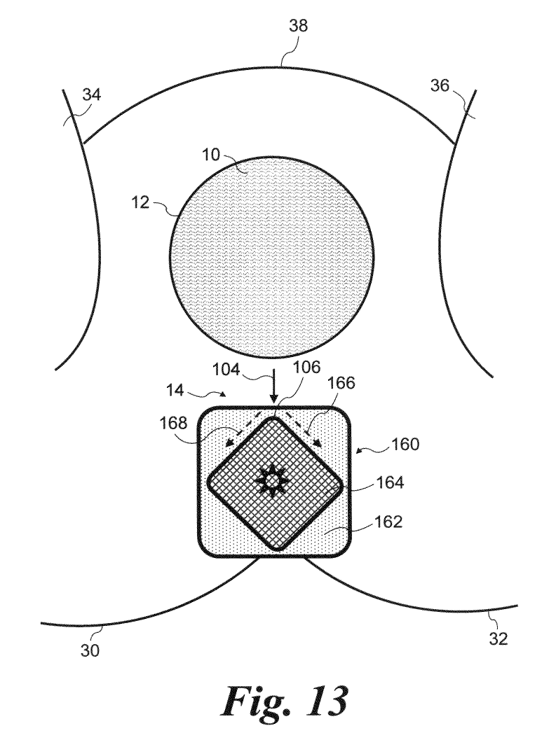

FIG. 13 shows a square topical patch 160 adhesively secured to part of the perineal area 14 between the vagina and the anus, the perineal area covering perianal tissues, and the anus. The patch 160 may limit or prevent a laceration 104, which includes a stress- or strain-induced tear, an episiotomy-induced tear, and an episiotomy incision, from advancing toward the anus when it reaches the patch 160 near the area 106. Alternately, the patch 160 may divert, deflect or redirect a medial laceration 104 toward a mediolateral direction 166 and 168. It may be possible that laceration is diverted toward a different direction such as laterally along the outer edge of the patch. The patch 160 may also reduce, limit or prevent a mediolateral laceration or a mediolateral episiotomy-induced tear from progressing when it reaches the edge of the patch. The patch 160 without an optional central opening is adhesively bonded or secured to the perineal area around the anus and covers the anus. The topical patch is preferably substantially flexible and preferably partially non-stretchable. The topical patch 160 in FIG. 13 may have dimensions similar to those of the patch 120 shown in FIG. 12A. The support layer of the square topical patch 160 may be made of a composite or laminate of a substantially stretchable polymer film 162 and a substantially non-stretchable woven fabric 164. The adhesive layer of the patch 160 is preferably made of an acrylate-based or rubber-based adhesive material. When adhesively bonded to the perineum, the patch 120 may provide support to the underlying perineal tissues against excessive stretching, which may results in perineal laceration, excessive perineal tissue straining, excessive perianal tissue straining, or anal tissue extrusion through the anal aperture.

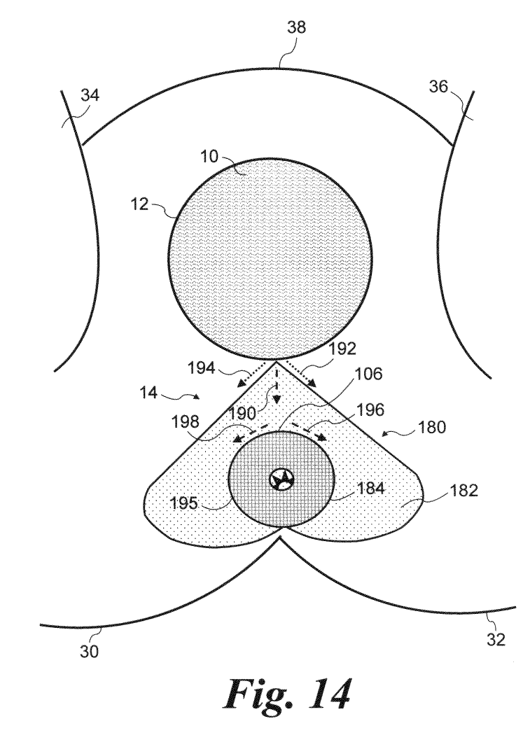

FIG. 14 shows a heart-shaped topical patch 180 adhesively secured to part of the perineal area 14 between the vagina and the anus, and the perineal area covering perianal tissues. The patch 180 may bias, divert, deflect or redirect an otherwise medial laceration toward a mediolateral direction 192 and 194. If a medial laceration 190 is formed and advanced through the pliable polymer layer 182, the patch 180 may reduce, limit or prevent the laceration 190 from advancing toward the anus when it reaches the edge of the reinforced area 184 of the patch 180 near the area 106. The patch 180 with an optional small central opening is adhesively bonded or secured to the perineal area around the anus and only partially covers the anus. The topical patch is preferably at least partially flexible and preferably partially non-stretchable. The support layer of the heart-shaped topical patch 180 is made of a composite or laminate of a substantially stretchable polymer film 182 and a tear-resistant woven fabric 184. The adhesive layer of the patch 180 is preferably made of an acrylate-based or silicone-based adhesive material. When adhesively bonded to the perineum, the patch 180 may provide support to the underlying perineal tissues against excessive stretching, which may results in perineal laceration, excessive perineal tissue straining, excessive perianal tissue straining, or anal tissue extrusion through the anal aperture.

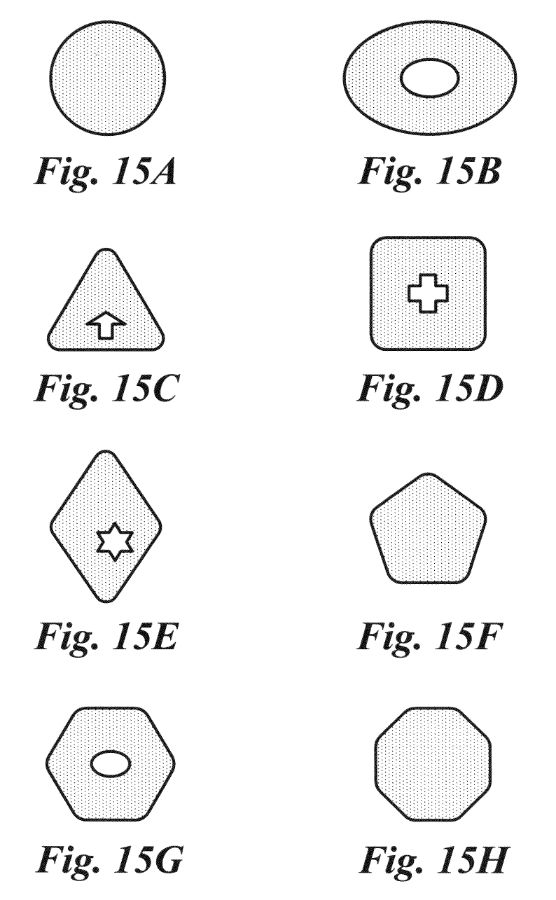

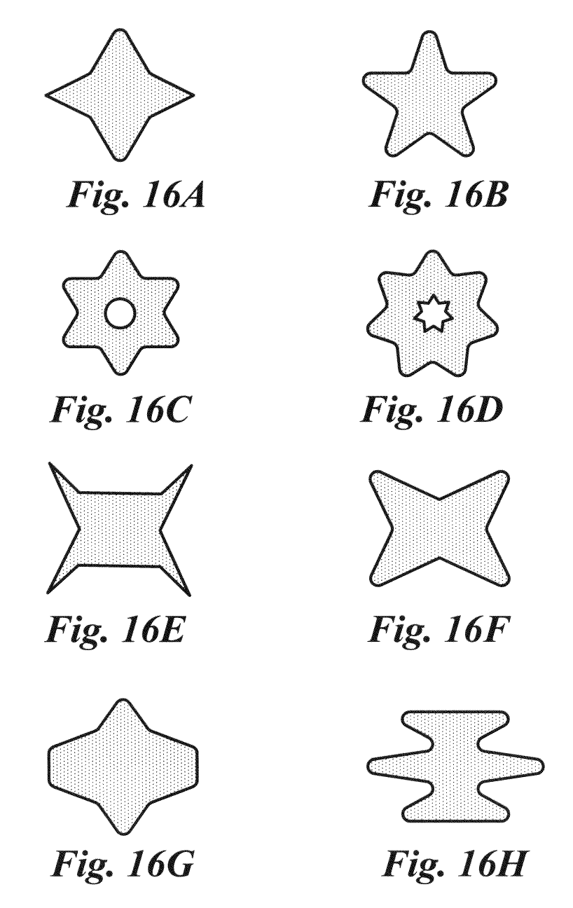

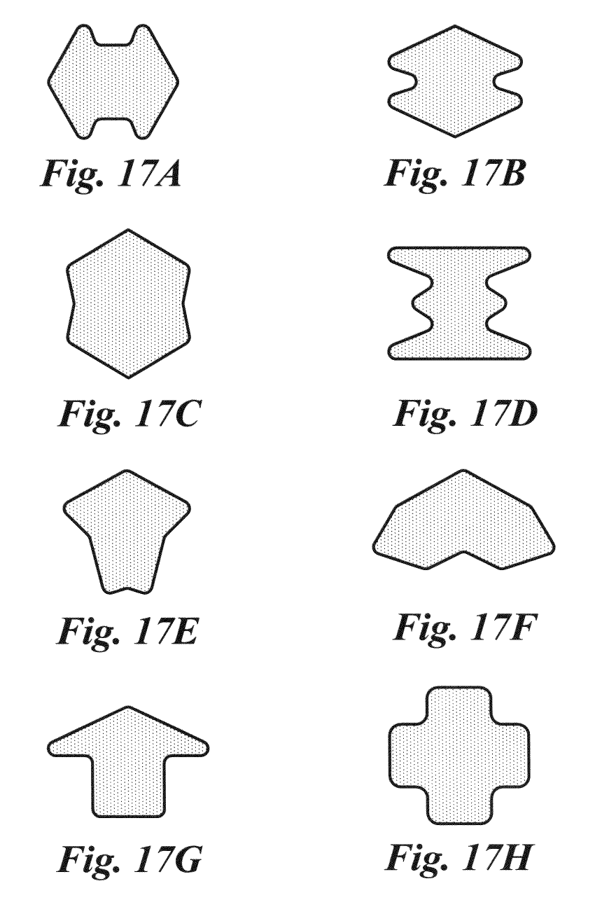

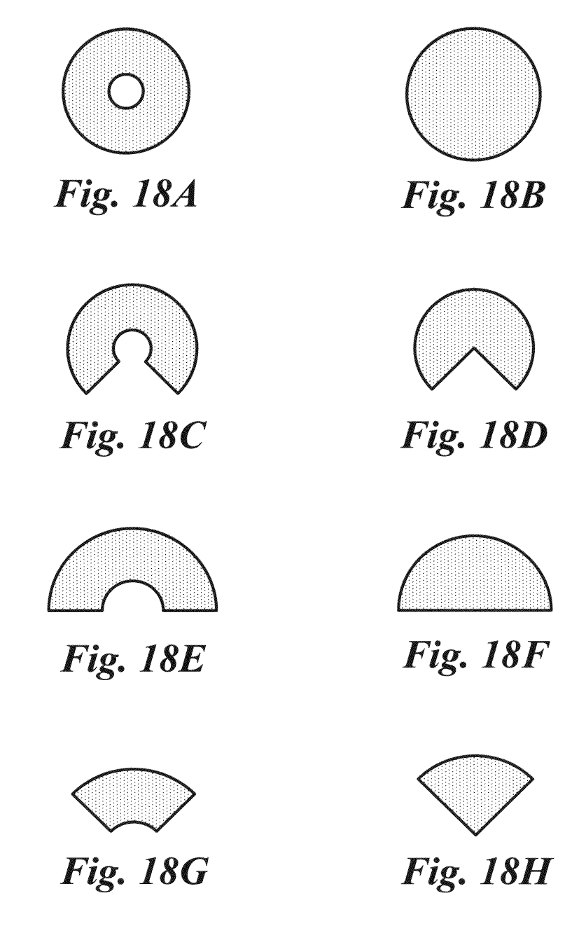

The topical devices or topical patches may have a variety of geometries or shapes which include regular geometric shapes as shown in FIG. 15A through FIG. 15H, star shapes as shown in FIG. 16A through FIG. 16H, and irregular geometric shapes as shown in FIG. 17A through FIG. 17H. The topical devices or patches may have an opening located either near the center as shown in FIGS. 15B, 15D and 15 G or away from the center as shown in FIGS. 15C and 15E. The topical device or patch may have a shape that is a portion or a section of a regular or irregular geometric shape. Examples of a device with a sectional shape are shown in FIGS. 18C, 18E and 18G. Additional examples of a device with a sectional shape are shown in are shown in FIGS. 18D, 18F and 18H.

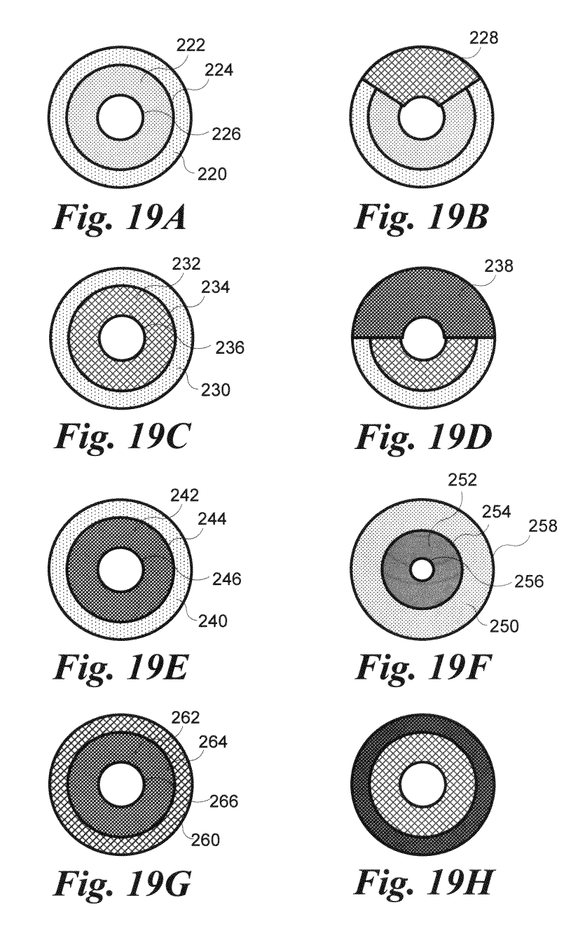

Although the topical device may have any shapes including those described above, examples concerning further details of the topical devices are provided below using a basic shape such as a ring shape or a round shape. It will nevertheless be understood that no limitation of the scope of the invention is thereby intended. The topical device may further be designed and manufactured such that it may have a variety of material compositions, a variety of patterns of material combination, lamination, reinforcement, etc. that are appropriate for the application described herein. FIG. 19 through FIG. 25 provide examples of a topical device comprising an adhesive layer and a support layer wherein the support layer may have a variety of material compositions or patterns.

FIG. 19A shows the top view of a substantially round and planar topical device that is made of two concentric rings of materials: a pliable polymer outer ring 220 and a substantially tear-resistant toughened polymer inner ring 222. There may be a gradual transition or blending near the demarcation 224 between the outer ring 220 and the inner ring 222. Alternately, the outer ring 220 may extend up to the inner edge 226 and the inner ring 222 is laminated on top of the outer ring 220. The topical device may be positioned and adhesively secured to the perineal area such that the anus approximates the central opening of the device. The toughened polymer inner ring 222 with substantial tear-resistance may reduce, limit or prevent a laceration or an episiotomy-induced tear from approaching or reaching the anus. The device may help divert, deflect or redirect a medial laceration or a medial episiotomy-induced tear toward a mediolateral direction, and thus, may reduce the risk of damage to the anal sphincter and the rectal mucosa. The device may also help reduce, limit or prevent other tissue damages by limiting excessive stretching.

FIG. 19B shows the top view of a similar device as FIG. 19A except the topical device is further reinforced with a tear-resistant woven fabric section 228. Reinforcement may be achieved via a variety of means including lamination, bonding, fusion, embedding, or combinations thereof. As the device is adhesively bonded or secured to the perineal area, it is preferred that the tear-resistant woven fabric section 228 is at least partially aligned with the midline connecting the vagina and the anus in order to reduce, limit or prevent a laceration or an episiotomy-induced tear from approaching or reaching the anus.

FIG. 19C shows the top view of a round and substantially planar topical device that is made of two concentric rings of materials: a substantially pliable polymer outer ring 230 and a tear-resistant woven fabric inner ring 232. There may be a gradual transition or blending near the demarcation 234 between the outer ring 230 and the inner ring 232. Alternately, the outer ring 230 may extend up to the inner edge 236 and the inner ring 232 is either laminated on top of the outer ring 230 or embedded within the pliable polymer of the outer ring 230. The topical device may be positioned and adhesively secured to the perineal area such that the anus approximates the central opening of the device. The tear-resistant woven fabric inner ring 232 may reduce, limit or prevent a laceration or an episiotomy-induced tear from approaching or reaching the anus.

FIG. 19D shows the top view of a similar device as FIG. 19C except the topical device is further reinforced with a high tear-resistant woven fabric section 238 in the upper half. Reinforcement may be achieved via a variety of means including lamination, bonding, fusion, embedding, weaving, knitting, or combinations thereof. As the device is adhesively bonded or secured to the perineal area, it is preferred that the high tear-resistant woven fabric section 238 is at least partially aligned with the midline connecting the vagina and the anus in order to reduce, limit or prevent a laceration or an episiotomy-induced tear from approaching or reaching the anus.

FIG. 19E shows the top view of a round topical device that is made of two concentric rings of materials: a pliable polymer outer ring 240 and a high tear-resistant woven fabric inner ring 242. There may be a gradual transition or blending near the demarcation 244 between the outer ring 240 and the inner ring 242. Alternately, the outer ring 240 may extend up to the inner edge 246 and the inner ring 242 is either laminated on top of the outer ring 240 or embedded within the pliable polymer of the outer ring 240. The topical device may be positioned and adhesively secured to the perineal area such that the anus approximates the central opening of the device. The high tear-resistant woven fabric inner ring 242 may reduce, limit or prevent a laceration or an episiotomy-induced tear from approaching or reaching the anus.

FIG. 19F shows the top view of a round and non-planar topical device that is made of two concentric sections of materials: a high tear-resistant rigid plastic section 252 and a substantially tear-resistant toughened polymer outer section 250. The rigid plastic section 252 may be laminated, bonded or thermally fused with the toughened polymer section 250. The device may be concave such that the inner edge 256 is below the outer edge 258 and the device may resemble a funnel shape. Alternately, the device may be convex such that the inner edge 256 is above the outer edge 258 and the device may resemble a cone shape. In other embodiments, one of the two sections can be planar while the other is not. It is preferred that the height or depth of the device measuring between the inner edge 256 and the outer edge 258 is smaller than the largest dimension of the device, which is the diameter of the outer edge 258. The topical device may be positioned and adhesively secured to the perineal area such that the anus approximates the central opening of the device. The toughened polymer outer ring 250 with a substantial tear resistance and the rigid plastic section 252 with high tear resistance may reduce, limit or prevent a laceration or an episiotomy-induced tear from approaching or reaching the anus.

FIG. 19G shows the top view of a round and slightly concave topical device that is made of two concentric rings of materials: a tear-resistant woven fabric outer ring 260 and a high tear-resistant woven fabric inner ring 262. There may be a gradual transition or blending near the demarcation 264 between the outer ring 260 and the inner ring 262. Alternately, the outer ring 260 may extend up to the inner edge 266 and the inner ring 262 is either laminated on top of the outer ring 260 or intertwined with the outer ring 260. The topical device may be positioned and adhesively secured to the perineal area such that the anus approximates the central opening of the device. The tear resistance of the two woven fabric rings 260 and 262 may reduce, limit or prevent a laceration or an episiotomy-induced tear from approaching or reaching the anus.

FIG. 19H shows the top view of a similar device as FIG. 19G except the two types of materials are reversed in locations. Alternately, the topical device may be slightly convex, partially convex, partially concave, wavy, or in any configuration that is essentially not planar.

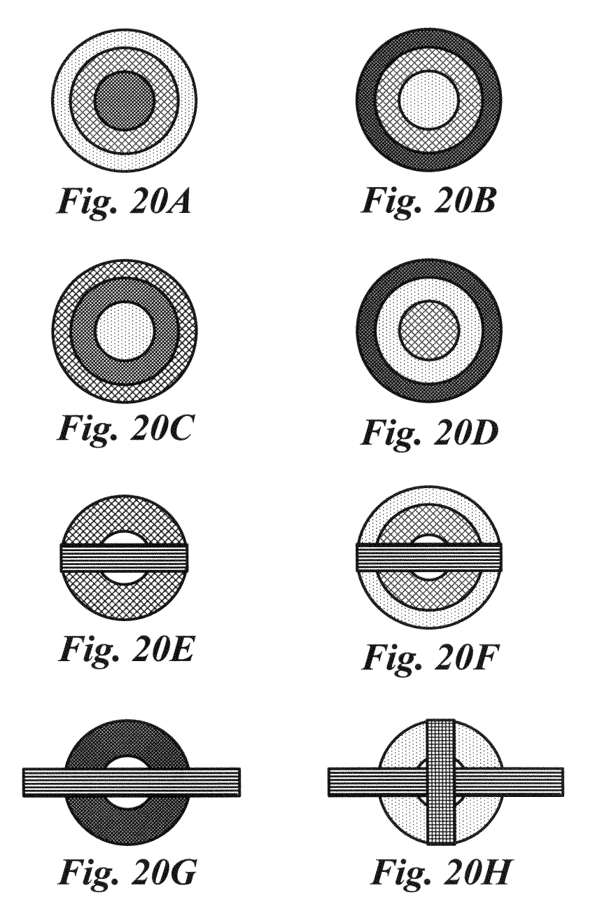

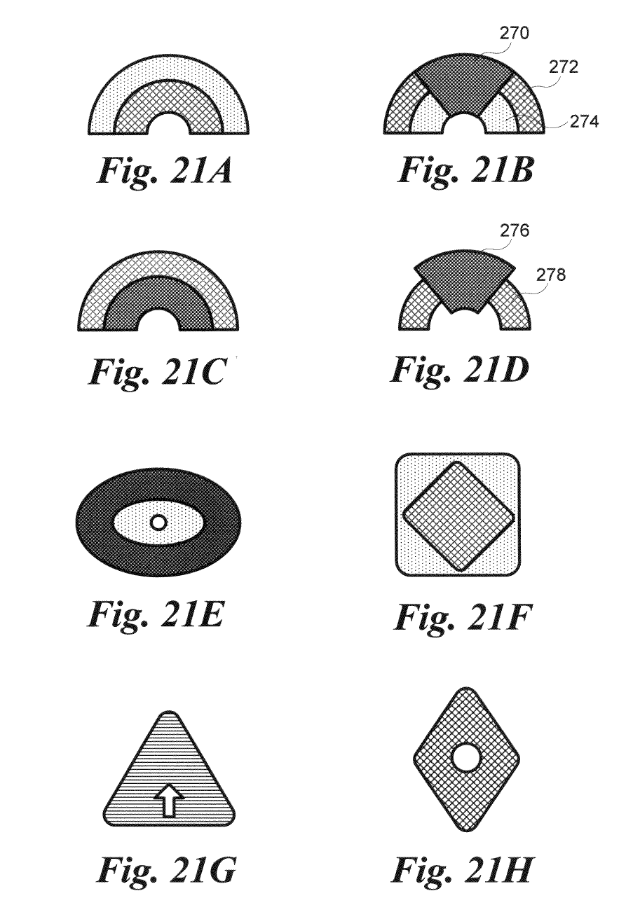

FIG. 20A is similar to FIG. 19C except the central opening is covered with a high tear-resistant woven fabric.

FIG. 20B is similar to FIG. 19H except the central opening is covered with a pliable polymer layer.

FIG. 20C is similar to FIG. 19G except the central opening is covered with a pliable polymer layer.

FIG. 20D shows the top view of a round topical device made of three different materials: a high tear-resistant woven fabric outer ring, a pliable polymer ring and a tear-resistant woven fabric covering the round central area. The topical device may be slightly convex, partially convex, slightly concave, partially concave, wavy, or in any configuration that is essentially not planar.

FIG. 20E shows the top view of a round topical device made of tear-resistant woven fabric ring reinforced with an elongated strip. The strip may optionally have fibers or filaments oriented along its length for reinforcement. The topical device may be positioned and adhesively secured to the perineal area such that the anus approximates the central opening of the device and the elongated strip is perpendicular to the midline connecting the vagina and the anus. The tear-resistant woven fabric ring reinforced with the elongated strip may reduce, limit or prevent a laceration or an episiotomy-induced tear from approaching or reaching the anus.

FIG. 20F is similar to FIG. 19C but with an elongated strip.

FIG. 20G shows the top view of a round topical device made of a high tear-resistant woven fabric ring reinforced with an elongated strip that extends substantially beyond the outer edge for additional fixation support.

FIG. 20H shows the top view of a round topical device made of a pliable polymer ring reinforced with an elongated strip that extends substantially beyond the outer edge for additional fixation support. The device is further reinforced with woven fabric tape and the tape is positioned perpendicular to the reinforcing elongated strip.

FIG. 21A shows the top view of a hemi-circular topical device with a similar material pattern as the device shown in FIG. 19C. The topical device may be positioned and adhesively secured to the perineal area between the vagina and the anus such that the anus approximates the central recess at the bottom of the device. The inner hemi-circular section made of tear-resistant woven fabric may reduce, limit or prevent a laceration or an episiotomy-induced tear from approaching or reaching the anus.

FIG. 21B shows the top view of a hemi-circular topical device with a middle section made of a high tear-resistant woven fabric 270, outer side sections 272 made of tear-resistant woven fabric and inner side sections 274 made of a pliable polymer. As the device is adhesively bonded or secured to the perineal area, it is preferred that the high tear-resistant woven fabric section 270 is at least partially aligned with the midline connecting the vagina and the anus in order to reduce, limit or prevent a laceration or an episiotomy-induced tear from approaching or reaching the anus.

FIG. 21C shows the top view of a hemi-circular topical device with a similar material pattern as FIG. 19G.

FIG. 21D shows the top view of a hemi-circular topical device made of a tear-resistant woven fabric with an enlarged laminated middle section made of a high tear-resistant woven fabric. The high tear-resistant middle section is preferably aligned with the midline between the vagina and the anus in order to reduce, limit or prevent a laceration or an episiotomy-induced tear from reaching the anus.

FIG. 21E shows the top view of an elliptical topical device with an outer ring made of a high tear-resistant woven fabric and a central elliptical section made of a pliable polymer. The device may be used in a vertical position (its major diameter parallel to the midline connecting the vaginal and the anus) or a horizontal position (its major diameter perpendicular to the midline connecting the vaginal and the anus) depending on the length of the perineum between the vagina and the anus or the doctor's preference. For example, if the patient has a small perineal length, the device may be preferred to be used in a horizontal position. If the patient has a large perineal length and the doctor prefers maximum protection, the device may be used in the vertical position. Further adjustment may also be made by the relative positioning of the central section against the anus.

FIG. 21F shows the top view of a square topical device made of a pliable polymer with a central square section reinforced with a woven fabric via lamination, bonding, fusion, embedding, or combinations thereof.

FIG. 21G shows the top view of a triangular topical device made of a polymeric film reinforced with elongated members, such as fibers, filaments or the alike, oriented in the horizontal direction. The device has an arrow-shaped opening located near the bottom of the patch for reference purpose or for alignment with the anus.

FIG. 21H shows the top view of a rhombus-shaped topical device made of a tear resistant woven fabric having a central circular opening. The device may be used in a vertical or a horizontal position depending on the length of the perineum between the vagina and the anus or the surgeon's preference. For example, if the patient has a small perineal length, the device may be preferred to be used in a horizontal position so that the highest corner of the device does not get too close to the vagina. If the patient has a large perineal length and the surgeon prefers maximum protection, the device may be used in the vertical position so that distance between the highest corner of the device and the anus or the central opening is maximized. Further adjustment may also be made by the relative positioning of the central opening against the anus.

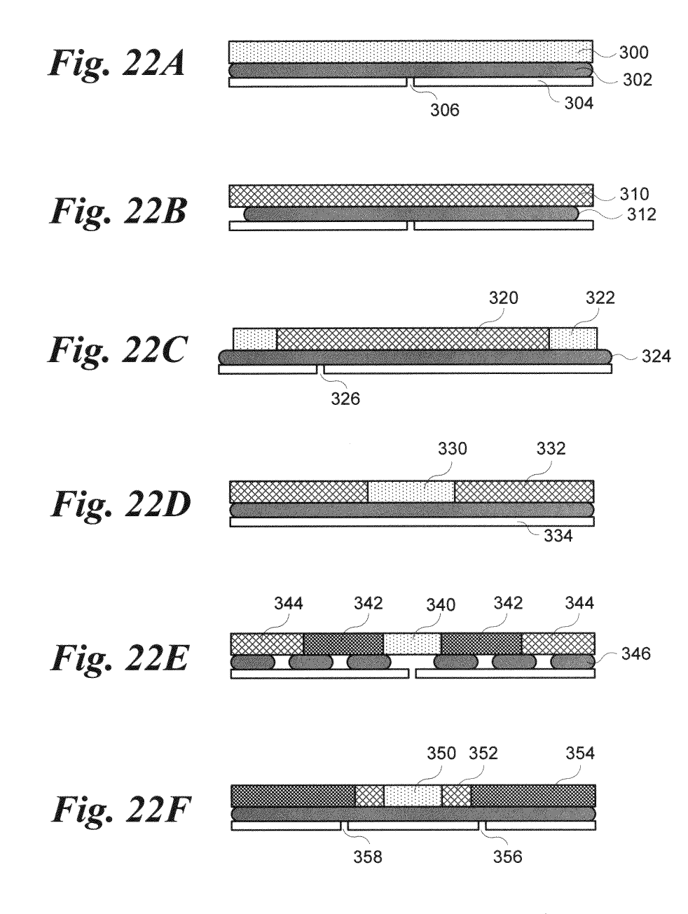

FIG. 22A shows the midline cross-section of a topical device with a support layer 300 made of a pliable polymer, an adhesive layer 302, and release liners 304. A slit 306 in the release liners 304 enables removal of the release liners 304 at the same time or in stages during application of the device.

FIG. 22B shows the midline cross-section of a topical device with a support layer 310 made of a tear-resistant woven fabric, an adhesive layer with recessed edge 312, and release liners.

FIG. 22C shows the midline cross-section of a topical device with a support layer made of a tear-resistant woven fabric 320 and pliable polymer 322 at the periphery, an adhesive layer with overhang 324 at the edge, and release liners. An off-center slit 326 is provided between the release liners.

FIG. 22D shows the midline cross-section of a topical device having a support layer made of a tear-resistant woven fabric 332 and a pliable polymer 330 at the central section, an adhesive layer, and a single continuous release liner 334 without a slit.

FIG. 22E shows the midline cross-section of a topical device having a support layer made of a tear-resistant woven fabric 344 near the outer edge, a high-tear-resistant woven fabric 342 next to the outer edge and a pliable polymer 340 at central section; a non-continuous adhesive layer 346; and release liners.

FIG. 22F shows the midline cross-section of a topical device having a support layer made of a high tear-resistant woven fabric 354 near the outer edge, a tear-resistant woven fabric 352 next to the woven fabric 354 and a pliable polymer 350 at central section; an adhesive layer 346; and release liners with multiple slits 356 and 358.

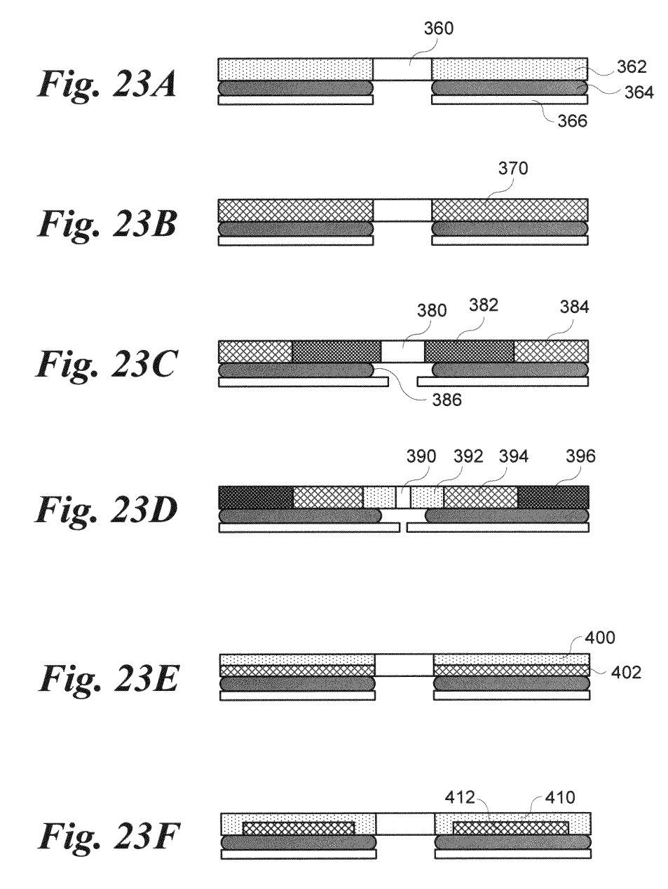

FIG. 23A shows the midline cross-section of a circular topical device having a pliable polymer support layer 362, an adhesive layer 364, and a release liner 366. The device has a central opening 360 that may be positioned at or near the anus or the anal aperture.

FIG. 23B shows the midline cross-section of a circular topical device having a tear-resistant woven fabric support layer 370, an adhesive layer, a release liner, and a central opening. The central opening may be positioned at or near the anus or the anal aperture.

FIG. 23C shows the midline cross-section of a circular topical device having support layer made of a tear-resistant woven fabric 384 at the outer ring, a high-tear-resistant woven fabric 382 at the inner ring and a small central opening 380. The device further has an adhesive layer with a recess 386 near the central hole and a release liner.

FIG. 23D shows the midline cross-section of a circular topical device having support layer made of a high tear-resistant woven fabric 396 at the outer ring, a tear-resistant woven fabric 394 at the first inner ring, a pliable polymer 392 at the second inner ring and a very small central opening 390. The device further has an adhesive layer with a recess near the central hole and a release liner.

FIG. 23E shows the midline cross-section of a circular topical device having a support layer made of a laminate of a pliable polymer 400 at the outer surface and a tear-resistant woven fabric 402 at the next layer. The device further has an adhesive layer, a release liner and a central opening.

FIG. 23F shows the midline cross-section of a circular topical device having a support layer made of a composite of a pliable polymer 410 and a tear-resistant woven fabric 412, wherein the woven fabric 412 is at least partially embedded in the pliable polymer 410. The device further has an adhesive layer, a release liner and a central opening.

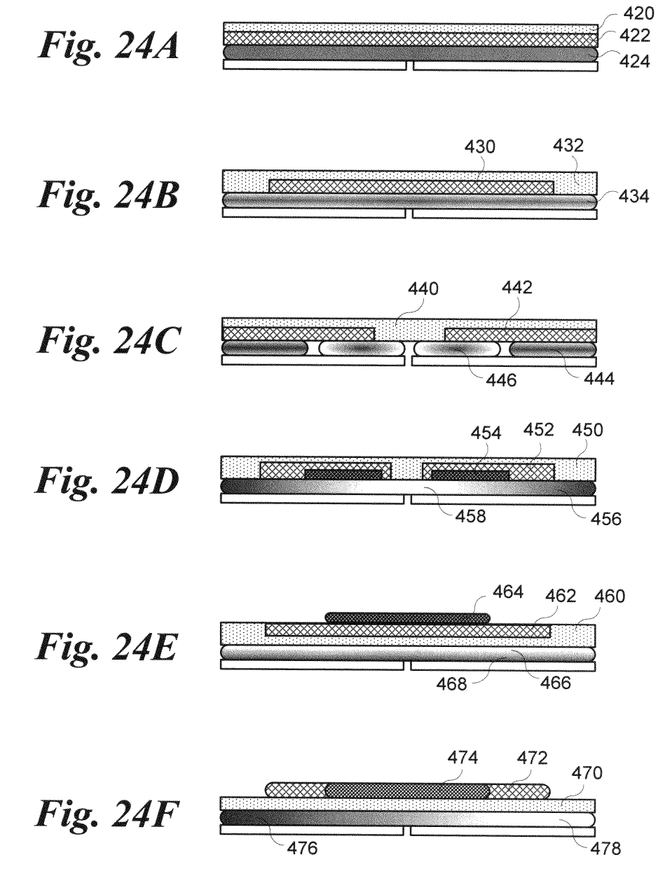

FIG. 24A shows the midline cross-section of a topical device having a support layer made of a laminate of a pliable polymer 420 at the outer surface and a tear-resistant woven fabric 422 at the next layer. The device further has an adhesive layer 424 and release liners. The adhesive layer 424 or the support layer 420 and 422 may contain one or more additives selected from the group comprising a solvent, a chemical agent, a pharmaceutical agent, a biological agent, or combinations thereof. In this example, it is preferred that an analgesics is uniformly incorporated into the adhesive layer 424. The anagesics may be selected from the list comprising lignocaine, benzocaine, lidocaine, benzyl nicotinate, methyl salicylate, diethylamine salicylate, trolamine salicylate, salicylamide, camphor, menthol, and dimethyl sulfoxide.

FIG. 24B shows the midline cross-section of a topical device having a support layer made of a composite with a tear-resistant woven fabric 430 embedded within and coated with a pliable polymer 432 at the outer surface. The device further has an adhesive layer 434 and release liners. The adhesive layer 434 or the support layer 420 and 422 may contain one or more of anagesics selected from the list of lignocaine, benzocaine, lidocaine, benzyl nicotinate, methyl salicylate, diethylamine salicylate, trolamine salicylate, salicylamide, camphor, menthol, and dimethyl sulfoxide. In this example, the analgesics is non-uniformly incorporated throughout the thickness of the adhesive layer 434.

FIG. 24C shows the midline cross-section of a topical device having a support layer made of a composite with a tear-resistant woven fabric 442 embedded within and coated with a pliable polymer 440 at the outer surface. The device further has an adhesive layer 444 and 446 and release liners. The adhesive layer 444 may contain one or more additives selected from the group comprising a solvent, a chemical agent, a pharmaceutical agent, a biological agent, or combinations thereof. The adhesive layer 446 may contain one or more additives selected from the group comprising a solvent, a chemical agent, a pharmaceutical agent, a biological agent, or combinations thereof, which are different than those of the adhesive layer 444.

FIG. 24D shows the midline cross-section of a topical device having a support layer made of a composite with a high tear resistant woven fabric 454 and a tear-resistant woven fabric 452 embedded within and coated with a pliable polymer 450 at the outer surface. The device further has an adhesive layer and release liners. The adhesive layer near the periphery 456 may contain higher concentration of one or more additives selected from the group comprising a solvent, a chemical agent, a pharmaceutical agent, a biological agent, or combinations thereof, than that of the adhesive layer near the center 458. FIG. 24E shows the midline cross-section of a topical device having a support layer made of a composite with a tear-resistant woven fabric 462 embedded within a pliable polymer 460. A high tear-resistant woven fabric or a rigid plastic sheet 464 may be laminated onto the outer surface of the support layer near the central region for further reinforcement. The device further has an adhesive layer and release liners. The adhesive layer may contain one or more of anagesics selected from the list of lignocaine, benzocaine, lidocaine, benzyl nicotinate, methyl salicylate, diethylamine salicylate, trolamine salicylate, salicylamide, camphor, menthol, and dimethyl sulfoxide. In this example, the analgesics is incorporated with a higher concentration near the tissue-contacted surface 468 than near the support layer 466.

FIG. 24F shows the midline cross-section of a topical device having a support layer made of a laminate of a pliable polymer 470 and a composite of a tear-resistant woven fabric 472 with a high tear-resistant woven fabric or a rigid plastic sheet 474. The device further has an adhesive layer and release liners. The adhesive layer may contain one or more of anagesics selected from the list of lignocaine, benzocaine, lidocaine, benzyl nicotinate, methyl salicylate, diethylamine salicylate, trolamine salicylate, salicylamide, camphor, menthol, and dimethyl sulfoxide. In this example, the analgesics is incorporated with a higher concentration at a first end 476 of the adhesive layer than at a second end 478 of the adhesive layer.

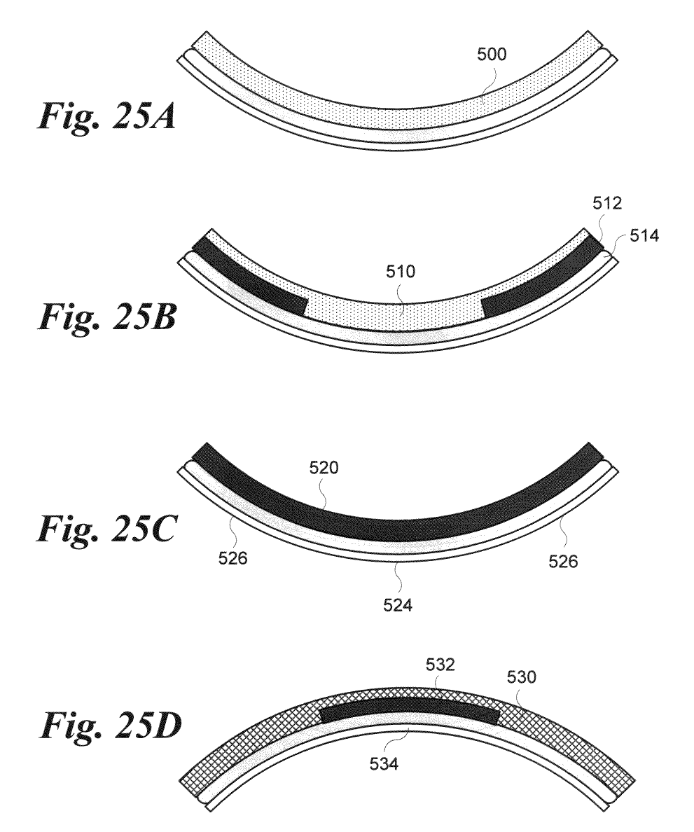

FIG. 25A shows an example of a cross-section of a topical device having a pliable polymer support layer 500, an adhesive layer and a release liner. The device has a contour surface that is concave on the support layer 500 side as shown in FIG. 25A. Alternately, the device may have a contour surface that is convex on the support layer side. Since the device is flexible, it may be converted from concave to convex or vice versa.

FIG. 25B shows an example of a cross-section of a topical device having support layer made of a laminate of a flexible polymer 510 and a rigid polymer layer 512. The device further has an adhesive layer 514 and a release liner. The adhesive layer 514 is in contact with both the pliable polymer layer 510 and the rigid polymer layer 512. The device has a contour surface that is concave on the support layer side as shown in FIG. 25B. Alternately, the device may have a contour surface that is convex on the support layer side. The device shown in FIG. 25B is relatively non-planar.

FIG. 25C shows an example of a cross-section of a topical device having a rigid or substantially rigid polymer support layer 520, an adhesive layer and a release liner. The device has a contour surface that is concave on the support layer 520 side as shown in FIG. 25C. Alternately, the device may have a contour surface that is convex on the support layer side. The radius of curvature may change from one location to another along the device length, width or diameter. For example, the radius of curvature may be substantially smaller for the midline or central section 524 of the device compared to the lateral or the peripheral sections 526. In this case, the cross-section shown in FIG. 25C may look more pointed at or near the midline or central section 524. In another example, the radius of curvature may be substantially larger for the midline or central section 524 of the device compared to the lateral or the peripheral sections 526. In this case, the cross-section shown in FIG. 25C may have a less contour surface at the midline or central section 524. The non-planar shape of the device may help resist push-out forces acting substantially perpendicular to the midline or central section 524. Examples of a topical device with the cross-section shown in FIG. 25C include a rectangular sheet, a square sheet, a round sheet, a triangular sheet, a trapezoidal sheet, or sheet with an irregular shape that is curved, contoured, bent or partially folded.

FIG. 25D shows an example of a cross-section of a topical device having a support layer made of a laminate of a tear-resistant woven or knitted fabric 530 and a rigid or substantially rigid polymer layer 532, wherein the polymer layer 532 is located at or near the midline or central section 534 of the device. The device further has an adhesive layer and a release liner. The adhesive layer is in contact with both the woven fabric layer 530 and the polymer layer 532. The device has a contour surface that is convex on the support layer side as shown in FIG. 25D. Alternately, the device may have a contour surface that is concave on the support layer side. Since the device is at least partially flexible or conformable, it may be converted from concave to convex or vice versa.

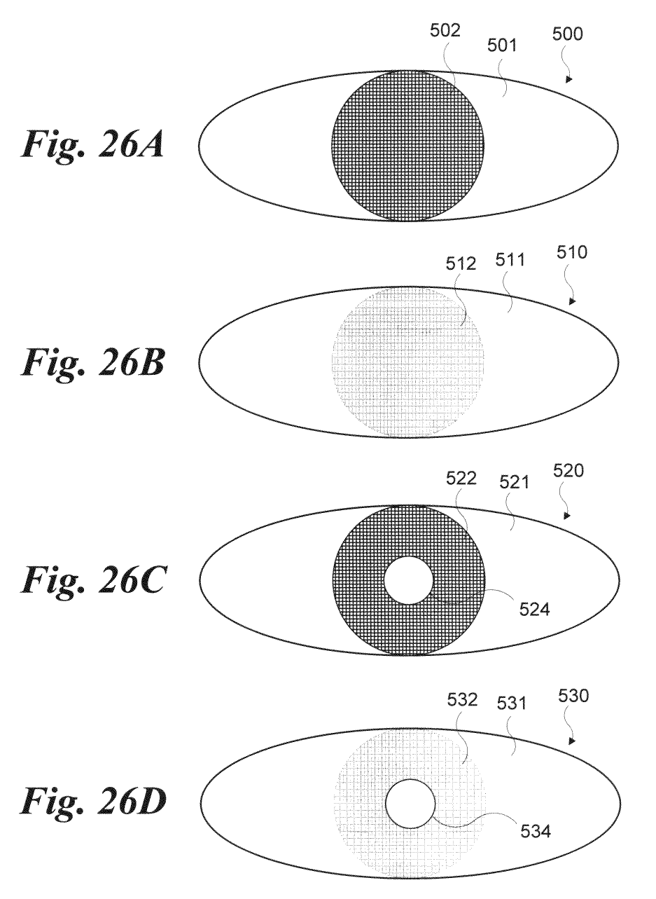

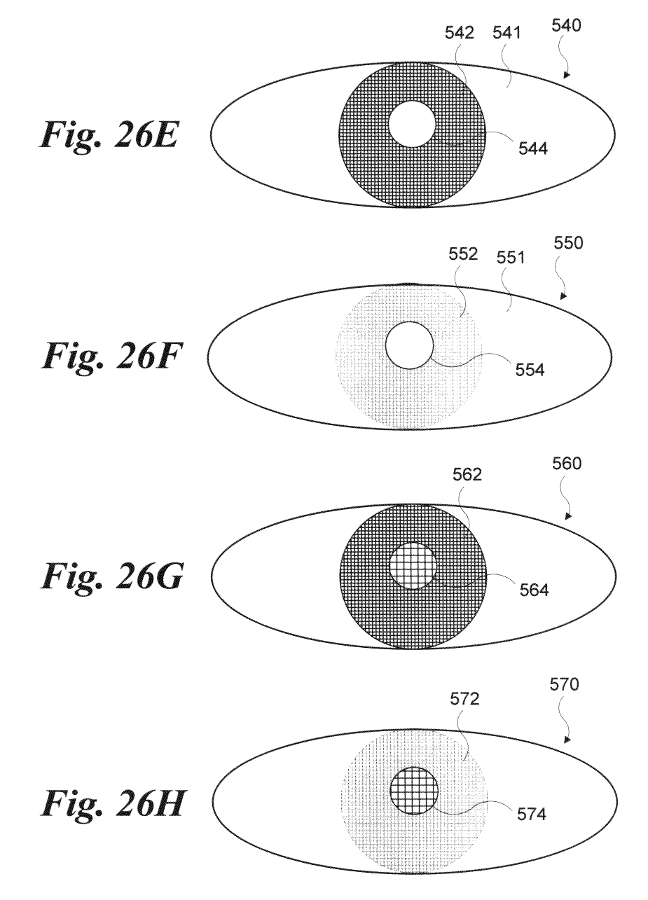

FIG. 26A shows the top view of a substantially planar topical device 500 that is made of an oval-shaped (or a rectangular-shape with rounded corners) adhesive patch 501 reinforced with a circular patch 502. The oval adhesive patch 501 is tear resistant and may be made of a stretchable, semi-stretchable, or non-stretchable material such as a polymer film, a non-woven mesh, a woven mesh, a knitted mesh, a mesh reinforced or embedded in a polymer film, or other tear-resistant materials described above. The circular reinforcing patch 502 preferably has equal or greater tear-resistance than the oval adhesive patch 501. It may be made of any tear-resistant materials such as a toughened plastic film, a non-woven mesh, a woven mesh, a knitted mesh, a mesh reinforced or embedded in a polymer film, or other tear-resistant materials described above. One example of the topical device 500 is a pliable, semi-stretchable, adhesive patch made of a Tricot fabric adhesive tape produced by 3M Corporation (Minnesota) or other suppliers. The oval adhesive patch 501 can be made of a Tricot fabric adhesive tape while the circular reinforcing patch 502 can be made of a Tricot adhesive tape, a polyester film, a polyethylene film, a woven fabric adhesive tape, a non-woven fabric adhesive tape or another type of material with similar or better tear-resistance compared to the Tricot fabric tape. The circular reinforcing patch 502 is preferred to be adhesively fixed to the oval adhesive patch 501. It may be preferred that the circular reinforcement patch is made of a material or a weave/knit pattern that offers both higher stretching-resistance and higher tear resistance. The reinforcing patch or layer may also have a non-circular shape such as a rectangular, square, oval, oblong, trapezoidal, triangular, or diamond shape.

The topical device 500 may be positioned and adhesively secured to the perineal or perianal area such that the anus approximates the center of the device. The topical device 500 with substantial tear-resistance may reduce, limit or prevent laceration from approaching or reaching the anus. The topical device 500 with the reinforcing circular patch 502 may help divert, deflect or redirect a medial laceration toward a mediolateral direction, and thus, may reduce the risk of damage to the anal sphincter and the rectal mucosa. The device may also help reduce, limit or prevent other tissue damages by limiting excessive stretching. Although the topical device 500 is tear-resistant, it is preferred to be not cut-resistance. It may be necessary for the topical device to be easily cut with a pair of episiotomy scissors or a surgical scalpel in case the obstetricians or the midwives would like to perform an episiotomy or to extend an existing episiotomy in the perineal area covered by the topical device 500. Thus, the topical device 500 is preferred to be tear-resistant but not cut-resistance in order to prevent or reduce inadvertent tissue damages while not interfering with an episiotomy or the child-birth delivery process. It may be preferred that the circular reinforcing patch 502 is more cut-resistant than the oval adhesive patch 501 in order to provide more protection to the perianal area when an episiotomy incision or extension of an episiotomy incision is performed in the presence of the topical device 500. The oval adhesive patch 501 and the circular reinforcing patch 502 may be designed to have different levels of cut-resistance so that excessive or extended episiotomy may be preferably forced to go around the circular reinforcing patch 502 in order to reduce the risk of damage to the anal sphincter and the rectal mucosa.

FIG. 26B shows the top view of a topical device 510 similar to the one shown in FIG. 26A except the circular reinforcing patch 512 is located below the oval adhesive patch 511. Attachment of the circular reinforcing patch 512 to the oval adhesive patch 511 may be achieved via a variety of means including lamination, bonding, fusion, embedding, or combinations thereof. As the device 510 is adhesively bonded or secured to the perineal and perianal areas, it is preferred that the circular reinforcing patch 512 is at least partially aligned with the midline connecting the vagina and the anus in order to reduce, limit or prevent a laceration or an episiotomy-induced tear from approaching or reaching the anus.

FIG. 26C shows the top view of a topical device 520 similar to the one shown in FIG. 26A except the circular reinforcing patch 522 and the oval adhesive patch 521 have a concentric opening 524 at the center. Such an opening is intended to avoid or reduce possible pressure built up by gas, fluid, fecal matter, or extruding anal tissues that may adversely affect the attachment, affixing, bonding, or fixation of the topical device. The topical device may be positioned and adhesively secured to the perineal area such that the anus approximates the central opening of the device. The tear-resistant nature of the topical device 520 may reduce, limit or prevent a laceration or an episiotomy-induced tear from approaching or reaching the anus.

FIG. 26D shows the top view of a topical device 530 similar to the one shown in FIG. 26C except the circular reinforcing patch 532 is located below the oval adhesive patch 531. Reinforcement may be achieved via a variety of means including lamination, bonding, fusion, embedding, weaving, knitting, or combinations thereof. As the device is adhesively bonded or secured to the perineal area, it is preferred that the circular reinforcing patch 532 is at least partially aligned with the midline connecting the vagina and the anus in order to reduce, limit or prevent a laceration or an episiotomy-induced tear from approaching or reaching the anus.

FIG. 26E shows the top view of a topical device 540 similar to the one shown in FIG. 26C except the circular reinforcing patch 542 and the oval adhesive patch 541 have a non-concentric opening 544 near the center. Such an opening is intended to avoid or reduce possible pressure built up by gas, fluid, fecal matter, or extruding anal tissues that may adversely affect the attaching, affixing, bonding, or fixation of the topical device. The topical device may be positioned and adhesively secured to the perineal area such that the anus approximates the central opening of the device. The tear-resistant nature of the topical device 540 may reduce, limit or prevent laceration from approaching or reaching the anus.

FIG. 26F shows the top view of a topical device 550 similar to the one shown in FIG. 26E except the circular reinforcing patch 552 is located below the oval adhesive patch 551. FIG. 26G and FIG. 26H show the top views of similar devices 560 and 570 as shown in FIG. 26E and FIG. 26F, respectively, except the topical devices 560 and 570 have an opening 564 and 574 covered with a porous mesh in order to avoid or reduce possible pressure built up by gas, fluid, or fecal matter, while reducing extruding anal tissues that may adversely lead to tissue damages or pain.

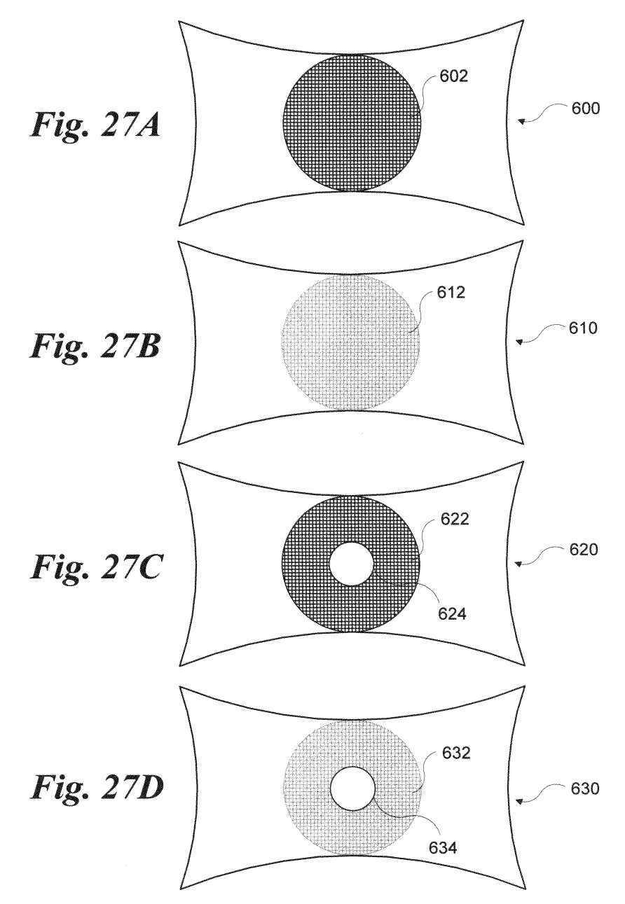

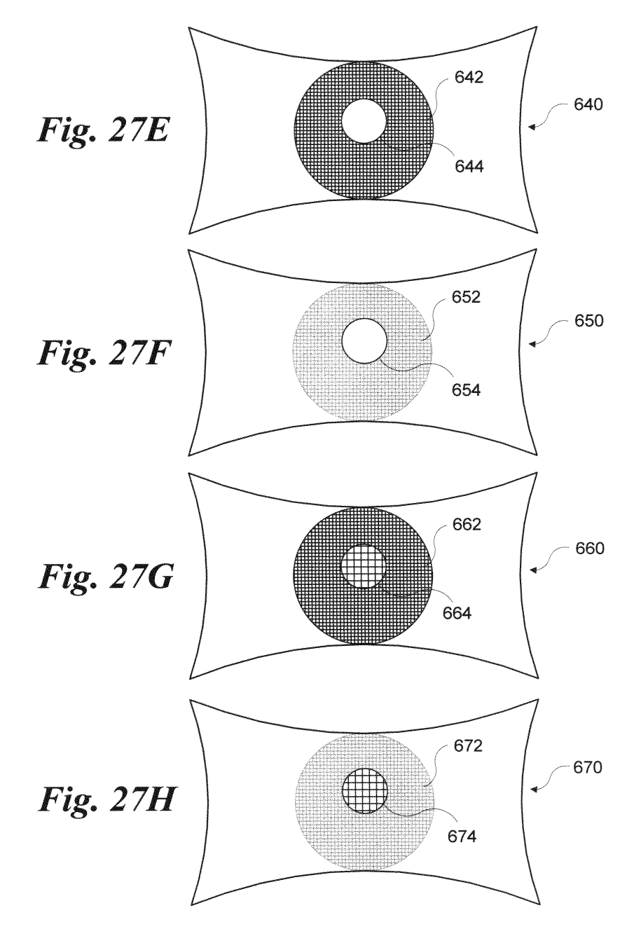

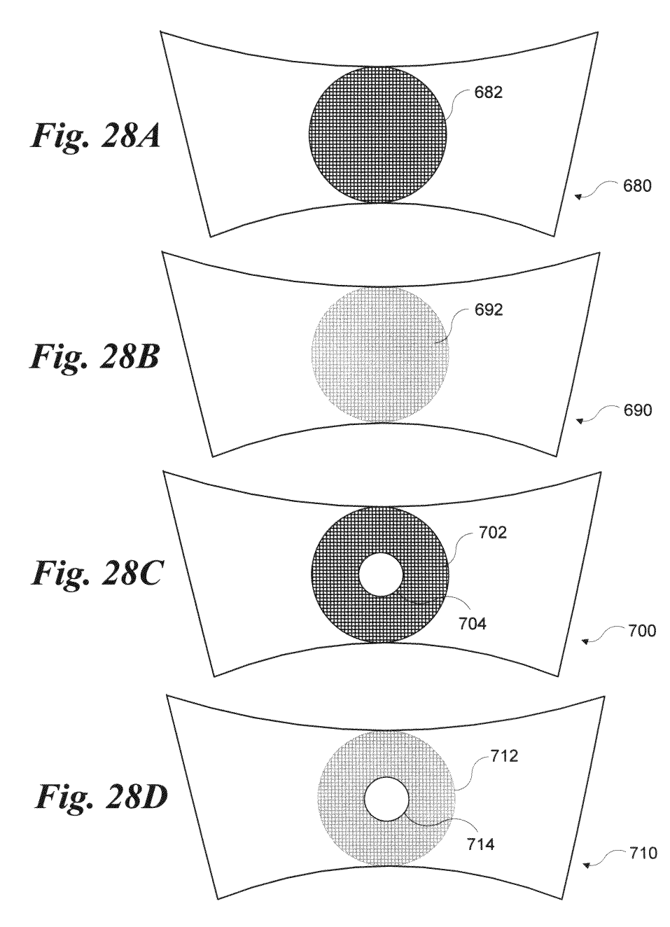

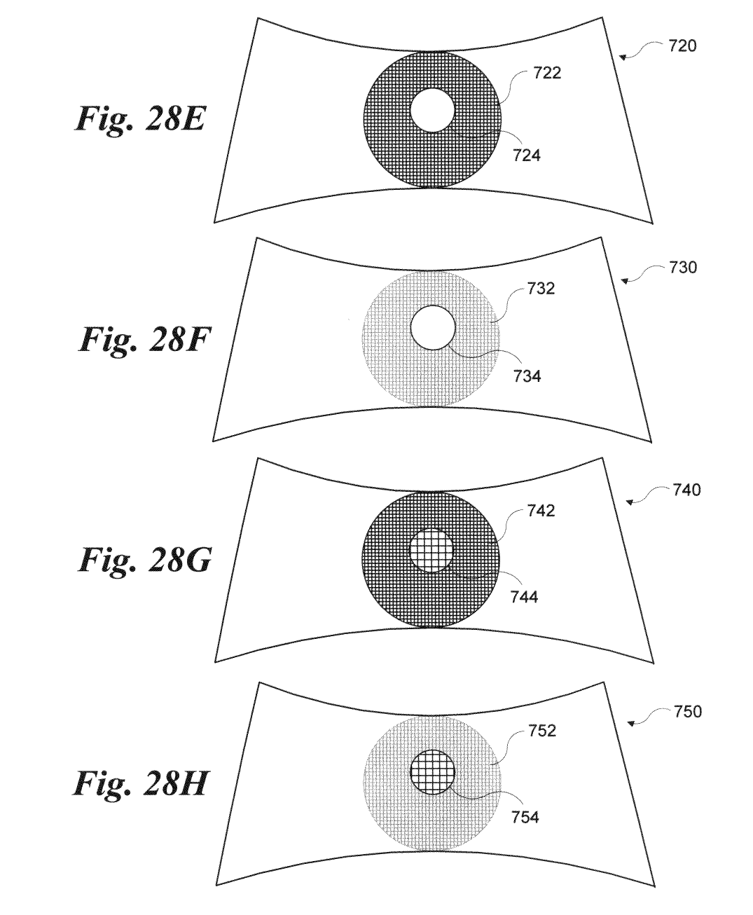

FIGS. 27A-27H show topical devices similar to the ones shown in FIGS. 26A-26F except, the oval adhesive patch is replaced with a relatively rectangular with four concave sides or edges. FIGS. 28A-28H show topical devices similar to the ones shown in FIGS. 26A-26F except, the oval adhesive patch is replaced with an elongated or trapezoidal shape with concave top and bottom (longer) sides or edges. FIGS. 26-28 show examples of some useful shapes or configurations of the topical device. Other shapes or configurations of the topical devices with concave sides or edges are also contemplated and not limited by these examples. Concave edges, especially for the vagina-facing edge of the patch, may provide the topical patch with enhanced resistance to stress, strain, tear, delamination, etc. It may be preferred that the topical devices have at least one concave edge. It may also be preferred that such topical devices are positioned such that the at least one concave edge faces the vagina, the laceration, the episiotomy incision, or the episiotomy-induced tear. In applications outside of perineal support or protection, it may be preferred that the concave edge is placed opposite to the tissue incision, the tissue tear, the tissue defect, or the compromised tissue. The level or degree of concavity of the edges can vary from slightly concave to highly concave. In the case of a round or partially round concave edge, the approximate radius of curvature of the concave edge may be in the range of 0.5 cm to 200 cm, preferably 1 cm to 50 cm, more preferably 2 cm to 20 cm. It may be preferred that the concave edges and their vicinities have different material compositions, material properties, weave patterns, reinforcements, or adhesive properties compared the other areas of the topical device for enhanced performance.

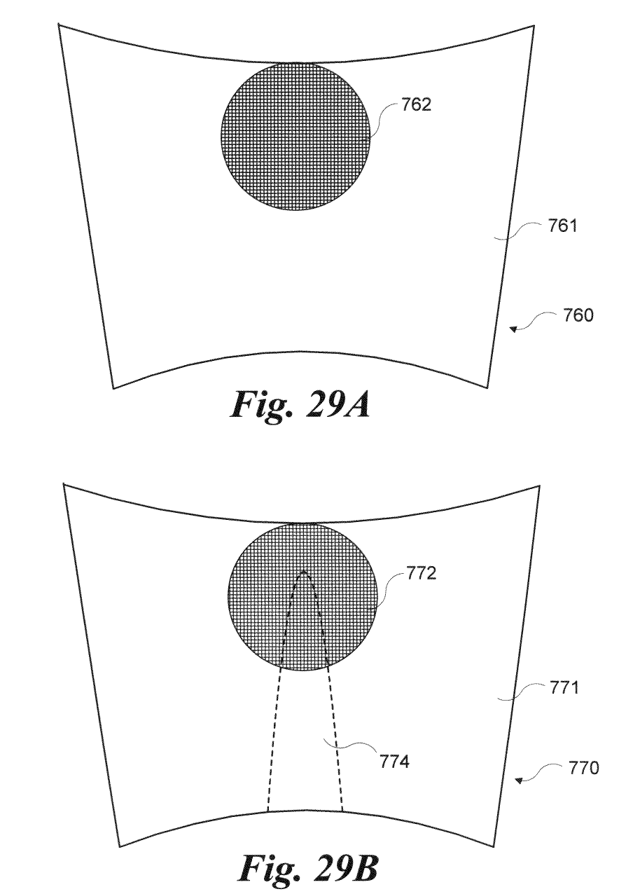

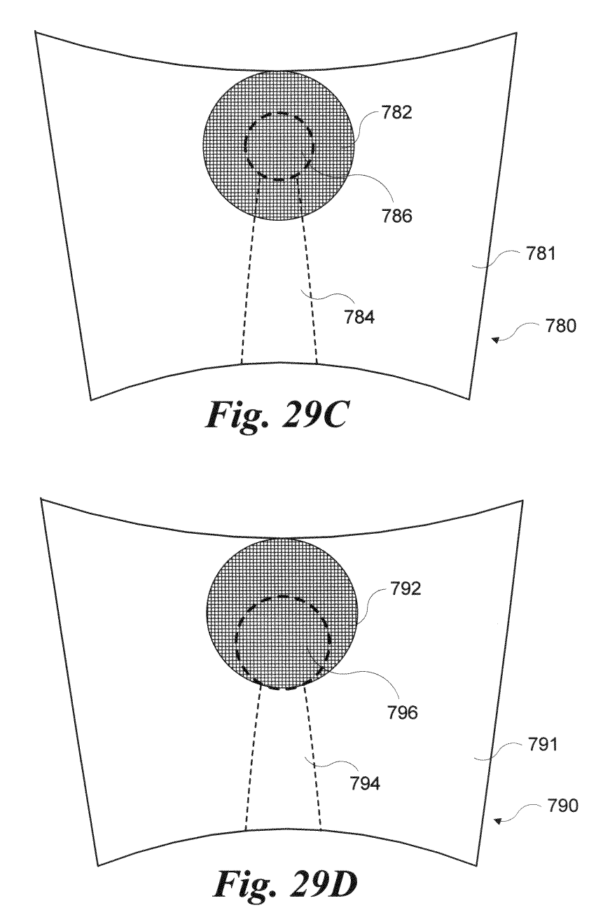

FIG. 29A shows a topical device 760 similar to the one shown in FIG. 28A except the adhesive patch 761 is enlarged along the axis of symmetry to become a relatively square for increased adhesive contact and fixation to patient skin. A circular reinforcement patch 762 is affixed adjacent to a concave edge of the adhesive patch 761. The reinforcement patch 762 may have any shapes including but not limited to a rectangular, triangular, oval, oblong, or any elongated shape disposed along a concave edge of the adhesive patch 761. FIG. 29B shows a topical device 770 similar to the one shown in FIG. 29A except the adhesive patch 771 has an adhesive-free channel 774 on the skin-contacting side along the mid-line to allow drainage of discharged gas, fluid, or fecal matter away from the vagina and the exiting baby. The adhesive-free channel may be an elongated channel running substantially along the midline from the anus toward the coccyx. This may help improve sanitation, reduce contamination, and decrease the potential for infection of the patient and/or the baby.

FIGS. 29C and 29D show two embodiments of the topical device of the present invention, wherein a first portion of the adhesive layer, which is opposite to, and has an area less than an area of, the reinforcement layer, and a second portion of the adhesive layer, which is contiguous with the first portion and extends away from the reinforcement layer, are free of adhesive. FIG. 29C shows a topical device 780 similar to the one shown in FIG. 29B except the adhesive patch 781 has a concentric adhesive-free area 786 below the reinforcing patch 782 and a connecting non-adhesive channel 784 on the skin-contacting side along the mid-line to allow drainage of patient's anal discharge or secretion of gas, fluid, or fecal matter away from the vagina and the exiting baby.

FIG. 29D shows a topical device 790 similar to the one shown in FIG. 29C except the adhesive patch 791 has a non-concentric adhesive-free area 796 below the reinforcing patch 792 and a connecting non-adhesive channel 796 on the skin-contacting side along the mid-line to allow drainage of patient's anal discharge or secretion of gas, fluid, or fecal matter away from the vagina and the exiting baby.

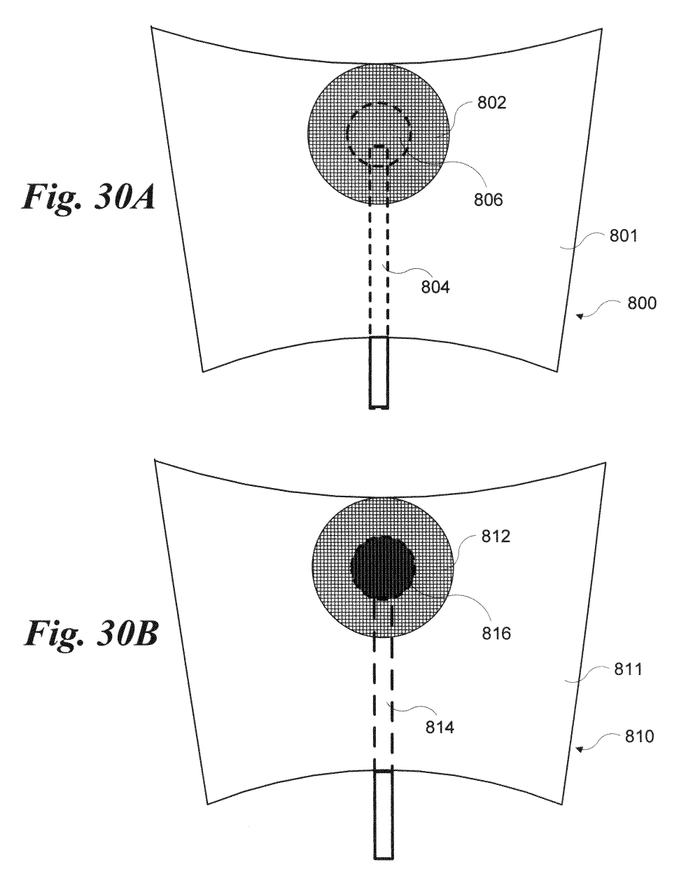

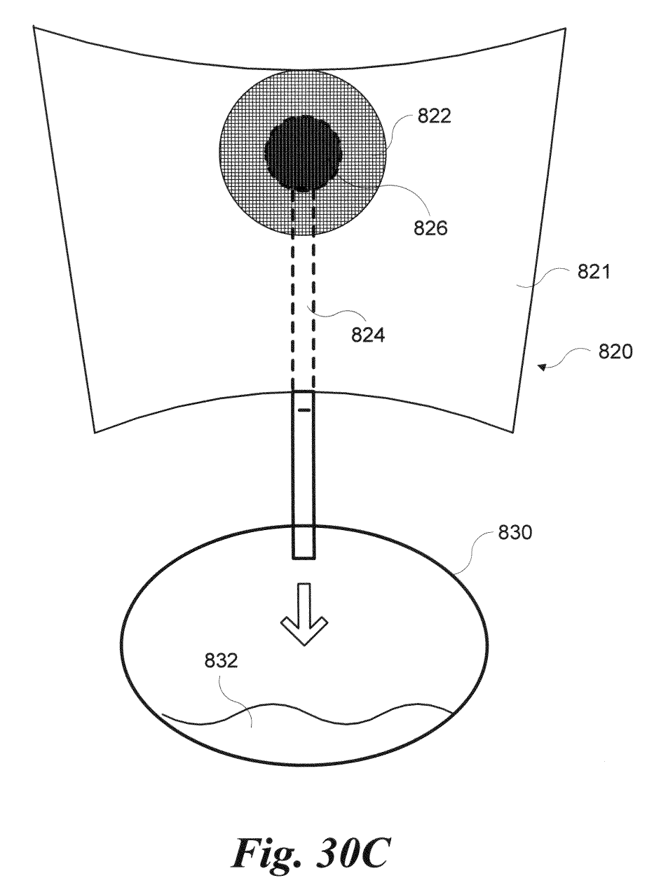

FIG. 30A shows a topical device 800 similar to the one shown in FIG. 29C except the adhesive-free channel is replaced with a tubing 804 for possibly more effective drainage of patient's anal discharge or secretion of gas, fluid, or fecal matter away from the vagina and the exiting baby. The tubing 804 may be flexible, semi-flexible or rigid. The tubing can be made of an elastomer, a rubber, a plastic or a metal. The tubing 804 may have a cross-section of any size and shape including round, rectangle, triangle, irregular shapes, etc. Examples of tubing materials include silicone, polyurethane, thermoplastic elastomers, nitrile rubbers, PVC, nylon, polyethylene, polypropylene, stainless steel, etc.

FIG. 30B shows a topical device 810 similar to the one shown in FIG. 30A except the adhesive patch 811 has an adhesive-free cap 816 located below the reinforcing patch 812 that is connected with the tubing 814 to allow drainage of patient's discharge or secretion of gas, fluid, or fecal matter away from the vagina and the exiting baby. The cap 816 is preferred to be located approximately above the anus. The cap 816 may be planar or non-planar relative to the topical device prior to application onto a patient. The cap 816 may be concave or convex relative to the patient-contacting side of the topical device. The cap 816 may be flexible, semi-flexible or rigid. The cap 816 can be made of an elastomer, a rubber, a plastic or a metal. The cap 816 may function as a drainage reservoir cap with increased space between the anus and the device for temporary collection of anal secretion prior to discharging through the connected tubing or an adhesive-free channel. The cap 816 may be in configuration of a flexible pouch, an expandable membrane, or a dome-shaped object. Examples of materials for the drainage reservoir cap include silicone, polyurethane, thermoplastic elastomers, nitrile rubbers, PVC, nylon, polyethylene, polypropylene, polyester, acrylics, polycarbonate, titanium, stainless steel, etc. Examples of the reservoir 816 include a foldable/collapsible pouch, an expandable/inflatable pouch, a flat and stretchable film or membrane, a flexible dome-shaped cap, a semi-rigid dome-shaped cap, a rigid dome-shaped cap, a hemi-sphere shell, a non-rounded convex cap, a clam-shell-shaped cap, etc.

FIG. 30C shows a topical device 820 similar to the one shown in FIG. 30B except the tubing 824 may be connected to a drainage container or bag 830 for collecting patient's anal discharge 832. The complete device including the discharge-collecting container is preferably a single-use disposable item. The device may be made modular such that the adhesive patch 820 and the drainage assembly including the reservoir 826, the tubing 824 and the discharge-collecting container 830 can be packaged, delivered or used either together or separately. The bag 830 can be made of an elastomer, a rubber, a plastic or a metal. Example of the materials for the drainage tubing or drainage container include silicone, polyurethane, thermoplastic elastomers, nitrile rubbers, PVC, nylon, polyethylene, polypropylene, polyester, acrylics, polycarbonate, etc.

In addition to the conventional manufacturing methods for adhesive patches, the topical devices shown in FIGS. 29A-D and FIGS. 30A-C may be molded into, at least, near final shapes using a polymeric material such as silicone, polyurethane, thermoplastic elastomers, nitrile rubbers, PVC, nylon, polyethylene, polypropylene, polyester, acrylics, polycarbonate, etc. before laminating with an adhesive layer. The molding methods include injection molding, compression molding, cast molding, transfer molding, insert molding, over molding, extrusion molding, etc. Molding is particularly useful when the topical device is a three-dimensional object such as the topical device shown in FIG. 30B with a dome-shaped cap 816 and a tubing 814. It may be easier to have these components molded separately and subsequently assembled. The dome-shaped cap 816 in FIG. 30B may have a flat peripheral rim that is, at least, partially overlapped with the ring-shaped reinforcing patch 812. In this case, the reinforcing patch 812 may also work as an adhesive patch to secure and seal the cap 816 to the adhesive patch 811.

The sizes, shapes, configurations and materials of the components shown in FIG. 26 through FIG. 30 are only examples of the topical device. Modifications or changes of any aspects can be made without departing from the invention. For instant, the dome-shaped cap 816 in FIG. 30B may have an oval or triangular shape instead of a round shape. The reinforcing patch 812 may have an oval or rectangular shape instead of a round shape. The tubing 814 may be changed into an open channel with two sidewalls along the midline. The adhesive patch 810 may be made of a thicker adhesive-back sheet of polyurethane instead of a woven adhesive tape.

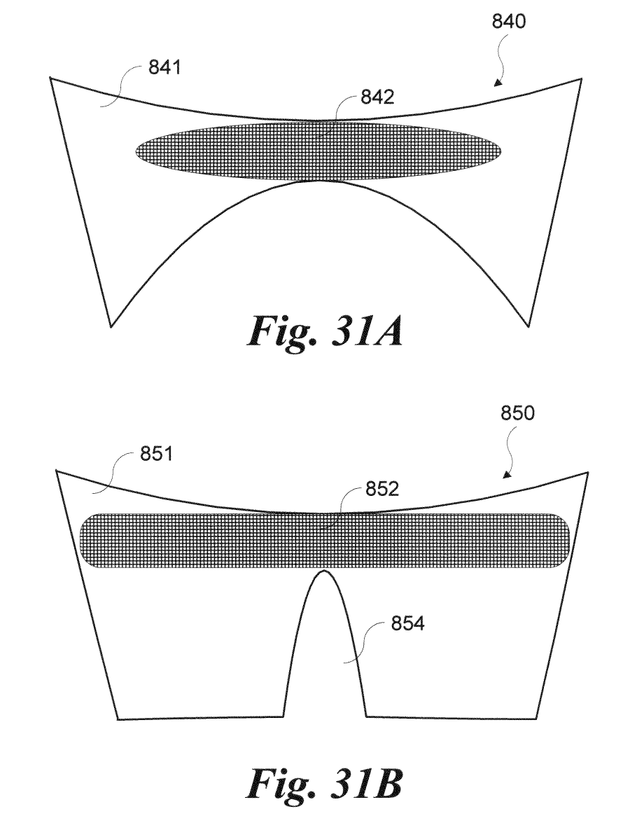

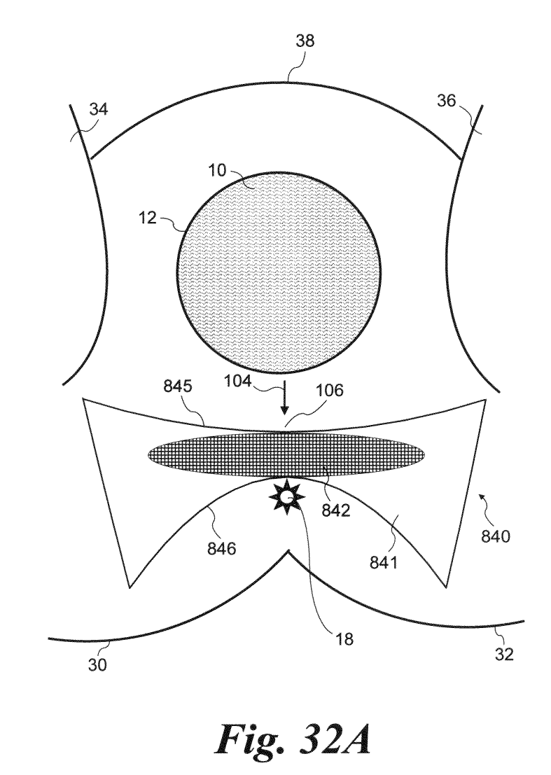

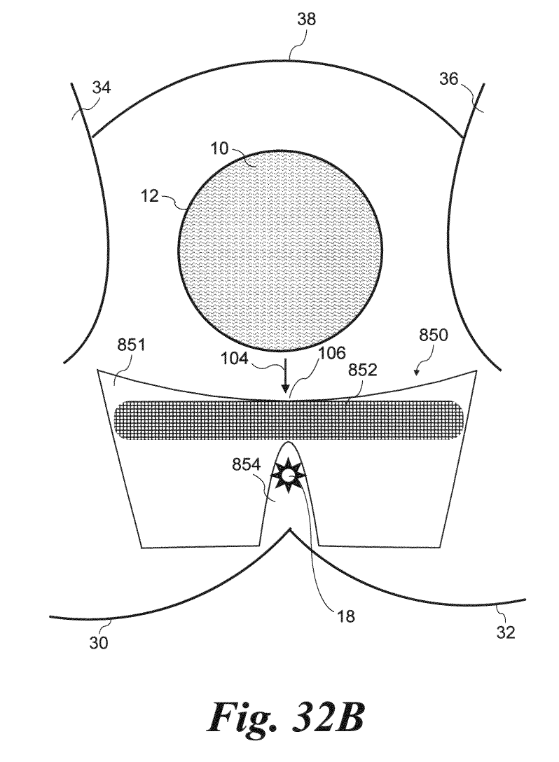

FIG. 31A shows another embodiment of the topical device of the present invention. The device 840 has an adhesive-backed film or patch 841 with extended corners for increased fixation to the patient skin and a reinforcing film or patch 842 with a non-rounded shape to help further resist tear. The topical device 840 is configured to cover primarily an area between the vagina and the anus and areas on both sides of the anus, but does not cover a posterior area opposite to the vagina. In one embodiment, shown in FIG. 31B, topical device 850 has an opening or cut-out extending from an internal region proximate to the reinforced patch 852 to one side of topical device 850. The opening or cut-out is configured to leave the anus open when topical device 850 is attached to the patient. However, for a patient with a short perineal length, the medical staff may choose to place the device such that part or all of the anus is covered by the device. FIG. 32A illustrates the placement and use of the topical device 840 shown in FIG. 31A. The device 840 can be placed approximately between the vagina and the anus with or without covering the anus. The device 840 may be oriented in such a way that the slightly concave edge 845 faces the dilated vagina 12 as shown in FIG. 32A. Alternately, the device 840 may be oriented such that the highly concave edge 846 faces the dilated vagina 12. FIG. 32B illustrates the placement and use of the topical device 850 shown in FIG. 31B. Similarly, the device 850 can be placed between the vagina and the anus with or without covering the anus. As shown in FIG. 32B, one of the concave edges of the topical patch 850 faces the dilated vagina 12. The tear-resistant reinforcement layer 852 is located adjacent to the vagina-facing concave edge. The topical device 840 in FIG. 32A or 850 in FIG. 32B may limit or prevent a laceration 104, which includes a stress- or strain-induced tear, an episiotomy-induced tear, and an episiotomy incision, from advancing toward the anus when it reaches the device near the area 106. The topical device 840 or 850 may divert or deflect a medial laceration 104 toward a lateral direction along its edge. The topical device 840 or 850 may also reduce, limit or prevent a mediolateral tear or incision from progressing when it reaches the concave edge of the patch.

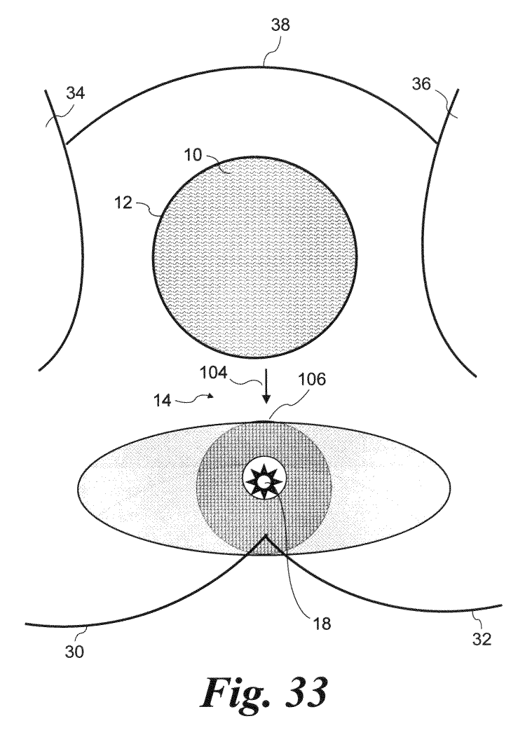

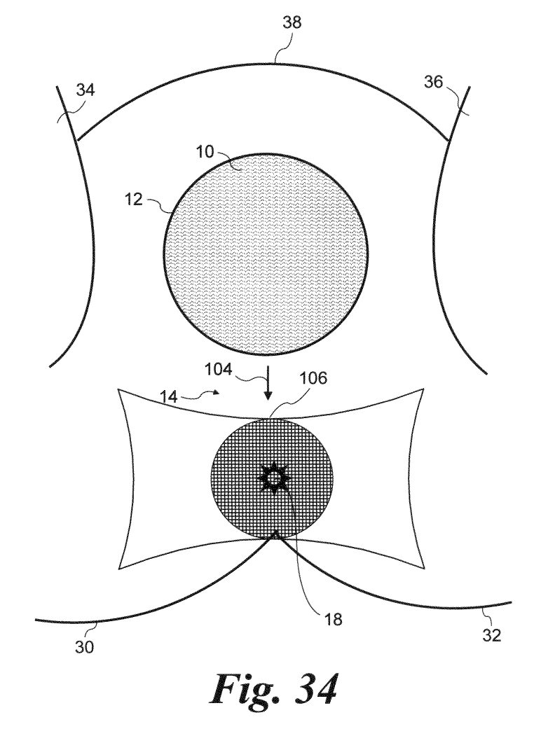

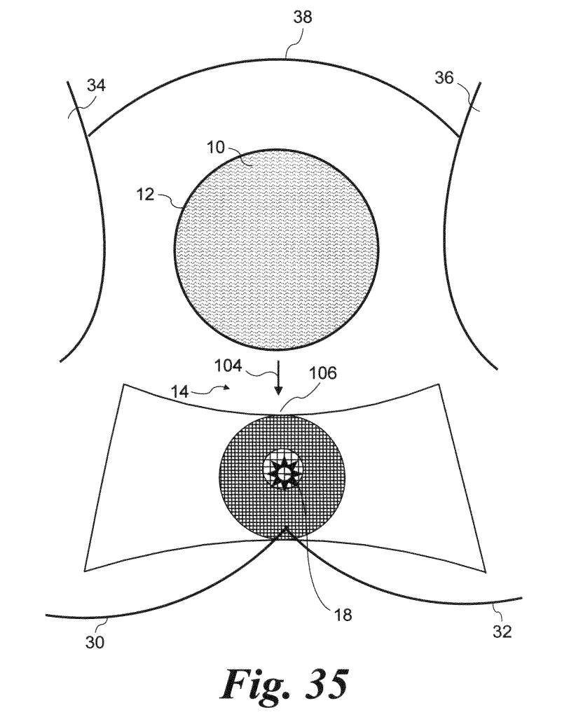

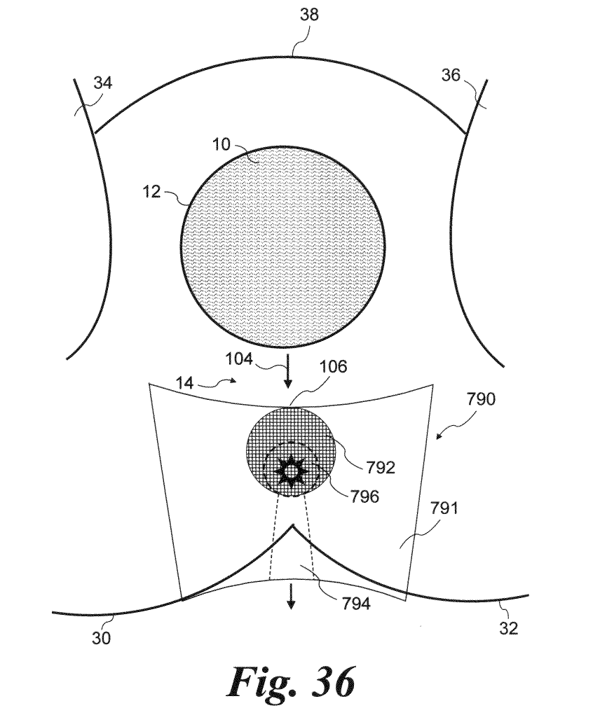

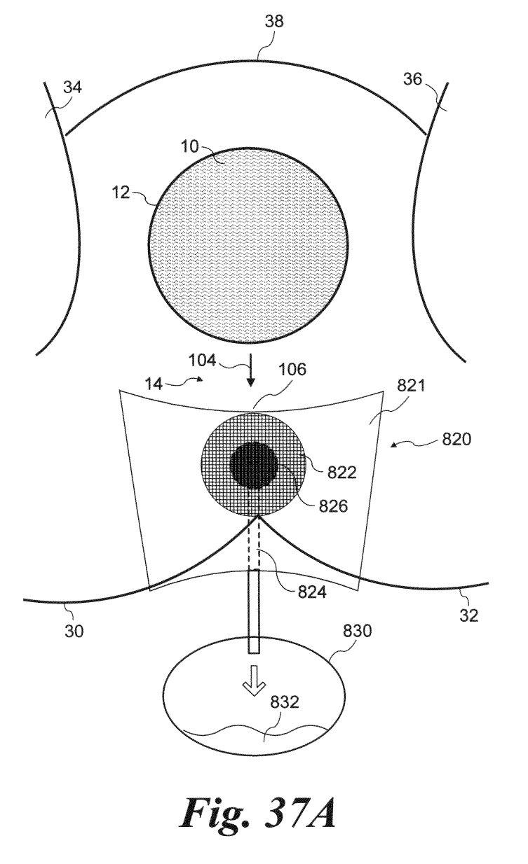

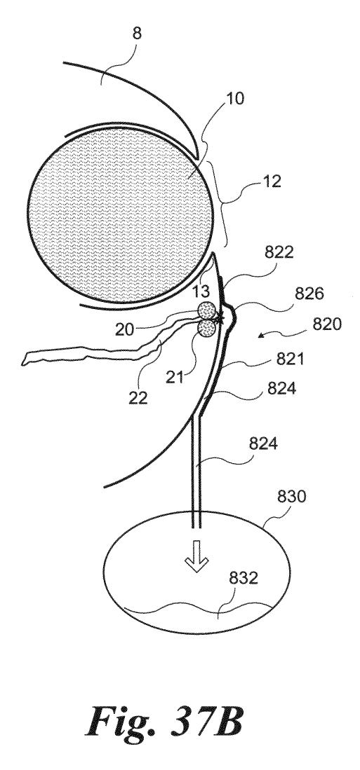

FIGS. 33 through 37 show examples of the placement or application of the topical devices shown in FIG. 26F, FIG. 27A, FIG. 28E, FIG. 29D and FIG. 30C. The topical devices shown in FIGS. 33 through 35 are typically designed (e.g. with opening, vent, porous mesh, etc.) in such a way to reduce potential pressure caused by anal discharge or secretion or budging of perianal tissues. In FIGS. 36, 37A, and 37B, the adhesive-free channel and tubing, respectively, allow drainage of the patient's discharge or secretion of gas, fluid, or fecal matter away from the vagina and the exiting baby.

FIG. 36 shows the anterior view of the topical device 790 shown in FIG. 29D when it is placed or affixed on a patient. The adhesive-free area 796 and the adhesive-free channel 794 allow anal discharge or secretion of gas, fluid, or fecal matter away from the vagina and the exiting baby. FIGS. 37A and 37B show the anterior view and the sagittal midline cross-sectional view, respectively, of the topical device 820 shown in FIG. 30C when it is placed on a patient. FIG. 37A shows the topical device 820 with the tubing 824 connected to the bag 830 for collecting patient's anal discharge or secretion 832. The complete device including the discharge or secretion-collecting bag is preferably a single-use disposable item. The dome-shaped cap 826 is connected with the tubing 824 to allow drainage of patient's discharge or secretion of gas, fluid, or fecal matter away from the vagina and the exiting baby. The dome shape of the cap creates space between the anus and the topical device in order to allow drainage while minimizing or reducing potential pressure built-up by protruding anal tissues.

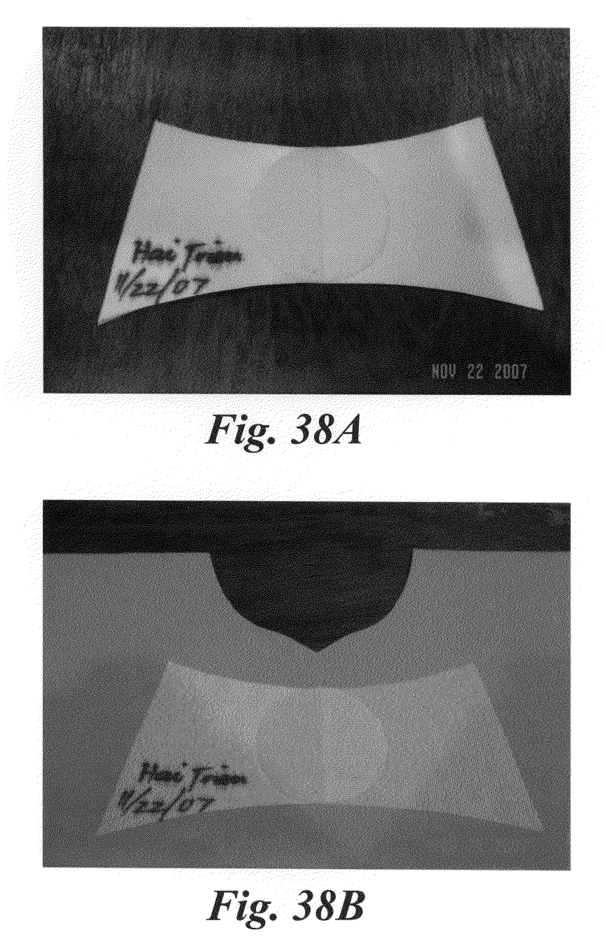

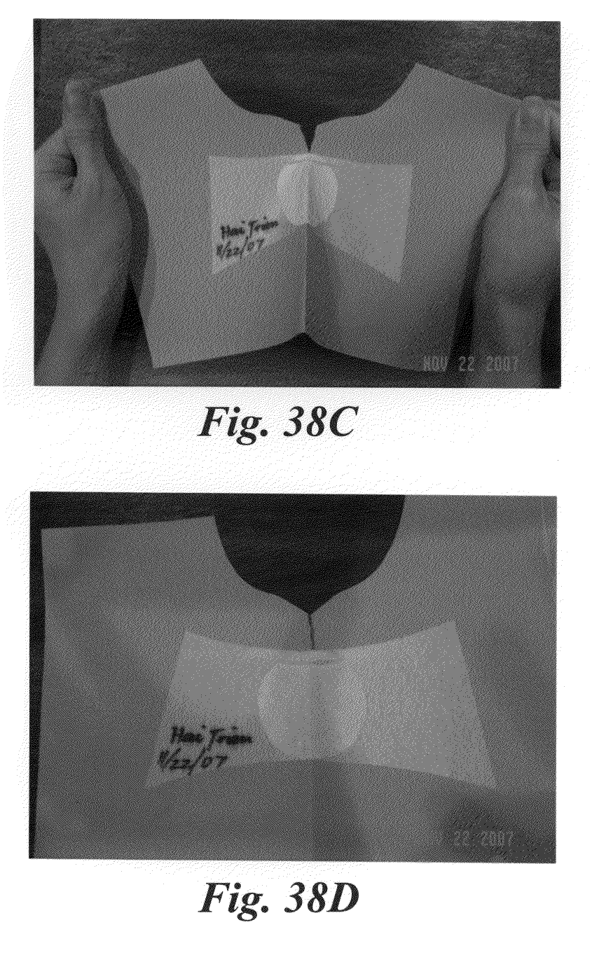

Experiments were carried out to determine whether or not a topical adhesive patch, when made of a tear-resistant material, can help resist or prevent propagation of a tear. In the first experiment, a custom paper model was used to test the ability of the topical adhesive patch to stop or reduce tear propagation. FIG. 38A shows the picture of a prototype of the topical patch illustrated in FIG. 28A. The topical patch was relatively thin, flat, flexible, and conformable. It had slightly concave edges at the top and the bottom sides and a round reinforcing layer laminated on the top surface of the support layer. The support layer had a hypoallergenic or biocompatible adhesive on the bottom surface, which was protected by a release liner. Both the support layer and the round reinforcing layer were made of the same woven fabric tape. FIG. 38B shows the custom paper model of a dilated vagina with a midline tear initiation point (emulating the effect of a highly stressed tissues or an episiotomy cut) and the topical patch affixed onto the perineal area closer to the anus and covering the anus. The topical patch was intentionally not placed adjacent to the edge of the dilated vagina in order to allow adequate vaginal dilation in actual childbirth delivery. It should also be noted that the woven fabric used in this experiment is more tear-resistant in one direction than the other, which is about 90 degrees apart. The topical patch was made with the appropriate orientation of the woven fabric in order to maximize its tear resistance. FIG. 38C shows that a medial or midline tear propagated from the vagina toward the topical patch and the underlying anus. The tear propagating medially was apparently resisted and stopped when it reached the reinforced area of the patch. The concave edge of the patch facing the vagina appeared to have experienced significant tensile force caused by the approaching tear as can be seen in both FIGS. 38C and 38D.

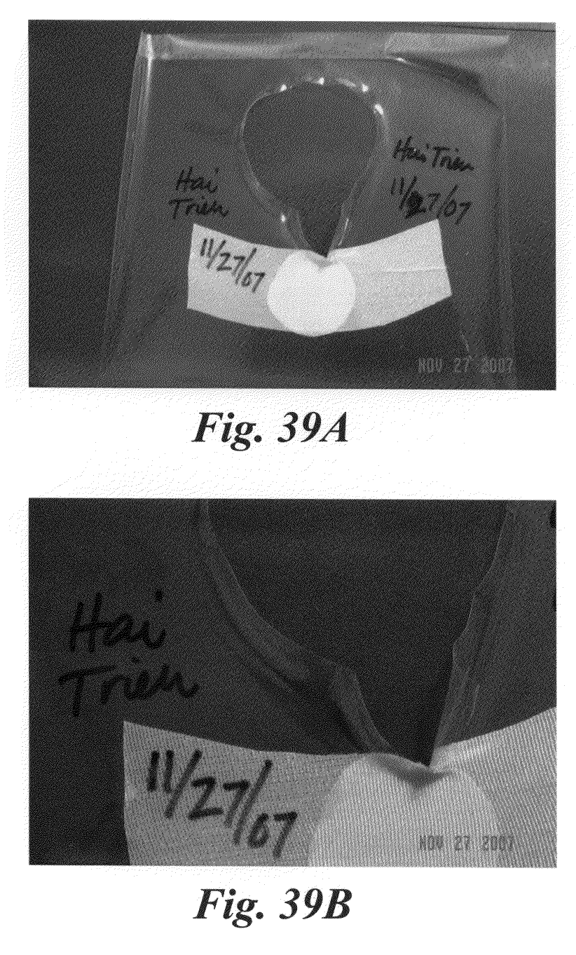

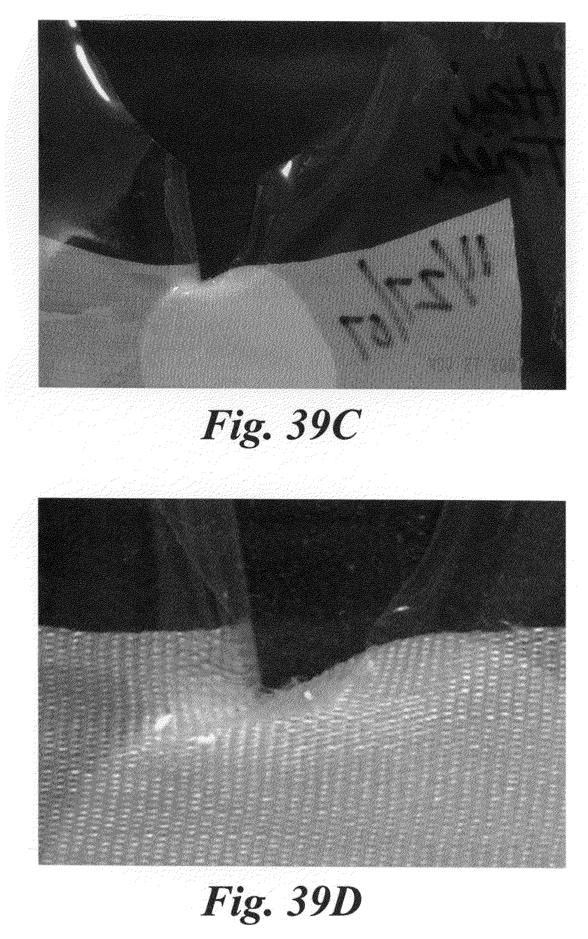

In the second experiment, a custom plastic film model was used to test the ability of a topical adhesive patch to stop or reduce tear propagation through a more ductile material. The vaginal dilation was simulated by stretching the hole in the plastic film in a radial direction in order to cause a tear to propagate toward the topical patch. FIGS. 39A through 39D show the pictures of the test results on the front and the back sides of the plastic film and the topical patch. As shown in FIG. 39A, the topical patch was a slightly concave at the vagina-facing edge and a slightly convex at the opposite edge. It also had a round reinforcing layer laminated on the top surface of the support layer. The topical patch was relatively thin, flat, flexible, and conformable. The support layer had a hypoallergenic or biocompatible adhesive on the bottom surface, which was protected by a release liner. Both the support layer and the round reinforcing layer were made of the same woven fabric tape. FIG. 39B shows a closed-up picture of the custom plastic film model of a dilated vagina with a midline tear propagated into the topical patch affixed onto the perineal area closer to the anus and covering the anus. The topical patch was intentionally not placed adjacent to the edge of the dilated vagina in order to allow adequate vaginal dilation in actual childbirth delivery. It should also be noted that the woven fabric used in this experiment is more tear-resistant in one direction than the other, which is about 90 degrees apart. The topical patch was made with the appropriate orientation of the woven fabric in order to maximize its tear resistance. FIG. 39C shows the back side of the plastic film model and the affixed topical patch. It appears that the medial or midline tear propagated and slightly penetrated into the support layer of the topical patch, but was eventually stopped. Furthermore, the tear appeared to have been deflected laterally by the reinforcing layer as shown in FIG. 39D. Under high stress, the additional tear resistance provided by the reinforcing layer appears to be effective in further resisting tear or deflecting tear. The tear resistance of the support layer and the reinforcing layer appears to be necessary in the present application due to the high stress and high strain conditions that perineal tissues experience during childbirth delivery. Such high stress and strain conditions are not expected for other adhesive patch applications (e.g. transdermal patches, bandages, etc.).

In one aspect of the invention, as shown in FIGS. 9 and 11, topical devices are configured to adhesively attach, affix, bond, adhere or secure to, at least, part of the perineum, which includes the perivaginal area, the perianal area and the area between the vagina and the anus, without covering the anal aperture in order to reduce, limit or prevent perineal laceration. The topical devices can be topical patches comprising a support layer, an adhesive layer, and a release liner as described below. Methods for using such topical devices or patches to reduce, limit or prevent perineal laceration are also contemplated. Topical patches of appropriate configurations when secured onto part of the perineum between the vagina and the anus may reduce, limit or prevent laceration, especially midline or medial laceration. Appropriate patch configurations include appropriate geometry, size, thickness, flexibility, mechanical properties, support layer material, reinforcement materials of the support layer, reinforcement patterns of the support layer, adhesive layer materials, reinforcement of adhesive layer materials, overall physical and mechanical behaviors of the patches, etc. Topical patches of appropriate configurations when adhesively attached, affixed, bonded, adhered or secured onto the perineum region right above the anus but below the vagina (orientation according the views shown by FIGS. 1 and 2) may reduce, limit or prevent a midline or medial episiotomy cut from extending further toward the anus, thereby may reduce, limit or prevent third or fourth degree laceration, which is a common complication associated with medial episiotomy.

The topical devices or patches described above are made of tear-resistant materials and may further be reinforced with similar or preferably better tear-resistant materials or elements in order to support underlying perineal tissues against tear or laceration. The topical patches can be made with materials or methods such that they are stretchable, semi-stretchable or substantially non-stretchable. However, it may be preferred that the topical patches are only semi-stretchable or substantially non-stretchable for more effective protection of underlying tissues. For a stretchable topical patch, it is preferred that the device has a maximum elongation of less than 100% of its original non-stretched length. In the case of a semi-stretchable topical patch, it is preferred that the device has a maximum elongation of less than 50%. A substantially non-stretchable patch is preferred to have less than 20% elongation for the scope of this invention. Depending on the purpose of the topical patches, they can be offered in a variety of configurations. Topical patches of appropriate configurations such as a convex edge facing the vagina and diverging toward the anus (FIGS. 7, 9A, 10A, 14, 15C, 15E, 15 F, 16A, 16B, 16G, 17B, 17F, 18, 19, 20, etc.) may help divert, deflect or steer laceration from a medial direction to a mediolateral direction or bias a diverted mediolateral laceration toward a pre-determined side or direction. In the case of topical patches with a substantially straight edge facing the vagina (FIGS. 11, 13, 15D, 15G, 16H, 17D, 17H, etc.), the tear is expected to be resisted or stopped from propagating toward the anus. Other appropriate configurations of topical patches such as a convex edge connected with relatively straight edges (FIGS. 16B, 16C, 16D, 16G) may divert laceration to mediolateral direction and then limit it from further propagation after extending to a certain length. Topical patches with a concave edge facing the vagina (FIGS. 12, 16E, 16F, 17A, 27, 28, 29, 30, 31, 32, 34, 35, 36, 37, etc.) are expected to be effective in resisting or preventing tear from propagation toward the anus.

At least part of the support layer is made of a tear-resistant material to provide tear-resistant support to the perineal tissues. The tear-resistant material may also provide resistance to excessive stretching to the perineal tissues. The tear-resistant materials can be any synthetic or natural materials including polymers, metals, ceramics or their combinations. The tear resistant materials can be in any shapes or forms including solid or porous structure/construct/formation with a three dimensional shape; solid or porous planar sheet/film/membrane; fibrous, woven, non-woven, braided or knitted textile constructs; assembly, laminate, composite, combination of components or materials; etc. In the case of a textile-based or fabric support layer, an appropriate weave or knit pattern may be selected for suitable levels of tear resistance and stretchability. The support layer may be semi-stretchable with good tear resistance. Alternately, the support layer may be substantially non-stretchable with high tear resistance. However, it may be preferred that the support layer is semi-stretchable with high tear resistance. Examples of fabric adhesive tape that can be used for the support layer include Tricot fabric, knitted fabric, woven fabric, non-woven mesh, etc. Examples of the tear-resistant materials for the support layer include polyester, polyethylene, ultra-high molecular weight polyethylene, polypropylene, polyethylene terephthalate, rayon, cellulose, polyetheretherketone, polyethersulfone, polyvinylidene fluoride, polytetrafluoroethylene, silicone, polyurethane, polyimide, polyamide, or their combinations.

It may also be preferred that, at least, a portion of the support layer is further reinforced with reinforcement members or reinforcement layers for even more tear resistance. Such reinforcements may be achieved by embedded into or laminated onto the support layer with one or more high tear-resistant materials, which include polymers, metals, ceramics and their combinations. Examples of high tear-resistant materials include but not limited to polymeric materials with improved inherent toughness, polymeric materials oriented axially or bi-axially, textile-based or fabric materials, composite polymeric materials, composites of polymers and metals, composites of polymers and ceramics, composite of metals and ceramics, composites of polymers, metals and ceramics, etc. Examples of textile-based materials include fibers, filaments, threads, tethers, tapes, strips, knitted fabric, woven fabrics, non-woven meshes, braided constructs, sheets, films, bars, rods, which can be made of one or more materials selected from polymers, metals, ceramics or their composites. These textile-based materials may be embedded, coated, laminated, bonded, attached or secured to one or more materials (e.g. polymeric matrix materials, polymer films, polymer adhesive tapes, polymer foam layers, absorbent materials, other textile-based materials, polymeric adhesives, or their combinations) to form the support layer, the reinforcement layer or the topical device. In the case of a textile-based or fabric reinforcement layer, an appropriate weave or knit pattern may be selected for suitable levels of tear resistance and stretchability. The reinforcement layer may be semi-stretchable with good tear resistance. Alternately, the reinforcement layer may be substantially non-stretchable with high tear resistance. However, it may be preferred that the reinforcement layer is semi-stretchable with high tear resistance. Examples of fabric adhesive tape that can be used for the reinforcement layer include Tricot fabric, knitted fabric, woven fabric, non-woven mesh, reinforced fabric, etc. Examples of the tear-resistant materials used for the reinforcement layer or reinforcement member include polyester, polyethylene, ultra-high molecular weight polyethylene, polypropylene, polyethylene terephthalate, rayon, cellulose, polyetheretherketone, polyethersulfone, polyvinylidene fluoride, polytetrafluoroethylene, silicone, polyurethane, polyimide, polyamide, or their combinations.

The support layer may have other beneficial features such as peeling tabs or strips not coated with adhesive, water resistance, water-permeability, air-permeability, drug-eluting capability, drug reservoirs, electrical stimulators, micro-needles for transdermal drug delivery, etc. A variety of additives may be incorporated into the support layer or its surfaces for different purposes. Additives can also be incorporated into the adhesive layer or between the support layer and the adhesive layer. Micro-needles may also be included for transdermal delivery of additives. Examples of additives include chemical, pharmacological or biological agents or substances for enhancing the patch performance or providing therapeutic treatment. Examples of pharmaceutical additives include analgesic drugs, anesthetic drugs, anti-inflammatory drugs, antibiotic drugs, etc. Examples of chemical additives include various types of oils, solvents, alcohols, ethanol, glycerol, dimethyl sulfoxide, silver, silver compounds, micro-particles or nano-particles of silver, anti-microbial metals, anti-microbial compounds, anti-microbial solvents, anti-fouling agents, etc.

The adhesive layer bonds or secures the support outer layer to the skin of the patients, and thereby may transfer the tensile and shear stresses from the underlying dermal tissues to the support outer layer. The tensile and shear stresses occurring in the underlying dermal tissues and the topical patches may be substantially parallel to the perineum plane, which is tangent or parallel to the perineum and substantially transverse to the sagittal plane. The adhesive layer preferably has adequate bonding strength to the skin or stress-resisting capability for sufficient resistance to unintended loosening, peeling, loss of adhesion, detaching or delamination due to stresses imposed by underlying dermal tissues. On the other hand, the adhesive layer is preferably easy to be removed or peeled off at the end of its use. Any type of hypoallergenic or biocompatible adhesive materials may be used. It is preferred that the adhesives and their components are non-sensitizing, non-irritating, non-cytotoxic, and biocompatible. Pressure sensitive adhesives are preferred as these pre-coats are easy to use and convenient for the users. However, other types of adhesive are also contemplated, which include self-curing single-component adhesives, self-curing multiple-components adhesives, solvent-evaporation-activated adhesives, air-activated adhesives, moisture-activated adhesives, heat-activated adhesives, light-activated adhesives, uv-activated adhesives, infrared-activated adhesives, radiation-activated adhesives, radio-frequency activated adhesives, etc. Examples of pressure sensitive adhesives include acrylic adhesives, modified-acrylic adhesives, acrylate-based adhesives, silicone adhesives, rubber-based adhesives, etc.

The adhesive layer may partially or totally cover the outer support layer. There may be a variety of adhesive coating patterns on the outer support layer. Examples of adhesive coating patterns include at or around the perimeter, at or around the center, on one or more sides or corners, uniform and complete coating, non-uniform and incomplete coating, random non-continuous coating, complete coating except the peeling tabs, etc. The adhesive layer may have other beneficial features such as water proof, body fluid resistance, water permeability, gas permeability, drug-eluting, drug reservoirs, electrical stimulation, etc. A variety of additives may be incorporated into the adhesive layer or its surface for different purposes. Examples of additives include chemical, pharmacological or biological agents or substances for enhancing the patch performance or providing therapeutic treatment. Examples of pharmaceutical additives include analgesic drugs, anesthetic drugs, anti-inflammatory drugs, antibiotic drugs, etc. Examples of chemical additives include various types of oils, solvents, alcohols, ethanol, glycerol, dimethyl sulfoxide, silver, silver compounds, micro-particles or nano-particles of silver, anti-microbial metals, anti-microbial compounds, anti-microbial solvents, anti-fouling agents, etc.

The release liner is used to protect the adhesive layer prior to use. The release liner is typically removed and discarded right before the topical patch is applied to the perineum. The release liner can be made of a variety of suitable materials and has any suitable size or shape to protect the adhesive layer during manufacturing, packaging, storage, shipping, handling, and application. Examples of the release liner include polymer sheets or films, polymer-coated papers, laminates of paper and polymeric materials, etc. Additional examples of release liners include polyethylene terephthalate (PET) films, densified kraft paper, polycoated kraft, and polyethylene or polypropylene films. Examples of materials used to coat or make release liners include silicone, polyester, polyethylene, polyethylene terephthalate, etc. Materials, designs, and methods of use of release liners are well known to people with skills in the art.

In another aspect of the invention, as shown in FIGS. 7, 14, 26C through 26F, 27C through 27F, and 28C through 28F, topical devices or topical patches are configured to adhesively attach, affix, bond or secure to at least part of the perineum and completely or substantially surround the anus without covering the anal aperture in order to reduce, limit or prevent perineal laceration and anal tissue straining, respectively. The topical devices or topical patches comprise a support outer layer, an adhesive layer, and a release liner as described above and elsewhere in the document. Methods for using such topical patches to reduce, limit or prevent perineal laceration and anal tissue straining are also contemplated. Depending on the configurations and placement of the topical patches, they may reduce, limit or prevent perineal laceration according to the various possible scenarios discussed above and elsewhere in the document. With appropriate configurations that completely or substantially surround the anus, topical patches may be able to resist radial expansion of the anus in order to keep the anal aperture from excessive opening during the pushing process of labor. This may be possible due to a stress transfer mechanism from underlying dermal tissues to the topical patch as shown in FIG. 8. Under stresses caused by the pushing process, the anal sphincter muscle may be strained and the anal aperture may be forced to spread open. Prolonged straining of the sphincter or prolonged opening of the anal aperture may be a cause of tissue damages discussed above. Topical patches with configurations that substantially surrounds the anus, preferably those completely surrounds the anus, may be able to counter the outward radial stresses along the perineum plane that are associated with a strained anal sphincter and an opening anal aperture. As a result, such topical patches may help reduce, limit or prevent anal tissue straining or extrusion.

In yet another aspect of the invention, as shown in FIGS. 10, 12, 13, 26A, 26B, 26G, 26H, 27A, 27B, 27G, 27H, 28A, 28B, 28G, 28H and 29, topical devices or topical patches are configured to adhesively bond to at least part of the perineum, completely or substantially surround the anus and cover the anal aperture in order to reduce, limit or prevent perineal laceration, anal tissue straining, and anal tissue extrusion, respectively. The topical devices or topical patches comprise a support outer layer, an adhesive layer, and a release liner as described above and elsewhere in the document. Methods for using such topical patches to reduce, limit or prevent perineal laceration, anal tissue straining and anal tissue extrusion are also contemplated. Depending on the configurations and placement of the topical patches, they may reduce, limit or prevent perineal laceration according to the various possible scenarios discussed above and elsewhere in the document. With appropriate configurations that completely or substantially surround the anus, topical patches may be able to resist radial expansion of the anus in order to keep the anal aperture from excessive opening during the pushing process of labor. This may be possible due to a stress transfer mechanism from underlying dermal tissues to the topical patch as shown in FIG. 8. Under stresses caused by the pushing process, the anal sphincter muscle may be strained and the anal aperture may be forced to spread open. Prolonged straining of the anal sphincter or prolonged opening of the anal aperture may be a cause of tissue damages discussed above. Topical patches with configurations that substantially surround and cover the anus, preferably those completely surrounds the anus and substantially covers the anal aperture, may be able to counter the outward radial stresses along the perineum plane that are associated with a strained anal sphincter and an opening anal aperture. As a result, such topical patches may help reduce, limit or prevent anal tissue straining and extrusion.

In other aspects of the invention, topical devices or topical patches of appropriate configurations (including shape, size, thickness, materials, reinforcements, etc.) may reduce, limit or prevent perineal lacerations, which include midline or medial laceration and mediolateral laceration, when bonded, adhered or adhesively secured to appropriate locations on the perineum. Topical patches with a shape, size and thickness that are suitable for adhering to, at least, part of the perineum between the vagina and the anus may be used. Examples of such topical devices or patches are shown in FIG. 7 and FIGS. 9 through 37. Topical patches are preferably placed in such a way that they, at least, partially cover the midline on the perineum connecting the vagina and the anus as shown in FIGS. 7, 9 through 14, and 32 through 37. The support layer preferably has adequate tear resistance to help underlying dermal tissues against medial laceration. The adhesive layer preferably has adequate bonding strength to hold the support layer to the underlying dermal tissues under stresses. It may also be preferred that, at least, part of the support layer is further reinforced for tear resistance. Such reinforcements may be achieved by introducing into or onto the support layer one or more high tear-resistant materials, which include polymers, metals, ceramics and their combinations. Examples of high tear-resistant materials include but not limited to polymeric materials with improved inherent toughness, polymeric materials oriented axially or bi-axially, textile-based materials, composite polymeric materials, composites of polymers and metals, composites of polymers and ceramics, composite of metals and ceramics, composites of polymers, metals and ceramics, etc. Examples of textile-based materials include fibers, filaments, threads, tethers, tapes, strips, knitted fabrics, woven fabrics, woven meshes, non-woven meshes, braided constructs, which can be made of one or more materials of polymers, metals or ceramics.

It is preferred that the patches have a shape or configuration with the vagina-facing edge (or the edge's tangent line) forming an angle of less than 180 degrees, preferably less than 165 degrees, more preferably less than 135 degrees from the midline connecting the vagina and the anus as shown in FIGS. 7, 9 through 14, and 32 through 37. A patch with the vagina-facing edge (or the edge's tangent line) that is substantially perpendicular (e.g. 90+/−30 degrees) to the midline connecting the vagina and the anus (FIGS. 11, 13, 15D, 15G, 16E, 16H, 17A, 26 through 37) is preferred. A patch with the vagina-facing edge that is concave such as those in FIGS. 12, 16E, 16F, 17A, 27 through 32, and 34 through 37 is also preferred. Furthermore, a patch with an elongated configuration that is substantially perpendicular to the midline connecting the vagina and the anus such as those in FIGS. 11, 12, 20G, 20H, and 26 through 37 is preferred. It may also be preferred that the patch has extended or expanded corners such as those in FIGS. 12, 16E, 16F, 16H, 17D,17H, 27 through 32, and 34 through 37. The section of the support layer that is located in the vicinity of the midline connecting the vagina and the anus when affixed to the perineum is preferred to have a similar or higher tear resistance than the rest of the patch. The vagina-facing edge is preferred to have a similar or higher tear resistance than the other edges of the patch. It is preferred that the reinforcement layer or element is located within the patch at or near the midline connecting the vagina and the anus when affixed to the perineum. It is also preferred that the reinforcement layer or element is placed at or near the vagina-facing edge of the patch. The reinforcement layer or element may cover the entire support layer. It may be preferred that the tear resistance of the topical devices or patches increases from the vagina-facing edge toward the center or the opposite edge. Such a gradient in tear resistance may be achieved by modifying the designs, structures or materials of the support layer, the reinforcement layer or the overall topical patch. For example, a topical patch with an increasing number or density of reinforcement threads or filaments from the vagina-facing etch to the opposite edge is expected to provide a gradient tear resistance. Gradient tear resistance can be of any patterns (e.g. linear increase or decrease, non-linear increase or decrease, increase then decrease, gradual changes, abrupt changes), of any directions (e.g. one direction, multiple directions, radial direction, random directions), etc.

Topical patches of appropriate configurations as discussed above when affixed, adhered or adhesively secured to appropriate locations on the perineum may reduce, limit or prevent a medial or mediolateral episiotomy cut from extending further toward the anus. Topical patches with a shape, size and thickness that are suitable for adhering to, at least, part of the perineum between the vagina and the anus may be used. Examples of such topical devices or patches are shown in FIG. 7 and FIGS. 9 through 37. Topical patches are preferably placed in such a way that they, at least, partially cover the midline connecting the vagina and the anus as shown in FIGS. 7, 9 through 14, and 32 through 37. It is preferred that topical patches have an appropriate size and shape that can be placed in such a way that one edge of the patch is close to or borders the end of the episiotomy cut on the perineum. This may help resist further propagation or advancement of the episiotomy cut toward the anus and the underlying anal sphincter. It is preferred that the patches have a shape or configuration with the vagina-facing edge (or the edge's tangent line) forming an angle of less than 180 degrees, preferably less than 165 degrees, more preferably less than 135 degrees from the midline connecting the vagina and the anus as shown in FIGS. 7, 9 through 14, and 32 through 37. A patch with the vagina-facing edge (or the edge's tangent line) that is substantially perpendicular to the midline connecting the vagina and the anus (FIGS. 11, 13, 15D, 15G, 16E, 16H, 17A, 26 through 37) is preferred. A patch with the vagina-facing edge that is concave such as those in FIGS. 12, 16E, 16F, 17A, 27 through 32, and 34 through 37 is also preferred. Furthermore, a patch with an elongated configuration that is substantially perpendicular to the midline connecting the vagina and the anus such as those in FIGS. 11, 12, 20G, 20H, and 26 through 37 is preferred. It may also be preferred that the patch has extended or expanded corners such as those in FIGS. 12, 16E, 16F, 16H, 17D,17H, 27 through 32, and 34 through 37. The section of the support layer that is located in the vicinity of the midline connecting the vagina and the anus when affixed to the perineum is preferred to have a similar or higher tear resistance than the rest of the patch. The vagina-facing edge is preferred to have a similar or higher tear resistance than the other edges of the patch. It is preferred that the reinforcement layer or element is located within the patch at or near the midline connecting the vagina and the anus when affixed to the perineum. It is also preferred that the reinforcement layer or element is placed at or near the vagina-facing edge of the patch. As described above, it may be preferred that the tear resistance of the topical devices or patches increases or changes from the vagina-facing edge toward the center or the opposite edge.

Topical patches of appropriate configurations as discussed above when bonded, adhered or adhesively secured to appropriate locations on the perineum may reduce, limit or prevent the third or fourth degree laceration. Topical patches with a shape, size and thickness that are suitable for adhering to, at least, part of the perineum between the vagina and the anus may be used. Examples of such topical devices or patches are shown in FIG. 7 and FIGS. 9 through 37. Topical patches are preferably placed in such a way that they, at least, partially cover the midline connecting the vagina and the anus as shown in FIGS. 7, 9 through 14, and 32 through 37. It is preferred that topical patches have an appropriate size and shape that can be placed in such a way that, at least, part of the patch is right above the underlying anal sphincter muscle and the rectal mucosa in order to protect them against laceration.

Topical patches of appropriate configurations as discussed above when bonded, adhered or adhesively secured to appropriate locations on the perineum may also divert, deflect, steer or bias laceration from a medial direction to a mediolateral direction. Topical patches with a shape, size and thickness that are suitable for adhering to, at least, part of the perineum between the vagina and the anus may be used. Examples of such topical devices or patches are shown in FIGS. 7, 9, 10, 14, 15C, 15E, 15F, 16A, 16B, 16G, 17E, 17F, and 19 through 21. Topical patches are preferably placed in such a way that they, at least, partially cover the midline connecting the vagina and the anus as shown in FIGS. 7, 9 through 14, and 32 through 37. It is preferred that topical patches have a shape with, at least, one of their edges forming an angle of less than 180 degrees, preferably less than 165 degrees from the midline connecting the vagina and the anus as shown in FIG. 7 and FIGS. 9 through 14. The patches may be placed in such a way that propagation or advancement of a midline laceration would tend to follow either the left or the right edge of the patch toward a mediolateral direction in order to help divert or deflect a laceration initiated from the vagina.

Topical patches of appropriate configurations as discussed above when bonded, adhered or adhesively secured to appropriate locations on the perineum may divert, deflect, steer or bias a laceration toward a pre-determined mediolateral direction. Topical patches with a shape, size and thickness that are suitable for attaching, affixing, or adhering to, at least, part of the perineum between the vagina and the anus may be used. Examples of such topical devices or patches are shown in FIGS. 7, 9, 10, 14, 15C, 15E, 15F, 16A, 16B, 16G, 17E, 17F, and 19 through 21. Topical patches are preferably placed in such a way that they, at least, partially cover the midline connecting the vagina and the anus as shown in FIGS. 7, 9, 10, and 14. It is preferred that the patches have a shape with one of their edges forming an angle of less than 180 degrees, preferably less than 165 degrees from the midline connecting the vagina and the anus as shown in FIGS. 7, 9, 10, and 14. The patches may be placed in such a way that propagation or advancement of a midline laceration would follow the edge of the patch toward a predetermined mediolateral direction in order to help divert or deflect a laceration initiated from the vagina.

Topical patches of appropriate configurations as discussed above when bonded, adhered or adhesively secured to appropriate locations on the perineum may divert, deflect, steer or bias laceration to a mediolateral or lateral direction and then limit it from further propagation after its extension to a certain length. Topical patches with a shape, size and thickness that are suitable for adhering to, at least, part of the perineum between the vagina and the anus may be used. Examples of such topical devices or patches may be shown in FIGS. 16A through 16D, 16G, 20G, 20H, 27 through 31. Topical patches are preferably placed in such a way that they, at least, partially cover the midline connecting the vagina and the anus as shown in FIGS. 7, 9 through 14, and 32 through 37. It is preferred that the patches have a shape with their edges forming an angle of less than 180 degrees, preferably less than 165 degrees from the midline connecting the vagina and the anus as shown in FIGS. 7, 9 through 14, and 32 through 37. The patches may be placed in such a way that propagation or advancement of a midline laceration would tend to follow either the left or the right edge of the patch toward a mediolateral or lateral direction in order to help divert or deflect laceration before it reaches a limiting point along the edges of the patch. Such a limiting point may be achieved by adding a more transverse or concave edge to the initial left or right edge of the patch where the laceration may tend to follow during its initial propagation.

In other aspects of the invention, methods are contemplated for using topical patches of appropriate configurations including those discussed above in order to reduce, limit or prevent perineal laceration, which include midline or medial laceration and mediolateral laceration. The methods include providing at least one topical patch with appropriate configurations, adhering or adhesively securing the topical patch to an appropriate location on the perineum, and maintaining the patch during, at least, part of the childbirth labor process in order to reduce, limit or prevent perineal laceration. Topical patches with a shape, size and thickness that are suitable for adhering, attaching, affixing, bonding, or adhesively securing to, at least, part of the perineum between the vagina and the anus may be used according to the described methods. Examples of such topical devices or patches are shown in FIG. 7 and FIGS. 9 through 37. Topical patches are preferably placed in such a way that they, at least, partially cover the midline on the perineum connecting the vagina and the anus as shown in FIGS. 7, 9 through 14, and 32 through 37. The support layer preferably has adequate tear resistance to help underlying dermal tissues against medial laceration. The adhesive layer preferably has adequate bonding strength to hold the support layer to the underlying dermal tissues under stresses. It may also be preferred that, at least, part of the support layer is further reinforced for tear resistance. Such reinforcements may be achieved by introducing into the support layer one or more high tear-resistant materials, which include polymers, metals, ceramics and their combinations. Examples of high tear-resistant materials include but not limited to polymeric materials with improved inherent toughness, polymeric materials oriented axially or bi-axially, textile-based materials, composite polymeric materials, composites of polymers and metals, composites of polymers and ceramics, composite of metals and ceramics, composites of polymers, metals and ceramics, etc. Examples of textile-based materials include fibers, filaments, woven fabrics, non-woven meshes, braided constructs, which can be made of one or more materials of polymers, metals or ceramics.

Methods for using topical patches of appropriate configuration to reduce, limit or prevent medial or mediolateral episiotomy cut from extending further toward the anus are also contemplated. The methods include providing at least one topical patch with appropriate configurations, adhering or adhesively securing the topical patch to an appropriate location on the perineum, and maintaining the patch during, at least, part of the childbirth labor process in order to reduce, limit or prevent a medial or mediolateral episiotomy cut from extending further toward the anus. Topical patches with a shape, size and thickness that are suitable for adhering to, at least, part of the perineum between the vagina and the anus may be used according to the described methods. Examples of such topical devices or patches are shown in FIG. 7 and FIGS. 9 through 37. Topical patches are preferably placed in such a way that they, at least, partially cover the midline on the perineum connecting the vagina and the anus as shown in FIGS. 7, 9 through 14, and 32 through 37. It is preferred that topical patches have an appropriate size and shape that can be placed in such a way that one edge of the patch is close to or borders the end of the episiotomy cut on the perineum. This may help resist further extension, propagation or advancement of the episiotomy cut toward the anus and the underlying anal sphincter.

Methods for using topical patches of appropriate configurations to reduce, limit or prevent the third or fourth degree laceration are also contemplated. The methods include providing at least one topical patch with appropriate configurations, adhering or adhesively securing the topical patch to an appropriate location on the perineum, and maintaining the patch during, at least, part of the childbirth labor process in order to reduce, limit or prevent the third or fourth degree laceration. Topical patches with a shape, size and thickness that are suitable for adhering to, at least, part of the perineum between the vagina and the anus may be used according to the described methods. Examples of such topical devices or patches are shown in FIG. 7 and FIGS. 9 through 37. Topical patches are preferably placed in such a way that they, at least, partially cover the midline on the perineum connecting the vagina and the anus as shown in FIGS. 7, 9 through 14, and 32 through 37. It is preferred that topical patches have an appropriate size and shape that can be placed in such a way that, at least, part of the patch is right above the underlying anal sphincter muscle and the rectal mucosa in order to protect them against laceration.

Methods for using topical patches of appropriate configurations as discussed above to divert, deflect, steer, redirect or bias laceration from a medial direction to a mediolateral direction. The methods include providing at least one topical patch with appropriate configurations, adhering or adhesively securing the topical patch to an appropriate location on the perineum, and maintaining the patch during, at least, part of the childbirth labor process in order to divert, deflect, steer, redirect or bias laceration from a medial direction to a mediolateral direction. Topical patches with a shape, size and thickness that are suitable for adhering to, at least, part of the perineum between the vagina and the anus may be used according to the described methods. Examples of such topical devices or patches are shown in FIGS. 7, 9, 10, 14, 15C, 15E, 15F, 16A, 16B, 16G, 17E, 17F, and 19 through 21. Topical patches are preferably placed in such a way that they, at least, partially cover the midline on the perineum connecting the vagina and the anus as shown in FIG. 7 and FIGS. 9 through 14. It is preferred that topical patches have a shape with, at least, one of their edges forming an angle of less than 180 degrees, preferably less than 165 degrees from the midline connecting the vagina and the anus as shown in FIG. 7 and FIGS. 9 through 14. The patches may be placed in such a way that propagation of a midline laceration would tend to follow either the left or the right edge of the patch toward a mediolateral direction in order to help divert or deflect a laceration initiated from the vagina.

Methods for using topical patches of appropriate configurations as discussed above to divert, deflect, steer, redirect or bias a laceration toward a pre-determined mediolateral direction. The methods include providing at least one topical patch with appropriate configurations, adhering or adhesively securing the topical patch to an appropriate location on the perineum, and maintaining the patch during, at least, part of the childbirth labor process in order to divert, deflect, steer, redirect or bias a laceration toward a pre-determined mediolateral direction. Topical patches with a shape, size and thickness that are suitable for adhering to, at least, part of the perineum between the vagina and the anus may be used according to the described methods. Examples of such topical devices or patches may be shown in FIGS. 7, 9, 10, 14, 15C, 15E, 15F, 16A, 16B, 16G, 17E, 17F, and 19 through 21. Topical patches are preferably placed in such a way that they, at least, partially cover the midline connecting the vagina and the anus as shown in FIGS. 7, 9, 10, and 14. It is preferred that the patches have a shape with one of their edges forming an angle of less than 180 degrees, preferably less than 165 degrees from the midline connecting the vagina and the anus as shown in FIGS. 7, 9, 10, and 14. The patches may be placed in such a way that propagation of a midline laceration would follow the edge of the patch toward a pre-determined mediolateral direction in order to help divert or deflect a laceration initiated from the vagina.

Methods for using topical patches of appropriate configurations as discussed above to divert, deflect, steer, redirect or bias laceration to a mediolateral or lateral direction and then limit it from further propagation after its extension to a certain length. The methods include providing at least one topical patch with appropriate configurations, adhering or adhesively securing the topical patch to an appropriate location on the perineum, and maintaining the patch during, at least, part of the childbirth labor process in order to divert, deflect, steer, redirect or bias laceration to a mediolateral direction and then limit it from further propagation after its extension to a certain length. Topical patches with a shape, size and thickness that are suitable for adhering to, at least, part of the perineum between the vagina and the anus may be used according to the described methods. Examples of such topical devices or patches may be shown in FIGS. 16A through 16D, 16G, 20G, 20H, 27 through 31. Topical patches are preferably placed in such a way that they, at least, partially cover the midline connecting the vagina and the anus as shown in FIGS. 7, 9 through 14, and 32 through 37. It is preferred that the patches have a shape with their edges forming an angle of less than 180 degrees, preferably less than 165 degrees from the midline connecting the vagina and the anus as shown in FIGS. 7, 9 through 14, and 32 through 37. The patches may be placed in such a way that propagation of a midline laceration would tend to follow either the left or the right edge of the patch toward a mediolateral or lateral direction in order to help divert or deflect laceration before it reaches a limiting point along the edges of the patch. Such a limiting point may be achieved by adding a more transverse or concave edge to the initial left or right edge of the patch where the laceration may tend to follow during its initial propagation.

In still another aspect of the invention, additional methods concerning the use of topical devices or topical patches are provided. A method includes providing a topical patch, exposing the adhesive and then placing the topical patch onto at least part of the perineum, which includes the perivaginal area, perianal area or the area between the vagina and the anus, before or during childbirth delivery. A similar method may also be used to support or treat part of the perineum after childbirth delivery using a topical device or patch that contains an additive as described herein. The method may include removing the release liner to expose the adhesive. If other types of adhesive are used instead of a pressure sensitive adhesive, the adhesive coating on the support layer may be activated using a suitable method which includes exposure to environment, a fluid, light, or other sources of energy. The topical patch comprising a support outer layer, an adhesive layer or an adhesive material, and other components may be provided as a kit and may be assembled prior to use. The method may further include preparing the perineum area to receive the topical patch, wherein preparing the perineum area includes shaving, waxing, washing, cleaning, disinfecting, conditioning, treating, anesthetizing, wiping, drying, or combinations thereof. The method may also include removing the release liner in multiple stages or steps during the application of the topical patch. The method may include orienting and positioning the topical patch prior to its placement. The method may include placement of the topical patch on the lower or posterior side of the perineal area, which is closer to the anus than the vagina, in order to reduce potential interference with vaginal dilation. The method may include using additional adhesive or adhesive tapes to further secure or reinforce the topical patch. The method may include support of the topical patch by a hand or by an applicator pushing onto the device during, at least, part of the childbirth delivery process. The method may include removing the patch after childbirth delivery. The method may include dressing or supporting the patient's compromised perineum with the topical patch after childbirth delivery. The method may include dressing or treating the patient's compromised perineum with the topical patch after childbirth delivery, wherein the patch contains one or more additives or active agents described elsewhere in the document. The method may include treating sensitive or painful perineum after childbirth delivery using a topical patch that contains a solvent, a chemical agent, a pharmaceutical agent, and/or a biological agent. The method may include treating sensitive or painful perineum after childbirth delivery using a topical patch that contains one or more analgesic or anesthetic agents or drugs.

It may be preferred that the patch is not placed immediately adjacent to the edge of the dilated vagina as the perineal tissues closest to the vagina should be allowed to stretch as naturally as possible in order to facilitate dilation. The closest edge of the patch to the vagina is preferred to be positioned at an appropriate distance away from the edge of the dilated vagina. Such an appropriate distance depends on the perineal length of the patient, which is the linear measurement between the closest edges of the vagina and the anus. It is preferred that the distance between the closest edges of the patch and the vagina is larger than 20% of the patient's perineal length, preferably larger than 35%, more preferably larger than 50%. The preferred distance may be determined by an obstetrician or a midwife in order to achieve optimal balance between allowance of natural perineal stretching and reduced risks of laceration, unintended extension of an episiotomy cut, third or fourth degree of laceration, etc.

Tears in the anterior area of the vaginal opening may also occur during childbirth delivery, but these lacerations are typically small. The topical devices described above such as those of FIGS. 9, 11, 12, 17A, 17F, 18D, 18G, 21A, 21B, 21C, 26, 27, and 28 may also be used with appropriate orientations to help reduce or prevent tear or laceration of the anterior or the vagina. In such an application, the topical devices may be placed in the vicinity of the anterior area of the vagina or the mons pubis.

The method concerning the use of topical devices or topical patches may optionally include additional support by an applicator to: 1) facilitate placement and bonding of the topical patch onto the perineum prior to or during childbirth delivery; 2) improve or maintain adhesive bonding of the topical patch to the perineum during delivery; 3) prevent premature, loosening, peeling, loss of adhesion, detaching or delamination of the topical patch during delivery; 4) fix, repair or reapply prematurely detached or delaminated topical patch during delivery; or 5) remove the topical patch after delivery. The applicator may optionally be used to support the topical patch against perineal dermal tissues in order enhance the effectiveness of the pressure-sensitive adhesive layer or other types of adhesive layer. The applicator may be held by hand or some how secured to the patient. In one embodiment, the applicator has an elongated shape with the distal end configured to contact or engage the outer surface of the topical patch on the perineum and the proximal end configured for holding with one or both hands. In another embodiment, the applicator has a body with the distal surface configured to contact or engage the topical patch on the perineum and the proximal surface configured to be secured to the patient by a fastening means including belts, straps, tethers, cords, adhesive tapes, hook-and-loop tapes, etc. The applicator, the topical patch and other components may be provided in a single or multiple kits to the medical staff. It is preferred that these components of the product kits are supplied in sterile single-use packaging. The applicator may be provided separately as a single-use or a reusable instrument. Depending on whether the applicator is for single-use or reusable, the instrument is preferred to be made of one or more materials that can withstand at least one type of sterilization methods such as steam, autoclaving, heat, chemical, ethylene gas, gamma radiation, electron-beam radiation. Examples of suitable materials for the applicator include polycarbonate, polyvinyl chloride, acrylics, polyimide, polyamide, polyesters, polyethylene, ultra-high molecular weight polyethylene, polypropylene, polyaryletherketone, polyethersulfone, polyvinylidene fluoride, polytetrafluoroethylene, silicone, polyurethane, stainless steel, titanium alloys, cobalt-chrome alloys, or their combinations.

The topical patch and the applicator may have complementary designs with appropriate configurations and features for achieving one or more of the functions or purposes mentioned above. The outer surface of the topical patch may have features for releasable engagement or securing to the distal end or distal surface of the applicator. Examples of the features include: 1) an adhesive layer on the outer surface of the patch for attaching, affixing, bonding, or adhering to the surface of the applicator, 2) a non-adhesive outer surface of the patch for attaching, affixing, bonding, or adhering to an adhesive surface of the applicator, 3) a patch surface with loops for engaging the surface with hooks on the applicator, 4) a patch surface with hooks for engaging the surface with loops on the applicator, 5) a patch surface with high friction such as textured surface, serrated surface, porous surface, rubbery surface, silicone surface or the alike for friction engagement with a high friction surface on the applicator. The topical patch may be placed on the perineum and subsequently engaged with the applicator for bonding support. The topical patch may be placed on the distal end or distal surface of the applicator before adhesively attaching, affixing, bonding, or securing to the perineum. The method of use may also include direct hand support of the topical patch by a medical staff instead of using the applicator. The support of the topical patch prior or during delivery may be done using one or both hands.

The topical devices or topical patches used in the methods described above comprise a support layer, an adhesive layer, and a release liner. Topical patches of appropriate configurations when adhesively bonded or secured onto the perineum between the vagina and the anus may reduce or limit perineal laceration, especially midline or medial laceration, and other tissue damages. Appropriate patch configurations include appropriate geometry, size, thickness, flexibility, mechanical properties, support layer material, reinforcement materials of the support layer, reinforcement patterns of the support layer, adhesive layer materials, reinforcement materials for the adhesive layer, overall physical and mechanical behaviors of the patches, releasable additives in the patches, etc.

The topical devices or topical patches used in the methods described above may have any geometries or shapes. The topical patches may be two or three-dimensional objects. The edges or surfaces of the topical patches may be flat, linear, curved, angular, twisted, folded, rolled-up, supported or combinations of these characteristics. The edges or surfaces of the topical patches may be concave, convex, wavy, porous, semi-porous, perforated, partially perforated, or combinations of these characteristics. For example, the topical patches may be a substantially planar or two-dimensional device with a width and length (or a diameter if relatively circular) and a thickness substantially small compared to the other dimensions. In another example, the topical patches may be a three-dimensional device with a depth or height (or a relatively large thickness) in addition to a width and length (or a diameter). The upper and lower surfaces of the topical patches may be substantially planar and parallel with each other. However; if the topical patches are flexible, pliable or conformable, their substantially planar shape may change and conform to the shape or topography of the underlying tissue surface to which they adhere. If the topical patches are only partially flexible, pliable or conformable, only the flexible or pliable portion of the topical patches is expected to change and conform to the shape or topography of the underlying tissue surface to which they adhere. The upper or lower surfaces of the topical patches may be three-dimensional and substantially unparallel with each other. The three-dimensional topical patches may be partially or entirely flexible or pliable. As shown in FIG. 25, the topical patches can be concave or convex. The topical patches may also have a variety of other three-dimensional shapes such as a cone, a funnel, a bowl, an inverted bowl, a curved sheet, a pyramid, etc.

The topical devices or topical patches used in the methods described above may have any suitable size for adhesively bonding, adhering or securing onto the perineum. The topical patches should be sized appropriately so that it provides sufficient coverage of the perineum between the vagina and the anus, around the anus, and/or above the anus for protection against laceration and other tissue damages. However, the size of the topical patches should not be excessively large that it would cover too much of the perineum in order to minimize potential interference to the stretching of the perineum for delivery. In the areas that do not significantly interfere with the perineal stretching for delivery, the topical patches may have a larger surface coverage for increased bonding area to the underlying tissues. This is expected to help enhance overall bonding, adhesion or fixation of the topical patches. It is preferred that the largest dimension of the patches does not exceed 400 mm, more preferably less than 300 mm, and more preferably less than 200 mm. In the case of the two-dimensional topical patches, the largest thickness (measured substantially along the sagittal plane) of the patches is preferred to be 10 mm, more preferably less than 5 mm, and more preferably less than 3 mm. The patch thickness may be uniform or variable through out the patch surface. In the case of three-dimensional topical patches, the depth, height or thickness of the topical patches, which may be measured along the sagittal plane, is preferably less than 35 mm, more preferably less than 25 mm, and most preferably less than 15 mm.

The topical devices or topical patches used in the methods described above may be pliable or flexible, semi-flexible, semi-rigid, or rigid. The topical patches may be partially or completely flexible. In the case of partially flexible patches, the flexible portions may be positioned anywhere within the patches. For example, the flexible portions may be at or around the edges, at or around the center of the patches, at one side or one corner of the patches, uniformly or randomly located within the patches, etc. The patches may be inelastic, semi-elastic, elastic or combinations of these characteristics. For examples, the patches may be inelastic where they must resist stretching and may be semi-elastic or elastic where they must allow certain stretching. Unlike transdermal drug-delivery topical patches currently on the market, the topical patches disclosed in this invention must have adequate mechanical properties and tear resistance in order to support the perineal tissues against laceration and other tissue damages. The patches may also have appropriate tensile properties to help support the underlying dermal tissue against excessive tensile stresses or stretching. It may be preferred that, at least, certain parts of the topical patches are further reinforced for high tear resistance and/or high stretching resistance. The reinforcement for the topical patches may be achieved via reinforcement of the support layer and/or the adhesive layer. In one embodiment, the topical patches are resistant to tears caused by perineal tissue laceration or cuts made by surgical instruments such as scalpels or episiotomy scissors. In a different embodiment, the topical patches are resistant to tears caused by perineal tissue laceration but not cuts that are made by surgical instruments such as scalpels or episiotomy scissors. This may be beneficial in the case that the initial episiotomy cut must be extended partially or completely through the topical patches in order to facilitate safe or fast delivery.

A flexible topical device or patch with a large size or large surface area may benefit from a less flexible or more rigid support frame or support element positioned around and/or within the device or its surface. Such a support frame or support element is expected to provide increased rigidity to the flexible topical device, which may be useful for its handling and placement. In one embodiment, the support frame or support element is designed such that it reduces the overall flexibility of the flexible topical patch. In another embodiment, the support frame or support element is designed such that the flexible topical patch does not fold over itself or experiences excessive deformation under its own weight during handling or placement, especially after the release liner is removed. The support frame may have any shapes or configurations, which include elongated shape, ring shape, cross shape, X shape, H shape, I shape, N shape, C shape, U shape, Y shape, W shape, or their combinations. The support frame may be placed at any locations on or within the topical device such as along the periphery, along the diameter, near or across the center, on top of the reinforcement layer, on top and across the reinforcement layer, on top of the support layer, on top of the support layer and around the reinforcement layer, embedded between the reinforcement layer and the support layer, embedded between the support layer and the adhesive layer, between the adhesive layer and the release liner, etc. The support frame may be a permanent or removable component of the topical device. In one embodiment, the support frame may be removed during or after placement of the patch. In another embodiment, the support frame is a non-removable or permanent integral part of the topical patch.

Examples of the support frame include: 1) a X-shaped layer of a pliable foam sheet that is less flexible than the support layer, 2) a H-shaped layer of a pliable polymer sheet that is less flexible than the support layer, 3) a ring-shaped layer of a pliable fabric sheet that is less flexible that the support layer, 4) a strip of a pliable foam layer disposed along the support layer's periphery, 5) a U-shaped layer of a pliable polymer sheet placed along the adhesive layer's periphery and embedded between the adhesive layer and the release liner, 6) at least one polymer or metal strip that is less flexible than the support layer positioned along the length of the topical patch, 6) a pliable foam sheet that is less flexible than the support layer but of the same size and/or shape as the support layer, 7) a combination of example #1 and example #4, 8) a combination of example #4 and example #6. The thickness of the support frame may be in the range from 0.1 mm to 10 mm, preferably from 0.2 mm to 5 mm, more preferably from 0.3 mm to 3 mm. The materials used for the support frame may be a polymer, a metal, a ceramic or their combinations. Examples of polymeric materials include polymer foam sheet, solid polymer sheet, perforated polymer sheet, polymer mesh, woven fabric, knitted fabric, non-woven mesh, or combinations thereof. The support frame can be embedded, attached, affixed, bonded, or laminated to the support layer, the reinforcement layer, and/or the adhesive layer by any appropriate means known in the art including adhesive bonding, thermal fusion, compression molding, ultrasonic welding, sewing, etc.

The support frame may be designed and fabricated such that it can also be used as the tear-resistant reinforcement layer. Similarly, the tear-resistant reinforcement layer may be designed and constructed such that it can also be used as the support frame. Thus, the tear-resistant reinforcement layer with appropriate designs, materials, constructs, positioning within the topical device, etc. may function as a reinforcement member against tear as well as a structural support element for increased rigidity and support of the flexible topical device during handling, especially after the release liner is removed.

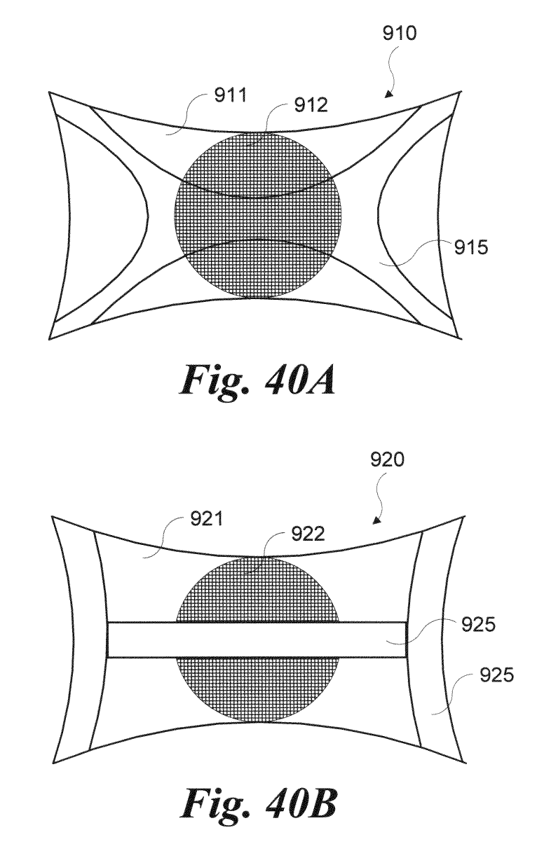

Examples of the support frame described above are shown in FIGS. 40A and 40B. Other configurations of the support frame for the flexible topical device are also contemplated. FIG. 40A shows a flexible topical device or patch 910 with a X-shaped support frame or support element 915. The support frame 915 is laminated on top of the support layer 911 but beneath the reinforcement layer 912. Alternately, the support frame 915 may be laminated on top of both the support layer 911 and the reinforcement layer 912. The support frame 915 may provide increased rigidity to the flexible topical patch 910 in order to improve the handling and/or performance of the device. FIG. 40B shows a flexible topical device 920 with a H-shaped support frame 925. The support frame 925 is affixed or bonded to the top surface of both the support layer 921 and the reinforcement layer 922. The support frame may also be laminated between the support layer 921 and the reinforcement layer 922. Furthermore, the support frame 925 may be embedded or laminated between the support layer 921 and the adhesive layer of the topical patch.

The topical devices or patches described above with tear-resistant capability may also be used to support, protect or treat tissues outside of the perineal or perianal areas. The topical devices may be used to reinforce weakened tissues, support compromised tissues, protect injured tissues, dress sensitive tissues, or treat painful tissues anywhere on an animal or a human subject. The topical devices may be applied or affixed onto any dermal tissues to prevent or limit an initiation or propagation of a stress- or strain-induced tear in a tissue resulting from an incision made therein when the topical devices are disposed on the tissue adjacent to the incision. The incision may be made at the dermal tissues located beneath the topical patches by cutting through the patches or cutting within the opening of the patch.

A variety of additives may be incorporated into the topical patches or their surfaces for different purposes. Examples of additives include chemical, pharmacological or biological agents and substances for enhancing the patch performance or providing therapeutic treatment. Examples of pharmaceutical additives include analgesic drugs, anesthetic drugs, anti-inflammatory drugs, antibiotic drugs, etc. Examples of chemical additives include various types of oils, solvents, alcohols, ethanol, glycerol, dimethyl sulfoxide, etc. The topical patches may have other beneficial features or characteristics such as peeling tabs without adhesive coating, water resistance, body fluid resistance, gas permeability through micro-porosity, gas permeability through macro-porous openings or channels, heating or cooling capability to sooth pain or facilitate healing, etc. The topical patches may be transparent, translucent, opaque or combinations of these optical characteristics. The topical patches may have useful printings on the surface such as dimensional references, linear or angular orientation guiding features, reference position marking features, instructions of use, warnings, and other helpful texts or images. The topical patches may have overhung tabs or strips that are not coated with adhesive in order to help facilitate patch peeling or removal after or during delivery. The topical patches may be further secured to the perineum with additional means including manual pushing, hand pressing, device supporting, adhesive tapes, tethers, straps, belts, hooks-and-loops components, or any other means known by people with skills in the art. The topical patches comprising a support layer, an adhesive layer or an adhesive material, and other optional components mentioned above may be provided as a kit and assembled prior to use.

At least part of the support layer is made of a material with adequate tear resistance, preferably with good to high tear resistance, in order to provide support against tear or laceration to underlying dermal tissues in the perineum. The tear-resistant materials may be any synthetic or natural materials including polymers, metals, ceramics or their combinations. The tear-resistant materials may be in any shapes or forms including solid or porous sheet, film or membrane; fibrous, woven, non-woven, braided or knitted textile constructs; assembly, laminate, composite, combination of components or materials; etc. Examples of the tear-resistant materials for the outer layer include polyesters, polyethylene, ultra-high molecular weight polyethylene, polypropylene, polyetheretherketone, polyethersulfone, polyvinylidene fluoride, polytetrafluoroethylene, silicone, polyurethane, polyimide, polyamide, or their combinations. At least 2%, preferably 10%, and more preferably 25% of the support layer is made of tear-resistant materials. The remaining material of the support layer may be made of any materials suitable for mixing, blending, bonding, joining, laminating, or otherwise contacting the tear-resistant materials. The combinations of the tear-resistant materials and the remaining materials within the support layer can be of any arrangements, patterns, orientations, distributions, or degrees of homogeneity that are suitable for effective support against tear or laceration of underlying dermal tissues in the perineum. For example, tear-resistant materials such as fibers, filaments or fabric may be preferred to be arranged in a substantially perpendicular fashion such as an approximate 90-degrees angle when compared to the medial line between the vagina and the anus as the topical patch is secured to the perineum. Certain combinations of materials within the support layer may be intended to divert tear or laceration toward a different direction such as mediolateral direction from an otherwise medial direction. For example, tear-resistant materials such as polymeric fibers, filaments or fabric can be arranged in a substantially angular fashion such as a 45-degrees angle when compared to the medial line between the vagina and the anus as the topical patch is secured to the perineum. In another example, an approximate 45-degrees angle between fabric filaments and the medial line between the vagina and the anus may be preferred if a certain level of patch stretching is desired. The support layer preferably has a shape, size, thickness, and flexibility or rigidity that are suitable for bonding to dermal tissues in the perineum and adjacent areas.

It may also be preferred that, at least, part of the support layer is further reinforced for high tear resistance. Such reinforcements may be achieved by introducing into the support layer one or more high tear-resistant materials, which include polymers, metals, ceramics and their combinations. Examples of high tear-resistant materials include but not limited to polymeric materials with improved inherent toughness, polymeric materials oriented axially or bi-axially, textile-based materials, composite polymeric materials, composites of polymers and metals, composites of polymers and ceramics, composite of metals and ceramics, composites of polymers, metals and ceramics, etc. Examples of textile-based materials include fibers, filaments, woven fabrics, non-woven meshes, braided constructs, which can be made of one or more materials of polymers, metals or ceramics. The support layer may have a thickness of less than 8 mm, preferably less than 4 mm, more preferably less than 2 mm. It may be transparent, translucent or opaque. It may have any colors, patterns, images or text printed on the outer surface. It may be smooth or textured. It may have uniform or variable thickness. It may have uniform or variable density. It may have elastic, semi-elastic, inelastic characteristics or various combinations of these behaviors.

The adhesive layer bonds or secures the support outer layer to the skin of the patients, and thereby may transfer the tensile and shear stresses from the underlying dermal tissues to the support outer layer. The tensile and shear stresses occurring in the underlying dermal tissues and the topical patches may be substantially parallel to the perineum plane, which is tangent or parallel to the perineum and substantially transverse to the sagittal plane. The adhesive layer preferably has adequate bonding strength to the skin or stress-resisting capability for sufficient resistance to unintended loosening, peeling, loss of adhesion, detaching or delamination due to stresses imposed by underlying dermal tissues. On the other hand, the adhesive layer is preferably easy to be removed or peeled off at the end of its use. Any type of hypoallergenic adhesive materials may be used. It is preferred that the adhesives and their components are non-sensitizing, non-irritating, non-cytotoxic, and biocompatible. Pressure sensitive adhesives are preferred as these pre-coats are easy to use and convenient for the users. However, other types of adhesive are also contemplated, which include self-curing single-component adhesives, self-curing multiple-components adhesives, solvent-evaporation-activated adhesives, air-activated adhesives, moisture-activated adhesives, heat-activated adhesives, light-activated adhesives, uv-activated adhesives, infrared-activated adhesives, radiation-activated adhesives, radio-frequency activated adhesives, etc. Examples of pressure sensitive adhesives include acrylic adhesives, modified-acrylic adhesives, acrylate-based adhesives, silicone adhesives, rubber-based adhesives, etc.

The adhesive layer may partially or totally cover the outer support layer. There may be a variety of adhesive coating patterns on the outer support layer. Examples of adhesive coating patterns include at or around the perimeter, at or around the center, on one or more sides or corners, uniform and complete coating, non-uniform and incomplete coating, random non-continuous coating, complete coating except the peeling tabs, etc. The adhesive layer may have other beneficial features such as water proof, body fluid resistance, water permeability, gas permeability, drug-eluting, drug reservoirs, electrical stimulation, etc. A variety of additives may be incorporated into the adhesive layer or its surface for different purposes. A layer of absorbent material may be placed on the adhesive side of the topical patch for a variety of purposes including absorbing moisture, absorbing some anal discharge, holding and delivery of sprayed analgesics, containing and delivery of other additives for therapeutic treatments, etc. The topical patches with an absorbent layer can be used by a medical staff to deliver various sprayed-on formulations of analgesic drugs, local anesthetic drugs, or other additives. Examples of additives include chemical, pharmacological or biological agents or substances for enhancing the patch performance or providing therapeutic treatment. Examples of pharmaceutical additives include analgesic drugs, anesthetic drugs, anti-inflammatory drugs, anti-infective drugs, anti-allergic drugs, antibiotic drugs, etc. Examples of chemical additives include various types of oils, solvents, alcohols, ethanol, glycerol, dimethyl sulfoxide, silver, silver compounds, micro-particles or nano-particles of silver, anti-microbial metals, anti-microbial compounds, anti-microbial solvents, anti-fouling agents, etc.

The release liner is used to protect the adhesive layer prior to use. The release liner is typically removed and discarded right before the topical patch is applied to the perineum. The release liner can be made of a variety of suitable materials and has any suitable size or shape to protect the adhesive layer during manufacturing, packaging, storage, shipping, handling, and application. Examples of the release liner include polymer sheets or films, polymer-coated papers, laminates of paper and polymeric materials, etc. Additional examples of release liners include polyethylene terephthalate (PET) films, densified kraft paper, polycoated kraft, and polyethylene or polypropylene films. Examples of materials used to coat or make release liners include silicone, polyester, polyethylene, polyethylene terephthalate, etc. Materials, designs, and methods of use of release liners are well known to people with skills in the art.

The adhesive layer and/or the support layer may contain one or more additives selected from the group comprising a solvent, a chemical agent, a pharmaceutical agent, a biological agent, or combinations thereof. Pharmacological agents include analgesic drugs, anesthetic drugs, anti-inflammatory drugs, antibiotic drugs, etc. Analgesic drugs include topical analgesics, opioid analgesics, narcotic analgesics, etc. Examples of topical analgesics include rubefacients, nonsteroidal anti-inflammatory drugs (NSAIDs), local anesthetics, and others. Examples of Rubefacients include traditional formulations based on salicylate and nicotinate esters, capsaicin and capsicum extracts and derivatives. Further examples of Rubefacients include benzocaine, benzyl nicotinate, methyl salicylate, diethylamine salicylate, trolamine salicylate, salicylamide, camphor, menthol, dimethyl sulfoxide, etc. Examples of NSAIDs include diclofenac, felbinac, ibuprofen, ketoprofen, piroxicam, naproxen, flurbiprofen and other NSAIDs. Examples of local anaesthetics include lidocaine, lignocaine and benzocaine. Examples of other topical analgesics include benzydamine, mucopolysaccharide polysulphate, salicylamide and cooling spray formulae.

Various non-limiting aspects of the present invention are enumerated, as follows.

1. A method for support of perineal tissues, the method comprising the steps of:

providing a topical device having a support layer on a first side and an adhesive layer on a second side, said topical device configured for adhesive attachment to at least a portion of the perineal area of a human body;

positioning said topical device approximate at least a portion of the perineal area; and

adhesively secure said topical device to at least a portion of the perineal area.

2. The method of aspect 1, further comprising the step of preparing said portion of the perineal area before adhesively securing said topical device.
3. The method of aspect 2, wherein the step of preparing said portion of the perineal area includes shaving, waxing, washing, cleaning, disinfecting, conditioning, treating, anesthetizing, wiping, drying, or combination thereof.
4. The method of aspect 1, wherein said topical device further comprising a release liner for protecting said adhesive layer before application.
5. The method of aspect 4, further comprising the step of removing said release liner prior to adhesively securing said topical device.
6. The method of aspect 1, wherein at least a portion of said topical device is substantially flexible.
7. The method of aspect 1, wherein at least a portion of said topical device is semi-stretchable or substantially non-stretchable.
8. The method of aspect 1, wherein said topical device has a concave edge
9. The method of aspect 8, wherein said concave edge of said topical device faces the vagina.
10. The method of aspect 1, wherein at least a portion of said topical device is substantially tear-resistant.
11. The method of aspect 1, wherein said topical device is substantially tear-resistant, but not cut-resistant.
12. The method of aspect 1, wherein said support layer is made of a polymeric material, a metallic material, a ceramic material, or combination thereof.
13. The method of aspect 12, wherein said polymeric material includes polyesters, polyethylene, ultra-high molecular weight polyethylene, polypropylene, polyetheretherketone, polyethersulfone, polyvinylidene fluoride, polytetrafluoroethylene, silicone, polyurethane, polyimide, polyamide, or their combinations.
14. The method of aspect 1, wherein at least a portion of said support layer is reinforced with a high-tear resistant material.
15. The method of aspect 1, wherein at least a portion of said adhesive layer is a pressure sensitive adhesive.
16. The method of aspect 15, wherein said pressure sensitive adhesive includes acrylic adhesives, modified-acrylic adhesives, silicone adhesives, rubber-based adhesives, or combinations thereof.
17. The method of aspect 1, wherein at least a portion of said release line is made of PET films, densified kraft paper, polycoated kraft, and polyethylene films.
18. The method of aspect 1, wherein said portion of the perineal area includes at least a portion of one of the perivaginal area, the perianal area and the area between the vagina and the anus.
19. The method of aspect 1, further comprising the step of supporting said topical device with an applicator device that comprises an elongated body with a distal end and a proximal end, wherein the distal end is configured for releasably engagement to the outer surface of said topical patch and the proximal end is configured for hand-holding or securing to the patient.
20. The method of aspect 1, further comprising the step of supporting said topical device by one or both hands of a medical staff.
21. A method for limiting perineal laceration during childbirth delivery, the method comprising the steps of:

providing a topical device having a support layer on a first side and an adhesive layer on a second side, said topical device configured for adhesive attachment to at least a portion of the perineal area of a patient;

positioning said topical device approximate at least a portion of the perineal area; and

adhesively secure said topical device to at least a portion of the perineal area.

22. A method for limiting perineal laceration during childbirth delivery, the method comprising the steps of:

providing a topical device having a support layer on a first side and an adhesive layer on a second side, said topical device configured for adhesive attachment to at least a portion of the perineal area between the vagina and the anus of a patient;

positioning said topical device approximate at least a portion of the perineal area between the vagina and the anus; and

adhesively secure said topical device to at least a portion of the perineal area between the vagina and the anus.

23. A method for limiting perineal laceration during childbirth delivery, the method comprising the steps of:

providing a topical device having a support layer on a first side and an adhesive layer on a second side, said topical device configured for adhesive attachment to at least a portion of the perineal area between the vagina and the anus of a patient;

preparing the perineal area to receive said topical device;

positioning said topical device approximate at least a portion of the perineal area between the vagina and the anus; and

adhesively secure said topical device to at least a portion of the perineal area between the vagina and the anus.

24. A method for limiting perineal laceration during childbirth delivery, the method comprising the steps of:

providing a topical device having a support layer on a first side, an adhesive layer on a second side, and a release liner covering the adhesive layer, said topical device configured for adhesive attachment to at least a portion of the perineal area between the vagina and the anus of a patient;

preparing the perineal area to receive said topical device;

removing at least part of said release liner to expose said adhesive layer;

positioning said topical device approximate at least a portion of the perineal area between the vagina and the anus; and

adhesively secure said topical device to at least a portion of the perineal area between the vagina and the anus.

25. A method for limiting perineal laceration and other tissue damages during childbirth delivery, the method comprising the steps of:

providing a topical device having a support layer on a first side and an adhesive layer on a second side, said topical device configured for adhesive attachment to at least a portion of the perineal area between the vagina and the anus and the perineal area around the anus of a patient;

positioning said topical device approximate at least a portion of the perineal area between the vagina and the anus and the perineal area around the anus; and

adhesively secure said topical device to at least a portion of the perineal area between the vagina and the anus and the perineal area around the anus.

26. A method for limiting perineal laceration, tissue straining, and tissue extrusion during childbirth delivery, the method comprising the steps of:

providing a topical device having a support layer on a first side and an adhesive layer on a second side, said topical device configured for adhesive attachment to at least a portion of the perineal area between the vagina and the anus, the perineal area around the anus, and the anus of a patient;

positioning said topical device approximate at least a portion of the perineal area between the vagina and the anus, the perineal area around the anus, and the anus; and

adhesively secure said topical device to at least a portion of the perineal area between the vagina and the anus, the perineal area around the anus, and the anus.

27. A method for limiting episiotomy cut from advancing toward the anus during childbirth delivery, the method comprising the steps of:

providing a topical device having a support layer on a first side and an adhesive layer on a second side, said topical device configured for adhesive attachment to at least a portion of the perineal area between the vagina and the anus of a patient;

positioning said topical device approximate at least a portion of the perineal area between the vagina and the anus; and

adhesively secure said topical device to at least a portion of the perineal area between the vagina and the anus.

28. A method for reducing third and fourth degree perineal laceration during childbirth delivery, the method comprising the steps of:

providing a topical device having a support layer on a first side and an adhesive layer on a second side, said topical device configured for adhesive attachment to at least a portion of the perineal area between the vagina and the anus of a patient;

positioning said topical device approximate at least a portion of the perineal area between the vagina and the anus; and

adhesively secure said topical device to at least a portion of the perineal area between the vagina and the anus.

29. A method for reducing medial laceration of perineal tissues during childbirth delivery, the method comprising the steps of:

providing a topical device having a support layer on a first side and an adhesive layer on a second side, said topical device configured for adhesive attachment to at least a portion of the perineal area between the vagina and the anus of a patient;

positioning said topical device approximate at least a portion of the perineal area between the vagina and the anus; and

adhesively secure said topical device to at least a portion of the perineal area between the vagina and the anus.

30. A method for diverting medial laceration of perineal tissues during childbirth delivery, the method comprising the steps of:

providing a topical device having a support layer on a first side and an adhesive layer on a second side, said topical device configured for adhesive attachment to at least a portion of the perineal area between the vagina and the anus of a patient;

positioning said topical device approximate at least a portion of the perineal area between the vagina and the anus; and

adhesively secure said topical device to at least a portion of the perineal area between the vagina and the anus.

31. A topical device for support of perineal tissues comprising a support layer on a first side and an adhesive layer on a second side, said topical device configured for adhesive engagement to at least a portion of the perineal area of a human body.
32. The device of aspect 31, further comprising a release liner for protecting said adhesive layer before application to said portion of the perineal area.
33. The device of aspect 31, wherein the shape of said topical device can be one of those shown in FIGS. 15 through 31.
34. The device of aspect 31, wherein at least a portion of said topical device is substantially flexible.
35. The device of aspect 31, wherein at least a portion of said topical device is semi-stretchable or substantially non-stretchable.
36. The device of aspect 31, wherein said topical device has a concave edge
37. The device of aspect 36, wherein said concave edge of said topical device faces the vagina
38. The device of aspect 31, wherein at least a portion of said topical device is substantially tear-resistant.
39. The device of aspect 31, wherein said topical device is substantially tear-resistant.
40. The device of aspect 31, wherein said topical device is substantially planar.
41. The device of aspect 31, wherein said topical device is a topical adhesive patch.
42. The device of aspect 31, wherein said support layer is made of a polymeric material, a metallic material, a ceramic material, or combination thereof.
43. The device of aspect 42, wherein said polymeric material includes polyesters, polyethylene, ultra-high molecular weight polyethylene, polypropylene, polyethylene terephthalate, rayon, cellulose, polyetheretherketone, polyethersulfone, polyvinylidene fluoride, polytetrafluoroethylene, silicone, polyurethane, polyimide, polyamide, or their combinations.
44. The device of aspect 31, wherein said support layer is polymeric film, sheet or membrane of a solid or a porous configuration.
45. The device of aspect 31, wherein said support layer is a textile fabric, mesh or sheet of woven, non-woven, braided or knitted configuration.
46. The device of aspect 31, wherein said support layer is a laminate or composite of a textile layer and a polymeric layer, wherein the textile layer and the polymeric layer can be separated or intermingled.
47. The device of aspect 31, wherein at least a portion of said support layer is reinforced or laminated with a high-tear resistant material including high tenacity fibers, high modulus fibers, oriented polymeric films, high-tear resistant polymers in elongated configurations, fabric layers, mesh layers, or combination thereof.
48. The device of aspect 31, wherein at least a portion of said adhesive layer is a hypoallergenic adhesive.
49. The device of aspect 31, wherein at least a portion of said adhesive layer is a pressure sensitive adhesive.
50. The device of aspect 49, wherein said pressure sensitive adhesive includes acrylic adhesives, modified-acrylic adhesives, silicone adhesives, rubber-based adhesives, or combinations thereof.
51. The device of aspect 31, wherein at least a portion of said adhesive layer is reinforced or laminated with a high-tear resistant material including high tenacity fibers, high modulus fibers, oriented polymeric films, high-tear resistant polymers in elongated configurations, fabric layers, mesh layers, or combination thereof.
52. The device of aspect 31, wherein at least a portion of said adhesive layer contains an additive selected from the group comprising a solvent, a chemical agent, a pharmaceutical agent, a biological agent, or combination thereof.
53. The device of aspect 31, wherein at least a portion of said release line is made of PET films, densified kraft paper, polycoated kraft, and polyethylene films.
54. The device of aspect 31, further comprising an applicator device for supplemental support of the topical device against the perineal tissues.
55. The device of aspect 54, wherein the applicator device has an elongated body with a distal end configured to engage the outer surface of the topical device and a proximal end configured for applying a force to the topical device.
56. The device of aspect 55, wherein applying a force comprises pushing onto said applicator device by hands.
57. The device of aspect 55, wherein applying a force comprises fastening said applicator device onto the patient.
58. A method for reducing potential contact of a patient's anal discharge with perineal tissues and the baby during childbirth delivery, the method comprising the steps of:

providing a topical device having a support layer on a first side and an adhesive layer on a second side, said topical device configured for adhesive attachment to at least a portion of the perineal or perianal area of a human body;

positioning said topical device approximate above the anus; and

adhesively secure said topical device to at least a portion of the perineal or perianal area.

59. The method of aspect 58, further comprising a method for limiting perineal laceration during childbirth delivery.
60. The method of aspect 58, further comprising a method for limiting perineal laceration, tissue straining, and tissue extrusion during childbirth delivery.
61. The method of aspect 58, further comprising a method for limiting episiotomy cut from advancing toward the anus during childbirth delivery.
62. The method of aspect 58, further comprising a method for reducing third and fourth degree perineal laceration during childbirth delivery.
63. The method of aspect 58, further comprising a method for reducing medial laceration of perineal tissues during childbirth delivery.
64. The method of aspect 58, further comprising a method for diverting medial laceration of perineal tissues during childbirth delivery.
65. A topical device for reducing potential contact of a patient's anal discharge with perineal tissues and the baby during childbirth delivery comprising a support layer on a first side and an adhesive layer on a second side, said topical device configured for locating above the anus and for adhesive engagement to at least a portion of the perineal or perianal area of a human body.
66. The device of aspect 65, further comprising an adhesive-free area on the patient-contacting side.
67. The adhesive-free area of aspect 66, further comprising an adhesive-free area approximately above the anus on the patient-contacting side.
68. The adhesive-free area of aspect 66, further comprising an elongated adhesive-free channel running approximately along the midline from the anus toward the coccyx on the patient-contacting side.
69. The device of aspect 65, further comprising a cap located approximately above the anus.
70. The device of aspect 69, wherein said cap is rigid, semi-rigid or flexible.
71. The device of aspect 69, wherein said cap is planar or non-planar relative to the device prior to application onto a patient.
72. The device of aspect 69, wherein said cap is either concave or convex relative to the patient-contacting side of the topical device.
73. The device of aspect 69, wherein said cap is a drainage reservoir cap with increased space between the anus and the device for temporary collection of anal discharge or secretion.
74. The drainage reservoir cap of aspect 69, further comprising a flexible pouch.
75. The drainage reservoir cap of aspect 69, further comprising an expandable membrane.
76. The drainage reservoir cap of aspect 69, further comprising a dome-shape object.
77. The device of aspect 65, further comprising drainage tubing.
78. The device of aspect 77, further comprising a drainage container connected to the drainage tubing.
79. The device of aspect 65, further comprising a drainage reservoir cap located approximately above the anus, connected drainage tubing, and a connected drainage container.
80. The device of aspect 65, wherein at least a portion of said topical device is substantially tear-resistant.
81. The device of aspect 65, wherein said topical device is substantially tear-resistant, but not cut-resistant.
82. The device of aspect 65, further comprising a release liner for protecting said adhesive layer before application to said portion of the perineal or perianal area.
83. The device of aspect 65, wherein the shape of said topical device can be one of those shown in FIGS. 15 through 31.
84. The device of aspect 65, wherein the shape of said topical device can be one of those shown in FIGS. 29 through 30.
85. The device of aspect 65, wherein at least a portion of said topical device is substantially flexible.
86. The device of aspect 65, wherein said topical device is substantially flexible.
87. The device of aspect 65, wherein at least a portion of said topical device is substantially non-stretchable.
88. The device of aspect 65, wherein said topical device is substantially non-stretchable.
89. The device of aspect 65, wherein said topical device is substantially planar.
90. The device of aspect 65, wherein said topical device is a topical adhesive patch.
91. The device of aspect 65, wherein said support layer is made of a polymeric material, a metallic material, a ceramic material, or combination thereof.
92. The device of aspect 65, wherein said polymeric material includes polyesters, polyethylene, ultra-high molecular weight polyethylene, polypropylene, polyetheretherketone, polyethersulfone, polyvinylidene fluoride, polytetrafluoroethylene, silicone, polyurethane, polyimide, polyamide, or their combinations.
93. The device of aspect 65, wherein said support layer is polymeric film, sheet or membrane of a solid or a porous configuration.
94. The device of aspect 65, wherein said support layer is a textile fabric, mesh or sheet of woven, non-woven, braided or knitted configuration.
95. The device of aspect 65, wherein said support layer is a laminate or composite of a textile layer and a polymeric layer, wherein the textile layer and the polymeric layer can be separated or intermingled.
96. The device of aspect 65, wherein at least a portion of said support layer is reinforced with a high-tear resistant material including high tenacity fibers, high modulus fibers, oriented polymeric films, high-tear resistant polymers, or combination thereof.
97. The device of aspect 65, wherein at least a portion of said adhesive layer is a hypoallergenic adhesive.
98. The device of aspect 65, wherein at least a portion of said adhesive layer is a pressure sensitive adhesive.
99. The device of aspect 65, wherein said pressure sensitive adhesive includes acrylic adhesives, modified-acrylic adhesives, silicone adhesives, rubber-based adhesives, or combinations thereof.
100. The device of aspect 65, wherein at least a portion of said adhesive layer is reinforced with a high-tear resistant material.
101. The device of aspect 65, wherein at least a portion of said adhesive layer contains an additive selected from the group comprising a solvent, a chemical agent, a pharmaceutical agent, a biological agent, or combination thereof.
102. The device of aspect 65, wherein at least a portion of said release line is made of PET films, densified kraft paper, polycoated kraft, and polyethylene films.
103. The device of aspect 65, further comprising an applicator device for supplemental support of the topical device against the perineal or perianal tissues.
104. The device of aspect 65, wherein the applicator device has an elongated body with a distal end configured to engage the outer surface of the topical device and a proximal end configured for applying a force to the topical device.
105. The device of aspect 65, wherein applying a force comprises pushing onto said applicator device by hands.
106. The device of aspect 65, wherein applying a force comprises fastening said applicator device onto the patient.
107. The device of aspect 31, wherein the shape of said topical device can be one of those shown in FIGS. 26 through 31.
108. The device of aspect 31, wherein the shape of said topical device can be one of those shown in FIGS. 27A through 27H.
109. The device of aspect 65, wherein the shape of said topical device can be one of those shown in FIGS. 31 and 32.
110. A method for supporting compromised or injured tissues in the perineal or perianal areas before, during or after childbirth delivery, the method comprising the steps of

providing a topical device having a support layer on a first side and an adhesive layer on a second side, said topical device configured for adhesive attachment to at least a portion of the perineal or perianal area of a human body;

positioning said topical device approximate at least a portion of the compromised or injured perineal or perianal area; and

adhesively secure said topical device to at least a portion of the compromised or injured perineal or perianal area.

111. The device of aspect 110, further comprising a release liner for protecting said adhesive layer before application to said portion of the perineal or perianal area.
112. The device of aspect 110, wherein the shape of said topical device can be one of those shown in FIGS. 15 through 31.
113. A method for treating compromised or injured tissues in the perineal or perianal areas before, during or after childbirth delivery, the method comprising the steps of

providing a topical device having a support layer on a first side and an adhesive layer on a second side, wherein at least a portion of said adhesive layer or said support layer contains an additive selected from the group comprising a solvent, a chemical agent, a pharmaceutical agent, a biological agent, or combination thereof, said topical device configured for adhesive attachment to at least a portion of the perineal or perianal area of a human body;

positioning said topical device approximate at least a portion of the compromised or injured perineal or perianal area; and

adhesively secure said topical device to at least a portion of the compromised or injured perineal or perianal area.

114. The device of aspect 113, further comprising a release liner for protecting said adhesive layer before application to said portion of the perineal or perianal area.
115. The device of aspect 113, wherein the shape of said topical device can be one of those shown in FIGS. 15 through 31.
116. The device of aspect 113, wherein said pharmaceutical agent can be selected from the group comprising analgesic drugs, anesthetic drugs, anti-inflammatory drugs, anti-infective drugs, anti-allergic drugs, antibiotic drugs.
117. The device of aspect 113, wherein said chemical agent can be selected from the group comprising oils, solvents, alcohols, ethanol, glycerol, dimethyl sulfoxide, elemental silver, silver compounds, particulate silver, anti-microbial metals, anti-microbial compounds, and anti-microbial solvents.
118. A method for treating sensitive or painful tissues in the perineal or perianal areas before, during or after childbirth delivery, the method comprising the steps of

providing a topical device having a support layer on a first side and an adhesive layer on a second side, wherein at least a portion of said adhesive layer or said support layer contains an additive selected from the group comprising a solvent, a chemical agent, a pharmaceutical agent, a biological agent, or combination thereof, said topical device configured for adhesive attachment to at least a portion of the perineal or perianal area of a human body;

positioning said topical device approximate at least a portion of the sensitive or painful perineal or perianal area; and

adhesively secure said topical device to at least a portion of the sensitive or painful perineal or perianal area.

119. The device of aspect 118, further comprising a release liner for protecting said adhesive layer before application to said portion of the perineal or perianal area.
120. The device of aspect 118, wherein the shape of said topical device can be one of those shown in FIGS. 15 through 31.
121. The device of aspect 118, wherein said pharmaceutical agent can be selected from the group comprising analgesic drugs, anesthetic drugs, anti-inflammatory drugs, anti-infective drugs, anti-allergic drugs, and antibiotic drugs.
122. The device of aspect 121, wherein said analgesic drugs can be selected from the group comprising topical analgesics, opioid analgesics, narcotic analgesics, rubefacients, nonsteroidal anti-inflammatory drugs, local anesthetics, lidocane, benzocaine benzydamine, mucopolysaccharide polysulphate, and salicylamide.
123. The device of aspect 31, wherein at least a portion of said support layer is reinforced or laminated with at least one reinforcement member comprising deformable fiber, filament, yarn, thread, wire, cable, tether, strip, or bar that is made of polymeric, ceramic, metallic materials, or their composites.
124. The device of aspect 123, wherein said reinforcement member also functions as a support frame or support element.
125. The device of aspect 31, wherein at least a portion of said support layer is reinforced or laminated with at least one reinforcement layer comprising tape, strip, film, sheet, knitted fabric, woven fabric, non-woven mesh, braided construct, or combinations thereof.
126. The device of aspect 125, wherein said reinforcement layer functions as a support frame or element.
127. The device of aspect 123 or 125, wherein at least a portion of said device is further supported with at least one support frame or support element.
128. The device of aspect 127, wherein said support frame or support element is less flexible than one of said support layer, said reinforcement members, or said reinforcement layers.
129. The device of aspect 31, wherein at least a portion of said support layer is supported with at least one support frame or support element.
130. The device of aspect 129, wherein said support frame or support element is less flexible than said support layer.

What is claimed is:

1. A flexible topical device comprising a support layer and an adhesive layer disposed on a surface of the support layer, wherein the support layer comprises a tear-resistant material, the topical device is deformable to follow a contour of a surface of a tissue on which the device is to be attached, and the adhesive layer comprises a biocompatible adhesive.

2. The topical device of claim 1, wherein the support layer comprises a plurality of reinforcement members embedded in a matrix.

3. The topical device of claim 1, further comprising a tear-resistant reinforcement layer that is disposed adjacent to, and overlaps at least a portion of, the support layer.

4. The topical device of claim 1, wherein the support layer is capable of preventing or limiting an initiation or propagation of a stress- or strain-induced tear in a tissue resulting from an incision made therein when the topical device is disposed on the tissue adjacent to the incision.

5. The topical device of claim 4, wherein the support layer or the adhesive layer further comprises a medicament selected from the group consisting of analgesic drugs, anesthetic drugs, anti-infective drugs, anti-inflammatory drugs, anti-allergic drugs, antibiotic drugs, and combinations thereof.

6. The topical device of claim 4, wherein the topical device comprises an opening.

7. The topical device of claim 2, wherein the support layer is capable of preventing or limiting a propagation of a tear in a tissue resulting from an incision made therein when the topical device is disposed on the tissue adjacent the incision.

8. The topical device of claim 7, wherein the support layer or the adhesive layer further comprises a medicament selected from the group consisting of analgesic drugs, anesthetic drugs, anti-infective drugs, anti-inflammatory drugs, anti-allergic drugs, antibiotic drugs, and combinations thereof.

9. The topical device of claim 3, wherein the device is configured to cover an area between the vagina and the anus and areas on both sides of the anus, but does not cover a posterior area opposite to the vagina.

10. The topical device of claim 3, wherein a first portion of the adhesive layer opposite to, and less than an area of, the reinforcement layer, and a second portion of the adhesive layer, which is contiguous with the first portion and extends away from the reinforcement layer, are free of adhesive.

11. The topical device of claim 3, wherein the device is configured to cover an area between the vagina and the anus and an area surrounding the anus, and has an opening over the anus.

12. The topical device of claim 3, wherein the support layer and the reinforcement layer are substantially non-stretchable.

13. The topical device of claim 1, wherein the support layer is substantially non-stretchable.

14. A method for preventing or limiting an initiation or propagation of a tear in a tissue that results from an incision made in the tissue, the method comprising:
attaching a flexible topical device on a surface of the tissue adjacent to, and in a direction of, the incision,
wherein the topical device comprises a tear-resistant support layer and an adhesive layer disposed on a surface of the support layer, wherein the support layer comprises a tear-resistant material, the topical device is deformable to follow a contour of a surface of the tissue on which the device is attached, and the adhesive layer comprises a biocompatible adhesive, and wherein the adhesive layer comes into contact with the surface of the tissue.

15. The method of claim 14, further comprising preparing the surface of the tissue before attaching the topical device.

16. The method of claim 14, wherein the incision is made in an episiotomy procedure and the topical device is attached to an area between the vagina and the anus.

17. The method of claim 14, wherein the topical device further comprises a tear-resistant reinforcement layer disposed adjacent to the support layer.

18. The method of claim 17, wherein the topical device has an opening over the anus.

19. The method of claim 17, wherein the topical device covers an area between the vagina and the anus and areas on both sides of the anus, but does not cover a posterior area opposite to the vagina.

20. The method of claim 17, wherein an area of the adhesive layer over the anus is free of adhesive.
